(19) United States
(12) Patent Application Publication
(10) Pub. No.: US 2009/0111778 A1
(43) Pub. Date: Apr. 30, 2009

(54) 2-Keto-Oxazoles as Modulators of Fatty Acid Amide Hydrolase

(76) Inventors: Richard Apodaca, San Diego, CA (US); J. Guy Breitenbucher, Escondido, CA (US); Matthew T. Epperson, San Diego, CA (US); Amy K. Fried, San Diego, CA (US); Daniel J. Pippel, Del Mar, CA (US); Mark Seierstad, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/094,139

(22) PCT Filed: Nov. 17, 2006
(86) PCT No.: PCT/US2006/044709

§ 371 Date: May 16, 2008

Related U.S. Application Data
(60) Provisional application No. 60/738,248, filed on Nov. 18, 2005.

(51) Int. Cl. A61K 31/60; A61K 31/195; A61K 31/167; A61K 31/192; A61K 31/135; C07D 413/14; A61K 31/4545; C07D 413/06; A61K 31/454; A61K 31/498; A61K 31/5377; A61P 29/00; A61P 25/00; A61P 3/00; A61P 15/00; A61P 27/00; A61P 37/00; A61P 9/00

(52) U.S. Cl. 514/165; 514/561; 514/629; 514/570; 514/569; 514/646; 546/194; 514/318; 546/209; 514/326; 544/353; 514/249; 544/130; 514/236.8

(57) ABSTRACT

Certain 2-keto-oxazole compounds are described, which are useful as FAAH inhibitors. Such compounds may be used in pharmaceutical compositions and methods for the treatment of disease states, disorders, and conditions mediated by fatty acid amide hydrolase (FAAH) activity. Thus, the compounds may be administered to treat, e.g., anxiety, pain, inflammation, sleep disorders, eating disorders, or movement disorders (such as multiple sclerosis).

### CROSS-REFERENCE TO RELATED APPLICATION

This application claims priority to US. Provisional Application No. 60/738,248, filed Nov. 18, 2005.

### FIELD OF THE INVENTION

The present invention relates to certain 2-keto-oxazole compounds, pharmaceutical compositions containing them, and methods of using them for the treatment of disease states, disorders, and conditions mediated by fatty acid amide hydrolase (FAAH) activity.

### BACKGROUND OF THE INVENTION

Medicinal benefits have been attributed to the cannabis plant for centuries. The primary bioactive constituent of cannabis is Δ⁹-tetrahydro-cannabinol (THC). The discovery of THC eventually led to the identification of two endogenous cannabinoid receptors responsible for its pharmacological actions, namely CB₁ and CB₂ (Goya, Exp. Opin. Ther. Patents 2000, 10, 1529). These discoveries not only established the site of action of THC, but also inspired inquiries into the endogenous agonists of these receptors, or “endocannabinoids”. The first endocannabinoid identified was the fatty acid amide anandamide (AEA). AEA itself elicits many of the pharmacological effects of exogenous cannabinoids (Piomelli, Nat. Rev. Neurosci. 2003, 4 (11), 873).

The catabolism of AEA is primarily attributable to the integral membrane bound protein fatty acid amide hydrolase (FAAH), which hydrolyzes AEA to arachidonic acid. FAAH was characterized in 1996 by Cravatt and co-workers (Cravatt, Nature 1996, 384, 83). It was subsequently determined that FAAH is additionally responsible for the catabolism of a large number of important lipid signaling fatty acid amides including: another major endocannabinoid, 2-arachidonoylglycerol (2-AG) (Science 1992, 258, 1946-1949); the sleep-inducing substance, oleamide (OEA) (Science 1995, 268, 1506); the appetite-suppressing agent, N-oleoylethanolamine (Rodriguez de Fonesca, Nature 2001, 414, 209); and the anti-inflammatory agent, palmitoylethanolamide (PEA) (Lambert, Curr. Med. Chem. 2002, 9 (6), 663).

Small-molecule inhibitors of FAAH should elevate the concentrations of these endogenous signaling lipids and thereby produce their associated beneficial pharmacological effects. There have been some reports of the effects of various FAAH inhibitors in pre-clinical models.

In particular, two carbamate-based inhibitors of FAAH were reported to have analgesic properties in animal models. In rats, BMS-1 (see WO 02/087569), which has the structure shown below, was reported to have an analgesic effect in the Chung spinal nerve ligation model of neuropathic pain, and the Hargraves test of acute thermal nociception. URB-597 was reported to have efficacy in the zero plus maze model of anxiety in rats, as well as analgesic efficacy in the rat hot plate and formalin tests (Kathuria, Nat. Med. 2003, 9 (1), 76). The sulfonylfluoride AM374 was also shown to significantly reduce spasticity in chronic relapsing experimental autoimmune encephalomyelitis (CREAE) mice, an animal model of multiple sclerosis (Baker, FASEB J. 2001, 15 (2), 300).

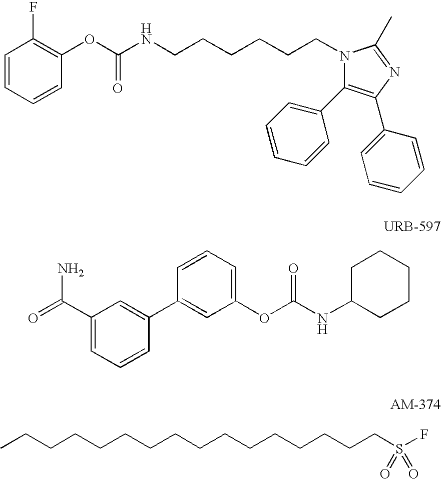

In addition, the oxazolopyridine ketone OL-135 is reported to be a potent inhibitor of FAAH, and has been reported to have analgesic activity in both the hot plate and tail emersion tests of thermal nociception in rats (WO 04/033652).

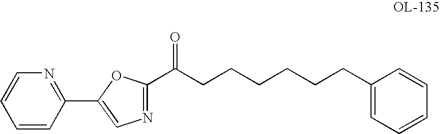

Results of research on the effects of certain exogenous cannabinoids has elucidated that a FAAH inhibitor may be useful for treating various conditions, diseases, disorders, or symptoms. These include pain, nausea/emesis, anorexia, spasticity, movement disorders; epilepsy and glaucoma. To date, approved therapeutic uses for cannabinoids include the relief of chemotherapy-induced nausea and emesis among patients with cancer and appetite enhancement in patients with HIV/AIDs who experience anorexia as a result of wasting syndrome. Two products are commercially available in some countries for these indications, namely, dronabinol (Marinol®) and nabilone.

Apart from the approved indications, a therapeutic field that has received much attention for cannabinoid use is analgesia, i.e., the treatment of pain. Five small randomized controlled trials showed that THC is superior to placebo, producing dose-related analgesia (Robson, Br. J. Psychiatry 2001, 178, 107-115). Atlantic Pharmaceuticals is reported to be developing a synthetic cannabinoid, CT-3, a 1,1-dimethyl heptyl derivative of the carboxylic metabolite of tetrahydrocannabinol, as an orally active analgesic and anti-inflammatory agent. A pilot phase II trial in chronic neuropathic pain with CT-3 was reported to have been initiated in Germany in May 2002.

A number of individuals with multiple sclerosis have claimed a benefit from cannabis for both disease-related pain and spasticity, with support from small controlled trials (Svendsen, Br. Med. J. 2004, 329, 253). Likewise, various victims of spinal cord injuries, such as paraplegia, have reported that their painful spasms are alleviated after smoking marijuana. A report showing that cannabinoids appear to control spasticity and tremor in the CREAE model of multiple sclerosis demonstrated that these effects are mediated by CB₁ and CB₂ receptors (Baker, Nature 2000, 404, 84-87). Phase 3 clinical trials have been undertaken in multiple sclerosis and spinal cord injury patients with a narrow ratio mixture of tetrahydrocannabinol/cannabidiol (THC/CBD).

Reports of small-scale controlled trials have been conducted to investigate other potential commercial uses of cannabinoids have been made. Trials in volunteers have been reported to have confirmed that oral, injected and smoked cannabinboids produced dose-related reductions in intraocular pressure (IOP) and therefore may relieve glaucoma symptoms. Ophthalmologists have prescribed cannabis for patients with glaucoma in whom other drugs have failed to adequately control intraocular pressure (Robson, 2001).

Inhibition of FAAH using a small-molecule inhibitor may be advantageous compared to treatment with a direct-acting CB₁ agonist. Administration of exogenous CB₁ agonists may produce a range of responses, including reduced nociception, catalepsy, hypothermia, and increased feeding behavior. These four in particular are termed the “cannabinoid tetrad.” Experiments with FAAH −/− mice show reduced responses in tests of nociception, but did not show catalepsy, hypothermia, or increased feeding behavior (Cravatt, Proc. Natl. Acad. Sci. USA 2001, 98 (16), 9371). Fasting caused levels of AEA to increase in rat limbic forebrain, but not in other brain areas, providing evidence that stimulation of AEA biosynthesis may be anatomically regionalized to targeted CNS pathways (Kirkham, Br. J. Pharmacol. 2002, 136, 550). The finding that AEA increases are localized within the brain, rather than systemic, suggests that FAAH inhibition with a small molecule could enhance the actions of AEA and other fatty acid amides in tissue regions where synthesis and release of these signaling molecules is occurring in a given pathophysiological condition (Piomelli, 2003).

In addition to the effects of a FAAH inhibitor on AEA and other endocannabinoids, inhibitors of FAAH's catabolism of other lipid mediators may be used in treating other therapeutic indications. For example, PEA has demonstrated biological effects in animal models of inflammation, immunosuppression, analgesia, and neuroprotection (Ueda, J. Biol. Chem. 2001, 276 (38), 35552). Oleamide, another substrate of FAAH, induces sleep (Boger, Proc. Natl. Acad. Sci. USA 2000, 97 (10), 5044; Mendelson, Neuropsychopharmacology 2001, 25, S36).

Thus, there is evidence that small-molecule FAAH inhibitors may be useful in treating pain of various etiologies, anxiety, multiple sclerosis and other movement disorders, nausea/emesis, eating disorders, epilepsy, glaucoma, inflammation, immunosuppression, neuroprotection, and sleep disorders, and potentially with fewer side effects than treatment with an exogenous cannabinoid. Various small-molecule FAAH modulators have been reported, e.g., in WO 04/033652, U.S. Pat. No. 6,462,054, U.S. Pat. No. 6,096,784, WO 99/26584, WO 97/49667, WO 96109817, and U.S. Provisional Application No. 60/640,869 (Dec. 30, 2004). However, there is still a desire for other potent FAAH modulators with desirable pharmaceutical properties.

### SUMMARY OF THE INVENTION

Certain 2-keto-oxazole derivatives have now been found to have FAAH-modulating activity.

In particular, in one general aspect the invention relates to compounds of the following Formula (I):

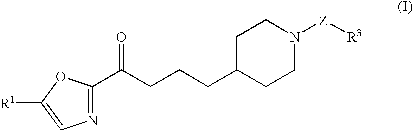

wherein:
R¹ is —H; a —CO₂C₁₋₄alkyl or —CO₂H group; or a phenyl, pyridyl, pyridazinyl, pyrimidinyl, pyrazinyl, thiophenyl, furanyl, or oxazolyl group, each unsubstituted or substituted with —CO₂H or —CO₂C₁₋₄alkyl;
Z is —C(O)—, —CO₂—, —SO₂—, —C(O)NH—, —CH₂—, or —CH(CH₃); and
R³ is:
   (a) —(CH₂)ₙ—R⁴, where n is 0, 1, or 2, and R⁴ is:
      (i) phenyl, unsubstituted or substituted with one, two, or three R^{a} moieties or where two adjacent R^{a} moieties together form —O(CH₂)₁₋₂O— or —O(CF₂)O—;
         where each R^{a} moiety is —C₁₋₇alkyl, —C₃₋₇cycloalkyl, —C₂₋₇alkenyl, —OH, —OC₁₋₇alkyl, —OC₃₋₇cycloalkyl, phenyl unsubstituted or substituted with R^{b}, phenoxy unsubstituted or substituted with R^{b}, furanyl, thiophenyl, imidazolyl, fluoro, chloro, bromo, iodo, —CF₃, —OCF₃, —SC₁₋₄alkyl, —SO₂C₁₋₄alkyl, —SOC₁₋₄alkyl, —CN, —CO₂C₁₋₄alkyl, —CO₂H, —COC₁₋₄alkyl, —SO₂NR^{c}R^{d}, —NR^{c}SO₂R^{d}, —C(O)NR^{c}R^{d}, —NR^{c}C(O)R^{d}, or —N(R^{c})R^{d};
            where R^{b} is selected from the group consisting of —C₁₋₄alkyl, —OC₁₋₄alkyl, fluoro, chloro, bromo, iodo, —CN, —OH, —CF₃, —OCF₃, and —NO₂; and
            where R^{c} and R^{d} are each independently —H or —C₁₋₇alkyl, or R^{c} and R^{d} taken together form a 3- to 7-membered heterocycloalkyl ring;
      (ii) a five- or six-membered monocyclic heteroaryl ring, unsubstituted or substituted with one or two R^{a} moieties as defined above;
      (iii) naphthyl, unsubstituted or substituted one or two R^{e} moieties, where each R^{e} moiety is independently selected from the group consisting of —C₁₋₄alkyl, —OC₁₋₄alkyl, fluoro, chloro, bromo, iodo, —CN, —OH, —CF₃, —OCF₃, and —NO₂;
      (iv) a nine- or ten-membered fused bicyclic heteroaryl, unsubstituted or substituted with one or two R^{e} moieties; or
      (v) —C₃₋₉cycloalkyl;
   (b) —(CH₂)ₓO(CH₂)_{y}R⁴, where when Z is —C(O)—, —SO₂—, or —CH₂—, x is 1 or 2 and y is 0, 1, or 2, and when Z is —CO₂— or —C(O)NH—, x is 2 and y is 0, 1, or 2;
   (c) —C₂₋₉alkyl; or
   (d) —C₂₋₉alkyl, where one carbon chain member (in formula (I)) is replaced by nitrogen or oxygen;
      or a pharmaceutically acceptable salt, pharmaceutically acceptable prodrug, or pharmaceutically active metabolite of such compound.

In another general aspect, the invention relates to compounds of the following Formula (IA):

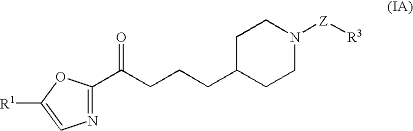

wherein:
R¹ is —H, or a phenyl, pyridyl, pyridazinyl, pyrimidinyl, pyrazinyl, thiophenyl, furanyl, or oxazolyl group;
Z is —C(O)—, —CO₂—, —SO₂—, —C(O)NH—, or —CH₂—; and
R³ is:
   (a) —(CH₂)ₙ—R⁴, where n is 0, 1, or 2, and R⁴ is:
      (i) phenyl, unsubstituted or substituted with one, or two, or three R^{a} moieties or where two adjacent R^{a} moieties together form —O(CH₂)₁₋₂O— or —O(CF₂)O—;
         where each R^{a} moiety is —C₁₋₇alkyl, —C₃₋₇cycloalkyl, —C₂₋₇alkenyl, —OH, —OC₁₋₇alkyl, phenyl unsubstituted or substituted with R^{b}, phenoxy unsubstituted or substituted with R^{b}, furanyl, thiophenyl, fluoro, chloro, bromo, iodo, —CF₃, —OCF₃, —SC₁₋₄alkyl, —SO₂C₁₋₄alkyl, —SOC₁₋₄alkyl, —CN, —CO₂C₁₋₄alkyl, —CO₂H, —COC₁₋₄alkyl, —SO₂NR^{c}R^{d}, —NR^{c}SO₂R^{d}, —C(O)NR^{c}R^{d}, —NR^{c}C(O)R^{d}, or N(R^{c})R^{d};
         where R^{b} is selected from the group consisting of —C₁₋₄alkyl, —OC₁₋₄alkyl, fluoro, chloro, bromo, iodo, —CN, —OH, —CF₃, —OCF₃, and —NO₂; and
         where R^{c} and R^{d} are each independently —H or —C₁₋₇alkyl, or R^{c} and R^{d} taken together form a 3- to 7-membered heterocycloalkyl ring;
      (ii) a five- or six-membered monocyclic heteroaryl ring, unsubstituted or substituted with one or two R^{a} moieties as defined above;
      (iii) naphthyl, unsubstituted or substituted one or two R^{e} moieties, where each R^{e} moiety is independently selected from the group consisting of —C₁₋₄alkyl, —OC₁₋₄alkyl, fluoro, chloro, bromo, iodo, —CN, —OH, —CF₃, —OCF₃, and —NO₂;
      (iv) a nine- or ten-membered fused bicyclic heteroaryl, unsubstituted or substituted with one or two R^{e} moieties; or
      (v) —C₃₋₉cycloalkyl;
   (b) —(CH₂)ₓO(CH₂)_{y}R⁴ where when Z is —C(O)—, —SO₂—, or —CH₂—, x is 1 or 2 and y is 0, 1, or 2, and when Z is —CO₂— or —C(O)NH—, x is 2 and y is 0, 1, or 2;
   (c) —C₂₋₉alkyl; or
   (d) —C₂₋₉alkyl, where one carbon chain member is replaced by nitrogen or oxygen;
      or a pharmaceutically acceptable salt, pharmaceutically acceptable prodrug, or pharmaceutically active metabolite of such compound.

In preferred embodiments, the compound of Formula (I) or (IA) is a compound specifically described or exemplified in the detailed description below.

In a further general aspect, the invention relates to pharmaceutical compositions each comprising: (a) an effective amount of an agent selected from compounds of Formula (I) or (IA) and pharmaceutically acceptable salts, pharmaceutically acceptable prodrugs, and pharmaceutically active metabolites thereof; and (b) a pharmaceutically acceptable excipient.

In another general aspect, the invention is directed to a method of treating a subject suffering from or diagnosed with a disease, disorder, or medical condition mediated by FAAH activity, comprising administering to the subject in need of such treatment an effective amount of a compound of Formula (I) or (IA), or a pharmaceutically acceptable salt, pharmaceutically acceptable prodrug, or pharmaceutically active metabolite of such compound.

In certain preferred embodiments of the inventive method, the disease, disorder, or medical condition is selected from: anxiety, pain, sleep disorders, eating disorders, inflammation, multiple sclerosis and other movement disorders (e.g., convulsions), HIV wasting syndrome, closed head injury, stroke, Alzheimer's disease, epilepsy, Tourette's syndrome, Niemann-Pick disease, Parkinson's disease, Huntington's chorea, optic neuritis, autoimmune uveitis, symptoms of drug withdrawal, nausea, emesis, sexual dysfunction, post-traumatic stress disorder, cerebral vasospasm, glaucoma, irritable bowel syndrome, inflammatory bowel disease, immunosuppression, gastroesophageal reflux disease, paralytic ileus, secretory diarrhea, gastric ulcer, rheumatoid arthritis, unwanted pregnancy, hypertension, cancer, hepatitis, allergic airway disease, auto-immune diabetes, intractable pruritis, and neuroinflammation.

Additional embodiments, features, and advantages of the invention will be apparent from the appended claims, which are incorporated into this summary by reference, as well as from the following detailed description.

### DETAILED DESCRIPTION OF INVENTION AND ITS PREFERRED EMBODIMENTS

The invention may be more fully appreciated by reference to the following description, including the following glossary of terms and the concluding examples. For the sake of brevity, the disclosures of the publications cited in this specification are herein incorporated by reference.

As used herein, the terms “including”, “containing” and “comprising” are used herein in their open, non-limiting sense.

The term “alkyl” refers to a straight- or branched-chain alkyl group having from 1 to 12 carbon atoms in the chain. Exemplary alkyl groups include methyl (Me, which also may be structurally depicted by /), ethyl (Et), n-propyl, isopropyl, butyl, isobutyl, sec-butyl, tert-butyl (tBu), pentyl, isopentyl, tert-pentyl, hexyl, isohexyl, and the like.

The term “alkenyl” refers to a straight- or branched-chain alkenyl group having from 2 to 12 carbon atoms in the chain. (The double bond of the alkenyl group is formed by two sp² hybridized carbon atoms). Illustrative alkenyl groups include prop-2-enyl, but-2-enyl, but-3-enyl, 2-methylprop-2-enyl, hex-2-enyl, and the like.

The term “heteroaryl” refers to a monocyclic, fused bicyclic, or fused polycyclic aromatic heterocycle (ring structure having ring atoms selected from carbon atoms as well as nitrogen, oxygen, and sulfur heteroatoms) having from 3 to 12 ring atoms per heterocycle. Illustrative examples of heteroaryl groups include the following moieties:

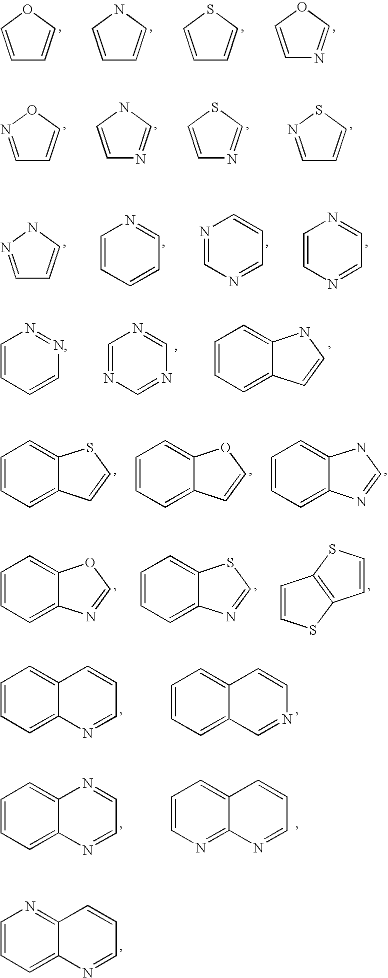

and the like.

The term “cycloalkyl” refers to a saturated or partially saturated, monocyclic, fused polycyclic, or spiro polycyclic, carbocycle having from 3 to 12 ring atoms per carbocycle. Illustrative examples of cycloalkyl groups include the following moieties:

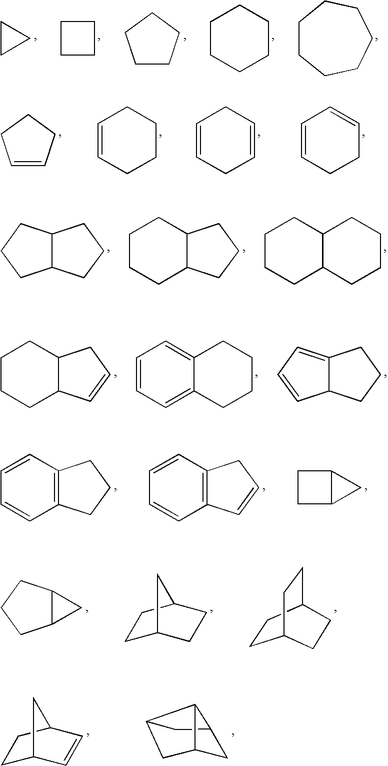

and the like.

A “heterocycloalkyl” refers to a monocyclic, fused polycyclic, or spiro polycyclic, ring structure that is saturated or partially saturated and has from 3 to 12 ring atoms per ring structure selected from C atoms and N, O, and S heteroatoms. Illustrative examples of heterocycloalkyl groups include:

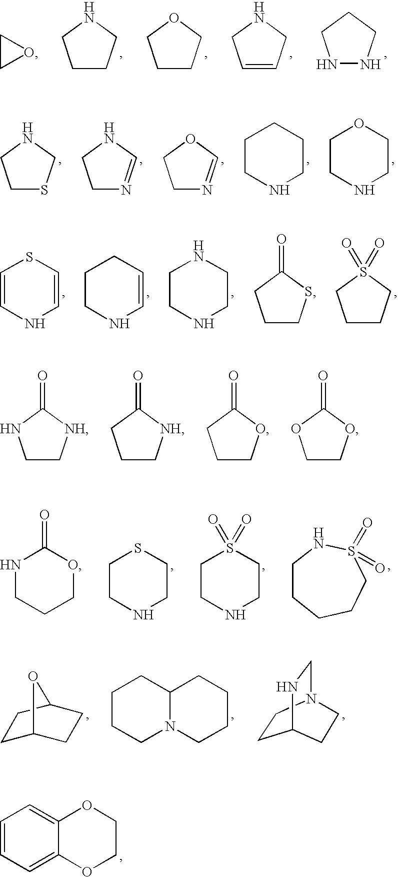

and the like.

The term “halogen” represents chlorine, fluorine, bromine or iodine. The term “halo” represents chloro, fluoro, bromo or iodo.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system.

When referring to any formula given herein, the selection of a particular moiety from a list of possible species for a specified variable is not intended to define the moiety for the variable appearing elsewhere. In other words, where a variable appears more than once, the choice of the species from a specified list is independent of the choice of the species for the same variable elsewhere in the formula.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof.

Furthermore, certain structures may exist as geometric isomers (i.e., cis and trans isomers), as tautomers, or as atropisomers. Additionally, any formula given herein is intended to represent hydrates, solvates, and polymorphs of such compounds, and mixtures thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, ¹²⁵I, respectively. Various isotopically labeled compounds of the present invention, for example those into which radioactive isotopes such as ³H, ¹¹C, and ¹⁴C are incorporated. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the moiety for the variable appearing elsewhere. In other words, where a variable appears more than once, the choice of the species from a specified list is independent of the choice of the species for the same variable elsewhere in the formula.

In preferred embodiments of Formula (I), R¹ is —H. In further preferred embodiments, R¹ is a —CO₂CH₃ or —CO₂H group. In still further preferred embodiments, R¹ is a pyridyl group, unsubstituted or substituted with —CO₂H.

In preferred embodiments, R³ is R⁴ and R⁴ is tert-butyl, phenyl or pyridyl.

In preferred embodiments, each R^{a} moiety is methyl, isopropyl, tert-butyl, isopropoxy, cyclohexyloxy, phenyl, phenoxy, 1H-imidazol-1-yl, fluoro, chloro, or CF₃.

In preferred embodiments of Formula (I) or (IA), R¹ is —H, 2-pyridyl, or 2-furanyl.

Preferably, Z is —C(O)—, —CO₂—, —SO₂—, or —CH₂—. More preferably, Z is —C(O)— or —CH₂—

In preferred embodiments, R³ is —R⁴, —CH₂—R⁴, —(CH₂)₂—R⁴, —CH₂—O—R⁴, —CH₂—O—CH₂—R⁴, —CH₂—O—R⁴, —CH₂—O—CH₂—R⁴, —CH₂—O—CH₂CH₂—R⁴, —CH₂CH₂—O—R⁴, —CH₂CH₂—O—CH₂—R⁴, or —CH₂CH₂—O—CH₂CH₂—R⁴, wherein R⁴ is phenyl, pyridyl, pyridazinyl, pyrimidinyl, pyrazinyl, pyrrolyl, thiophenyl, furanyl, pyrazolyl, imidazolyl, oxazolyl, isoxazolyl, thiazolyl, oxadiazolyl, thiadiazolyl, naphthyl, cyclopropyl, cyclobutyl, cyclopentyl, cyclohexyl, cycloheptyl, cyclooctyl, or cyclononyl, each optionally substituted as described above. Alternatively, R³ is ethyl, propyl, isopropyl, 2-methylpropyl, 2,2-dimethylpropyl, 1,2-dimethylpropyl, butyl, isobutyl, sec-butyl, tert-butyl, 2-methylbutyl, pentyl, isopentyl, hexyl, octyl, hydroxyethyl, hydroxypropyl, methoxyethyl, ethoxyethyl, methoxypropyl, methoxybutyl, aminoethyl, 2-methylaminoethyl, or 2-methylaminopropyl. More preferably, R³ is —R⁴, —CH₂—R⁴, —(CH₂)₂—R⁴, —CH₂—O—R⁴, —CH₂—O—CH₂—R⁴, —CH₂—O—R⁴, —CH₂—O—CH₂—R⁴, —CH₂—O—CH₂CH₂—R⁴, —CH₂CH₂—O—R⁴, —CH₂CH₂—O—CH₂—R⁴, or —CH₂CH₂—O—CH₂CH₂—R⁴, wherein R⁴ is phenyl, pyridyl, isoxazolyl, furanyl, naphthyl, quinolinyl, quinoxalinyl, naphthyridinyl, cyclopentyl, or cyclohexyl, each optionally substituted as described above. Alternatively, R³ is ethyl, propyl, isopropyl, 2,2-dimethylpropyl, butyl, isobutyl, sec-butyl, tert-butyl, hexyl, octyl, 3-methylbutyl, methoxyethyl, or ethoxyethyl. Exemplary R³ moieties include phenyl, 3-phenoxyphenyl, 4-phenoxyphenyl, 4-fluorophenyl, 3-fluorophenyl, 4-chlorophenyl, 3-chlorophenyl, benzo[1,3]dioxolyl, 4-methylphenyl, 3-methylphenyl, 4-t-butoxyphenyl, 2-methylphenyl, 2,3-difluorophenyl, 4-isobutylphenyl, 4-t-butylphenyl, 3,4-dibromophenyl, 3,4-dichlorophenyl, 3-chloro-4-fluorophenyl, 4-methoxyphenyl, 3-methoxyphenyl, 4-isopropylphenyl, 4-isopropoxyphenyl, 4-ethylphenyl, 3-biphenyl, 4-biphenyl, 2-chlorophenyl, 2-bromophenyl, 2-methoxyphenyl, 4-dimethylaminophenyl, 4-diethylaminophenyl, 3-bromophenyl, 4-bromophenyl, 2,3-dimethylphenyl, 4-cyclohexylphenyl, 4-pyrrolidin-1-ylphenyl, 4-piperidin-1-ylphenyl, 4-morpholin-1-ylphenyl, 2,5-difluorophenyl, 2,4-difluorophenyl, 3,4-difluorophenyl, phenethyl, benzyl, 4-methylbenzyl, 3-methylbenzyl, 4-chlorobenzyl, 3-chlorobenzyl, 2-chlorobenzyl, 2-(4-phenoxyphenyl)ethyl, o-tolylethyl, p-tolylethyl, 2-(4-chlorophenyl)ethyl, 2,4,6-trifluorophenyl, 2,3,5-trifluorophenyl, 2,2-difluoro-benzo[1,3]dioxol-5-yl, naphthyl, pyridin-2-yl, 6-bromo-pyridin-2-yl, 6-methyl-pyridin-2-yl, pyridin-3-yl, pyridin-4-yl, 6-methoxy-pyridin-3-yl, 6-chloro-pyridin-3-yl, 5-bromo-pyridin-3-yl; 6-bromo-pyridin-3-yl, 6-bromo-pyridin-3-yl, 6-p-tolyloxy-pyridin-3-yl, 6-(3-methoxy-phenyl)-pyridin-3-yl, 6-phenoxy-pyridin-3-yl, 6-morpholin-4-yl-pyridin-3-yl, 3,4,5,6-tetrahydro-2H-[1,2′]bipyridinyl-5′-yl, 6-furan-2-yl-pyridin-3-yl, 6-thiophen-2-yl-pyridin-3-yl, 6-thiophen-3-yl-pyridin-3-yl, 6-(3-cyanophenyl)-pyridin-3-yl, pyridinylethyl, 3-quinolinyl, 2-quinolinyl, 4-quinolinyl, 2-chloro-quinolin-3-yl, 2-chloro-6-methyl-quinolin-3-yl, 2-chloro-8-methyl-quinolin-3-yl, 2-chloro-6-methoxy-quinolin-3-yl, [1,8]naphthyridin-2-yl, quinoxalin-2-yl, 3-methyl-isoxazol-5-ylmethyl, 2-furanyl, cyclopentyl, cyclohexyl, cyclopentylmethyl, cyclohexylmethyl, cyclopentylethyl, cyclohexylethyl, phenoxymethyl, 4-chlorophenoxymethyl, benzyloxymethyl, 2-benzyloxyethyl, ethyl, propyl, isopropyl, 2,2-dimethylpropyl, butyl, isobutyl, sec-butyl, tert-butyl, hexyl, octyl, 3-methylbutyl, 2-methoxyethyl, or 2-ethoxyethyl.

The invention includes also pharmaceutically acceptable salts of the compounds represented by Formula (I) or (IA), such as of those described above. Pharmaceutically acceptable salts of the specific compounds exemplified herein are especially preferred.

A “pharmaceutically acceptable salt” is intended to mean a salt of a free acid or base of a compound represented by Formula (I) or (IA) that is not toxic, biologically intolerable, or otherwise biologically undesirable. See, generally, S. M. Berge et al., “Pharmaceutical Salts”, J. Pharm. Sci., 1977, 66:1-19, and Handbook of Pharmaceutical Salts, Propertions, Selection, and Use, Stahl and Wermuth, Eds., Wiley-VCH and VHCA, Zurich, 2002. Preferred pharmaceutically acceptable salts are those that are pharmacologically effective and suitable for contact with the tissues of patients without undue toxicity, irritation, or allergic response. A compound of Formula (I) or (IA) may possess a sufficiently acidic group, a sufficiently basic group, or both types of functional groups, and accordingly react with a number of inorganic or organic bases, and inorganic and organic acids, to form a pharmaceutically acceptable salt. Exemplary pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of Formula (I) or (IA) contains a basic nitrogen, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or the like.

If the compound of Formula (I) or (IA) is an acid, such as a carboxylic acid or sulfonic acid, the desired pharmaceutically acceptable salt may be prepared by any suitable method, for example, treatment of the free acid with an inorganic or organic base, such as an amine (primary, secondary or tertiary), an alkali metal hydroxide, or alkaline earth metal hydroxide, or the like. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, and piperazine, and inorganic salts derived from sodium, calcium, potassium, magnesium, manganese, iron, copper, zinc, aluminum, and lithium.

The invention also relates to treatment methods employing pharmaceutically acceptable prodrugs of the compounds of Formula (I) or (IA). The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I) or (IA)). A “pharmaceutically acceptable prodrug” is a prodrug that is not toxic, biologically intolerable, or otherwise biologically unsuitable for administration to the subject. Illustrative procedures for the selection and preparation of suitable prodrug derivatives are described, for example, in “Design of Prodrugs”, ed. H. Bundgaard, Elsevier, 1985.

Exemplary prodrugs include compounds having an amino acid residue, or a polypeptide chain of two or more (e.g., two, three or four) amino acid residues, covalently joined through an amide or ester bond to a free amino, hydroxy, or carboxylic acid group of a compound of Formula (I) or (IA). Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs may be produced, for instance, by derivatizing free carboxyl groups of structures of Formula (I) or (IA) as amides or alkyl esters. Exemplary amides include those derived from ammonia, primary C₁₋₆alkyl amines and secondary di(C₁₋₆alkyl) amines. Secondary amines include 5- or 6-membered heterocycloalkyl or heteroaryl ring moieties. Preferred amides are derived from ammonia, C₁₋₃alkyl primary amines, and di(C₁₋₂alkyl)amines. Exemplary esters of the invention include C₁₋₇alkyl, C₅₋₇cycloalkyl, phenyl, and phenyl(C₁₋₆alkyl) esters. Preferred esters include methyl esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Adv. Drug Delivery Rev. 1996, 19, 115. Carbamate derivatives of hydroxy and amino groups may also yield prodrugs. Carbonate derivatives, sulfonate esters, and sulfate esters of hydroxy groups may also provide prodrugs. Derivatization of hydroxy groups as (acyloxy)methyl and (acyloxy)ethyl ethers, wherein the acyl group may be an alkyl ester, optionally substituted with one or more ether, amine, or carboxylic acid functionalities, or where the acyl group is an amino acid ester as described above, is also useful to yield prodrugs. Prodrugs of this type may be prepared as described in J. Med. Chem. 1996, 39, 10. Free amines can also be derivatized as amides, sulfonamides or phosphonamides. All of these prodrug moieties may incorporate groups including ether, amine, and carboxylic acid functionalities.

Pharmaceutically active metabolites may also be used in the methods of the invention. A “pharmaceutically active metabolite” means a pharmacologically active product of metabolism in the body of a compound of Formula (I) or (IA) or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 224-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

The compounds of Formula (I) or (IA) and their pharmaceutically acceptable salts, pharmaceutically acceptable prodrugs, and pharmaceutically active metabolites (collectively, “agents”) of the present invention are useful as FAAH inhibitors in the methods of the invention. The agents may be used in the inventive methods for the treatment or prevention of medical conditions, diseases, or disorders mediated through inhibition or modulation of FAAH, such as those described herein. Agents according to the invention may therefore be used as an analgesic, neuroprotectant, sedative, appetite stimulant, or contraceptive.

Exemplary medical conditions, diseases, and disorders include anxiety, pain, sleep disorders, eating disorders, inflammation, multiple sclerosis and other movement disorders, HIV wasting syndrome, closed head injury, stroke, Alzheimer's disease, epilepsy, Tourette's syndrome, epilepsy, Niemann-Pick disease, Parkinson's disease, Huntington's chorea, optic neuritis, autoimmune uveitis, symptoms of drug withdrawal, nausea, emesis, sexual dysfunction, post-traumatic stress disorder, or cerebral vasospasm.

Thus, the pharmaceutical agents may be used to treat subjects diagnosed with or suffering from a disease, disorder, or condition mediated through FAAH activity. The term “treat” or “treating” as used herein is intended to refer to administration of an agent or composition of the invention to a subject for the purpose of effecting a therapeutic or prophylactic benefit through modulation of FAAH activity. Treating includes reversing, ameliorating, alleviating, inhibiting the progress of, lessening the severity of, or preventing a disease, disorder, or condition, or one or more symptoms of such disease, disorder or condition mediated through modulation of FAAH activity. The term “subject” refers to a mammalian patient in need of such treatment, such as a human. “Modulators” include both inhibitors and activators, where “inhibitors” refer to compounds that decrease, prevent, inactivate, desensitize or down-regulate FAAH expression or activity, and “activators” are compounds that increase, activate, facilitate, sensitize, or up-regulate FAAH expression or activity.

Accordingly, the invention relates to methods of using the pharmaceutical agents described herein to treat subjects diagnosed with or suffering from a disease, disorder, or condition mediated through FAAH activity, such as: anxiety, pain, sleep disorders, eating disorders, inflammation, or movement disorders (e.g., multiple sclerosis).

Symptoms or disease states are intended to be included within the scope of “medical conditions, disorders, or diseases.” For example, pain may be associated with various-diseases, disorders, or conditions, and may include various etiologies. Illustrative types of pain treatable with a FAAH-modulating agent according to the invention include cancer pain, postoperative pain, GI tract pain, spinal cord injury pain, visceral hyperalgesia, thalamic pain, headache (including stress headache and migraine), low back pain, neck pain, musculoskeletal pain, peripheral neuropathic pain, central neuropathic pain, neurogenerative disorder related pain, and menstrual pain. HIV wasting syndrome includes associated symptoms such as appetite loss and nausea. Parkinson's disease includes, for example, levodopa-induced dyskinesia. Treatment of multiple sclerosis may include treatment of symptoms such as spasticity, neurogenic pain, central pain, or bladder dysfunction. Symptoms of drug withdrawal may be caused by, for example, addiction to opiates or nicotine. Nausea or emesis may be due to chemotherapy, postoperative, or opioid related causes. Treatment of sexual dysfunction may include improving libido or delaying ejaculation. Treatment of cancer may include treatment of glioma. Sleep disorders include, for example, sleep apnea, insomnia, and disorders calling for treatment with an agent having a sedative or narcotic-type effect. Eating disorders include, for example, anorexia or appetite loss associated with a disease such as cancer or HIV infection/AIDS.

In a treatment method according to the invention, an effective amount of a pharmaceutical agent according to the invention is administered to a subject suffering from or diagnosed as having such a disease, disorder, or condition. An “effective amount” means an amount or dose sufficient to generally bring about the desired therapeutic or prophylactic benefit in patients in need of such treatment.

Effective amounts or doses of the agents of the present invention may be ascertained by routine methods such as modeling, dose escalation studies or clinical trials, and by taking into consideration routine factors, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician. An exemplary dose is in the range of from about 0.001 to about 200 mg of agent per kg of subject's body weight per day, preferably about 0.05 to 100 mg/kg/day, or about 1 to 35 mg/kg/day, in single or divided dosage units (e.g., BID, TID, QID). For a 70-kg human, an illustrative range for a suitable dosage amount is from about 0.05 to about 7 g/day, or about 0.2 to about 2.5 g/day.

Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

In addition, the agents of the invention may be used in combination with additional active compounds in the treatment of the above conditions. The additional compounds may be coadministered separately with an agent of Formula (I) or (IA) or included with such an agent as an additional active ingredient in a pharmaceutical composition according to the invention. In an exemplary embodiment, additional active compounds are those that are known or discovered to be effective in the treatment of conditions, disorders, or diseases mediated by FAAH activity, such as another FAAH modulator or a compound active against another target associated with the particular condition, disorder, or disease. The combination may serve to increase efficacy (e.g., by including in the combination a compound potentiating the potency or effectiveness of an agent according to the invention), decrease one or more side effects, or decrease the required dose of the agent according to the invention. In one illustrative embodiment, a composition according to the invention may contain one or more additional active ingredients selected from opioids, NSAIDs (e.g., ibuprofen, cyclooxygenase-2 (COX-2) inhibitors, and naproxen), gabapentin, pregabalin, tramadol, acetaminophen, and aspirin.

The agents of the invention are used, alone or in combination with one or more other active ingredients, to formulate pharmaceutical compositions of the invention. A pharmaceutical composition of the invention comprises: (a) an effective amount of a pharmaceutical agent in accordance with the invention; and (b) a pharmaceutically acceptable excipient.

A “pharmaceutically acceptable excipient” refers to a substance that is not toxic, biologically intolerable, or otherwise biologically unsuitable for administration to a subject, such as an inert substance, added to a pharmacological composition or otherwise used as a vehicle, carrier, or diluent to facilitate administration of a pharmaceutical agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the pharmaceutical agents may be prepared using suitable pharmaceutical excipients and compounding techniques now or later known or available to those skilled in the art. The compositions may be administered in the inventive methods by oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the invention can be provided in the form of tablets or capsules, or as a solution, emulsion, or suspension. To prepare the oral compositions, the agents may be formulated to yield a dosage of, e.g., from about 0.05 to about 50 mg/kg daily, or from about 0.05 to about 20 mg/kg daily, or from about 0.1 to about 10 mg/kg daily.

Oral tablets may include the active ingredient mixed with pharmaceutically acceptable excipients such as inert diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The agents of this invention may also be administered by non-oral routes. For example, the compositions may be formulated for rectal administration as a suppository. For parenteral use, including intravenous, intramuscular, intraperitoneal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of agent, admixed with a pharmaceutical carrier over a period ranging from several minutes to several days.

For topical administration, the agents may be mixed with a pharmaceutical carrier at a concentration of about 0.1% to about 10% of drug to vehicle. Another mode of administering the agents of the invention may utilize a patch formulation to affect transdermal delivery.

Agents may alternatively be administered in methods of this invention by inhalation, via the nasal or oral routes, e.g., in a spray formulation also containing a suitable carrier.

Exemplary agents useful in methods of the invention will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). One skilled in the art will recognize that compounds of Formula (I) include compounds of Formula (IA).

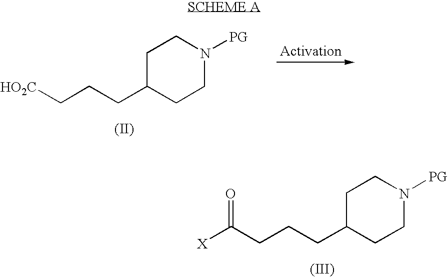

Referring to Scheme A, protected piperidine acids of formula (II), where PG is a suitable nitrogen protecting group, are commercially available or are prepared from 4-piperidin-4-yl-butyric acid. Preferably, PG is a tert-butoxycarbonyl (Boc) protecting group, and is installed by treatment of the piperidine with Boc₂O in the presence of a base such as DMAP or iPr₂NEt, in a solvent such as acetonitrile, THF, or dioxane; or with a base such as NaHCO₃, NaOH, or KOH, in water or tert-butanol or mixtures thereof; or under Schotten-Baumen conditions. Where piperidine acids are used in their amine salt forms, such as hydrochloride salts, additional base may be used.

Reagents (III), such as acid chlorides (where X is Cl) and Weinreb amides (where X is —N(OMe)Me) are useful in the formation of compounds of Formula (I). Acid chloride analogs are generated from acids (II) by reaction with thionyl chloride in the presence of pyridine. Acids (II) may be converted to Weinreb amides by 1) treatment with a suitable chloroformate reagent, such as isobutylchloroformate, in the presence of excess base such as Et₃N, in a solvent such as CH₂Cl₂ or DCE, and 2) addition of N,O-dimethylhydroxylamine. Weinreb amides are also accessible from acid chlorides (III) via treatment with N,O-dimethylhydroxylamine and a suitable base such as Et₃N, in a solvent such as CH₂Cl₂ or DCE.

Esters (III), such as ethyl esters, are made from acids (II) by treatment with an alkanol, such as ethanol, under acidic conditions. Preferred conditions include reaction with acetyl chloride and ethanol at temperatures between about room temperature and reflux temperature. In an alternative embodiment, formation of the ethyl ester may be performed prior to the installation of the nitrogen protecting group, PG, or both transformations may be accomplished in one reaction step.

Said esters (III) are converted to Weinreb amides under conditions known to one skilled in the art, such as treatment with N,O-dimethylhydroxylamine hydrochloride in the presence of a Lewis acid such as AlCl₃ in a solvent such as hexanes, THF, Et₂O, or mixtures thereof. Alternatively, esters (III) may be converted to Weinreb amides (III) by reaction with the magnesium amide of N,O-dimethylhydroxylamine, which is formed by treating N,O-dimethylhydroxylamine with an alkyl Grignard reagent; such as iPrMgCl, in a dry, inert solvent, such as THF. (See, Williams et al., Tetrahedron Lett. 1995, 36 (31), 5461-5464).

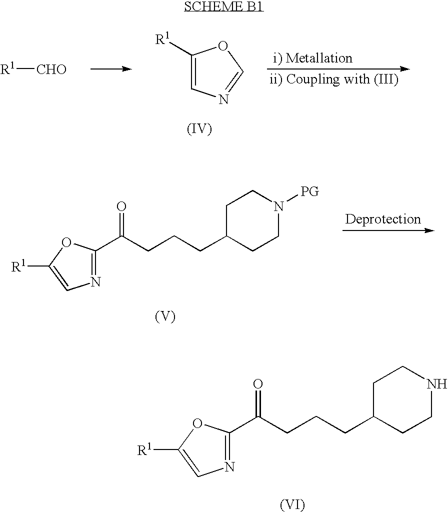

Alternatively, various inventive compounds may be prepared in accordance with Scheme B1. Referring to Scheme B1, oxazoles (IV) are commercially available or may be prepared, for example, by condensation of aldehydes R¹CHO with tosylmethyl isocyanide (TosMIC), in the presence of a suitable base such as K₂CO₃, in a solvent such as MeOH. In preparation for coupling, metallation of oxazoles (IV) may be accomplished according to various procedures. In one embodiment, oxazoles (IV) are lithiated at the 2-position by treatment with n-BuLi or sec-BuLi, at temperatures of about −78° C., in a solvent such as THF. Direct coupling of a lithiated oxazole with reagents (III) will generate ketones (V) (Boger et al., J. Med. Chem. 2005, 48 (6), 1849-1856). Alternatively, the 2-lithio-oxazoles are transmetallated in situ to their corresponding zinc reagents by treatment with ZnCl₂. Reaction solutions may be warmed to about 0° C. Subsequent in situ treatment of the zinc reagents with a copper(I) species such as CuI gives metallated oxazoles that may be coupled with compounds of formula (III), where X is Cl, to give ketones (V). (See: Boger, D. et al. PNAS 2000, 97 (10), 5044-5049).

This scheme has some features which limits its desirability for the large-scale preparation of compounds of Formula (I) and (IA). Where R¹ is a phenyl, pyridyl, pyridazinyl, pyrimidinyl, pyrazinyl, thiophenyl, furanyl, or oxazolyl group, on larger scale, the significant amounts of metal salts, as well as the viscosity of the mixture, cause inefficient stirring.

To accomplish the transformation in a scalable fashion, oxazoles (IV) are instead metallated by treatment with an alkyl Grignard reagent, such as iPrMgCl or iPrMgBr, at temperatures of about −15° C., in a solvent such as THF or Et₂O or mixtures thereof, to form the corresponding oxazole Grignard reagents. To these reagents may then be added Weinreb amides (III), where X is —N(OMe)Me, to form ketones (V). The coupling step may be performed at temperatures between −78° C. and the reflux temperature of the solvent.

This Grignard coupling protocol has several advantages. First, cryogenic temperatures below −50° C., which are problematic on large scale, are avoided. The significant amounts of copper and zinc waste generated in the Zn/Cu method are avoided by the Grignard procedure, which reduces disposal issues and cost. Finally, the Grignard method consistently gave cleaner conversions, limiting any tedious chromatography necessary to isolate the desired ketones (V).

In particular, compounds of Formula (IA) may therefore be advantageously prepared by a process comprising the steps of:

a) reacting an oxazole (IV) with iPrMgHal in an organic solvent to form an organic mixture; and

b) treating the organic mixture with 4-[3-(methoxy-methyl-carbamoyl)-propyl]-piperidine-1-carboxylic acid tert-butyl ester to form a compound of formula (V);

wherein Hal is Cl or Br, and R¹ is defined as above.

Preferably, the reacting of the oxazole (IV) with iPrMgHal is done at temperatures between about −30° C. and about 0° C. More preferably, the reacting of the oxazole (IV) with iPrMgHal is done at temperatures between about −15° C. and about 0° C.

Preferably, the treating of the organic mixture with 4-[3-(methoxy-methyl-carbamoyl)-propyl]-piperidine-1-carboxylic acid tert-butyl ester is done at temperatures between about 0° C. and the reflux temperature of the solvent. More preferably, the treating of the organic mixture with 4-[3-(methoxy-methyl-carbamoyl)-propyl]-piperidine-1-carboxylic acid tert-butyl ester is done at temperatures between about 0° C. and about 25° C.

Preferably, R¹ is —H, pyridyl, or furanyl.

Preferably, Hal is Cl.

Preferably, the reacting with iPrMgHal is reacting with two molar equivalents of iPrMgHal relative to one molar equivalent of oxazole (IV).

Preferably, the organic solvent is tetrahydrofuran (THF).

Deprotection of the PG protecting group may be accomplished using general methods known in the art. For example, where PG is a Boc group, deprotection of a compound of formula (V) may be effected by treatment with TFA, neat or in combination with CH₂Cl₂, or with HCl in Et₂O, dioxane, or EtOAc, or with a Lewis acid such as BF₃.OEt₂ in acetic acid. Thus, the method of making a compound of Formula (IA) further comprises treating a compound of formula (V) with TFA to give a compound of formula (VI).

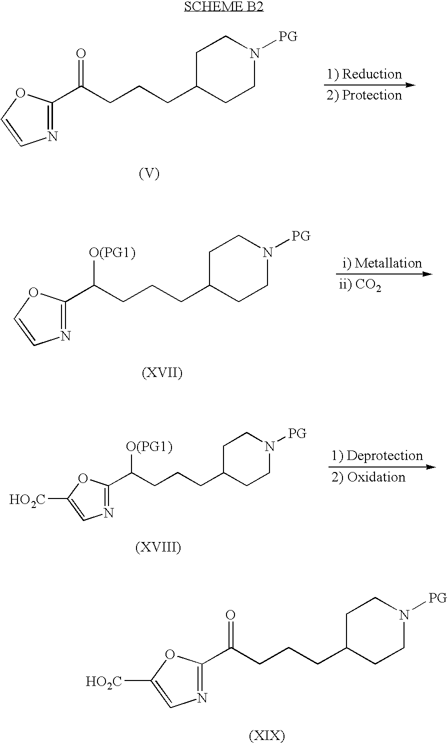

Various inventive compound of Formula (I) where R¹ is a —CO₂C₁₋₄-alkyl or —CO₂H group may be prepared according to Scheme B2. Ketones of formula (V) (which are themselves embodiments of Formula (I) or (IA)) are reduced to the corresponding secondary alcohols (XVII, where PG1 is —H) using a reagent such as NaBH₄ in a solvent such as MeOH. Installation of a suitable hydroxyl protecting group (PG1), such as a trialkylsilyl group (preferably, a tert-butyldimethylsilyl group) is accomplished using general methods known in the art to give protected alcohols (XVIII). Metallation of the oxazole group using an alkyllithium reagent, such as t-BuLi, at temperatures of about −78° C., in a solvent such as THF gives lithiated oxazoles, which are reacted in situ with CO₂ to give acids (XVIII). Deprotection of the hydroxyl group using general methods known in the art, followed by oxidation of the resulting alcohol (using, for example, Dess-Martin periodinane or a Swern oxidation) provides ketones (XIX). The carboxy group may optionally be transformed into an ester group using methods known in the art. Ketones (XIX) are converted to compounds of Formula (I) according to the methods described in the previous and subsequent Schemes.

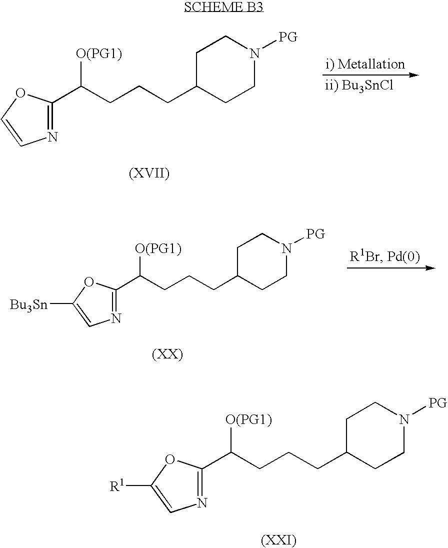

Various inventive compound of Formula (I) where R¹ is an optionally substituted phenyl, pyridyl, pyridazinyl, pyrimidinyl, pyrazinyl, thiophenyl, furanyl, or oxazolyl group may be prepared according to Scheme B3. Metallation of the oxazole group using an alkyllithium reagent, such as t-BuLi, at temperatures of about −78° C., in a solvent such as THF gives lithiated oxazoles, which are reacted in situ with Bu₃SnCl to give tin reagents (XX). Palladium-mediated coupling of the tin reagents (XX) with phenyl or heteroaryl bromides, in the presence of a suitable palladium catalyst such as Pd(PPh₃)₄, in a solvent such as dioxane, at temperatures between about room temperature and the boiling point of the solvent, yields substituted oxazoles (XXI). Oxazoles (XXI) are then converted into compounds of Formula (I) according to the methods described in the previous and subsequent Schemes. Where the R¹ substituent is further substituted with a carboxy group, one skilled in the art will recognize that reactions may be performed on the corresponding ester.

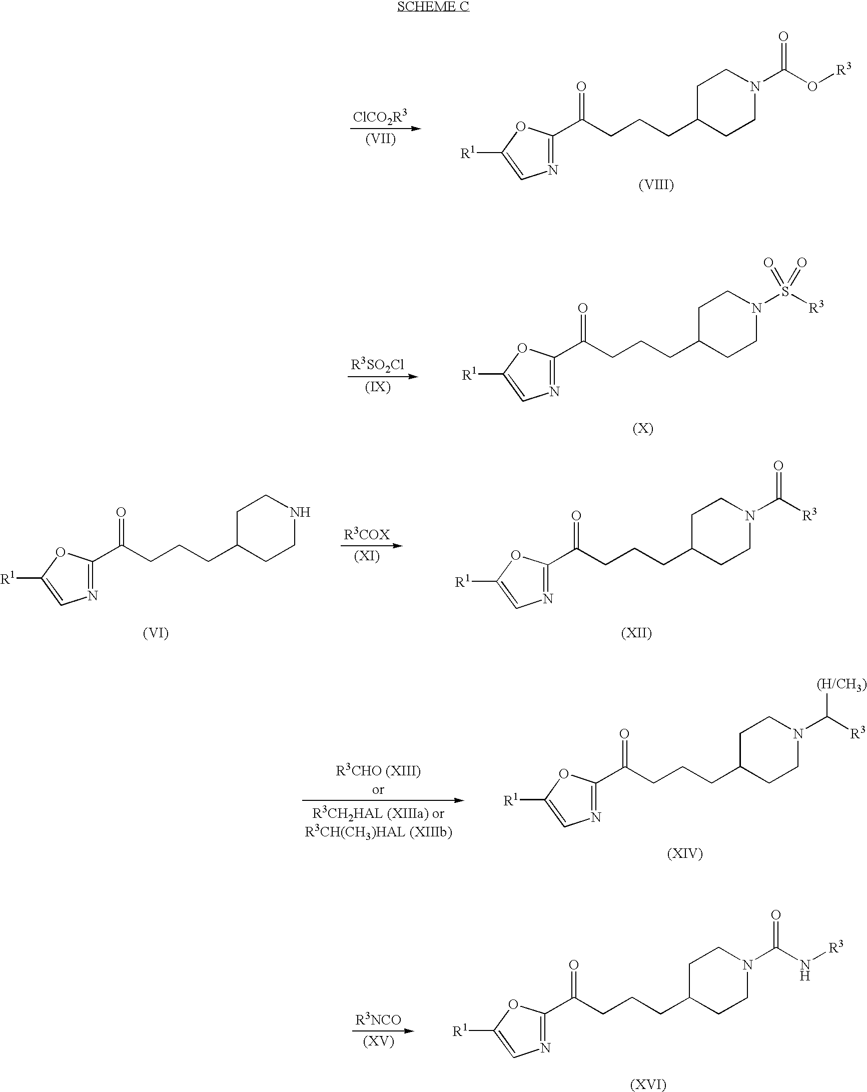

Conversion of piperidines (VI) into compounds of Formula (I) may be accomplished as shown in Scheme C. Formulae (VII), (X), (XII), (XIV), and (XVI) are within the scope of Formula (I). Carbamates (VIII) may be prepared by reaction of piperidines (VI) with a suitable chloroformate reagent (VII), in the presence of a suitable base such as Et₃N or pyridine, in a solvent such as CH₂Cl₂, DCE, or THF. Sulfonamides (X) may be prepared by reaction of piperidines (VI) with sulfonyl chlorides (IX) in the presence of a base such as Et₃N or pyridine in a solvent such as CH₂Cl₂, DCE, or THF. Amides (XII) are prepared from piperidines (VI) by reaction with acid chlorides (XI) (where X is Cl) in the presence of a base such as Et₃N or pyridine in a solvent such as CH₂Cl₂, DCE, or THF. Alternatively, piperidines (VI) may be coupled with acids (XI) (where X is OH) under peptide coupling conditions known to one skilled in the art. Amines (XIV) are generated by reaction of piperidines (IV) with an aldehyde (XIII) under reductive amination conditions known to one skilled in the art. Preferred reducing agents include NaCNBH₃ or NaB(OAc)₃H. Reactions may be performed with or without an additive such as acetic acid or ZnCl₂, in a solvent such as CH₂Cl₂, DCE, or MeOH. Preferably, reductive aminations are accomplished with NaB(OAc)₃H in DCE. Alternatively, piperidines (IV) may be alkylated using methods known in the art. For example, reaction with a suitable alkylating agent (XIIIa) or (XIIIb), where X is Cl or Br, in the presence of a base such as K₂CO₃ or Na₂CO₃, with optional additives such as KI, in a solvent such as THF or acetonitrile, will yield amines (XIV). Ureas of formula (XVI) are prepared by reacting piperidines (IV) with isbcyanates (XV) in the presence of a base such as Et₃N or pyridine, in a solvent such as CH₂Cl₂, DCE, or THF.

The following examples are provided to further illustrate the invention and various preferred embodiments.

### EXAMPLES

### Chemistry

In obtaining the characterization data described in the examples below, the following analytical protocols were followed as indicated.

NMR spectra were obtained on Brucker model DRX spectrometers. The format of the ¹H NMR data below is: chemical shift in ppm downfield of the tetramethylsilane reference (multiplicity, coupling constant J in Hz, integration).

Silica gel was used for all chromatographic purification unless otherwise noted. Where solutions were “concentrated”, they were concentrated using a rotary evaporator under reduced pressure. Unless otherwise specified, reaction solutions were stirred at room temperature (rt) under a nitrogen atmosphere.

Mass spectra were obtained on an Agilent series 1100 MSD using electrospray ionization (ESI) in either positive or negative modes as indicated. Calculated mass corresponds to the exact mass.

Thin-layer chromatography was performed using Merck silica gel 60 F₂₅₄ 2.5 cm×7.5 cm×250 μm or 5.0 cm×10.0 cm×250 μm pre-coated silica gel plates. Preparative thin-layer chromatography was performed using EM Science silica gel 60 F₂₅₄ 20 cm×20 cm×0.5 mm pre-coated plates with a 20 cm×4 cm concentrating zone.

Reversed-phase HPLC was performed on a Hewlett Packard HPLC Series 1100, with a Phenomenex Luna C18 (5 μm, 4.6×150 mm) column. Detection was done at λ=230, 254 and 280 nm. The flow rate was 1 mL/min. The gradient was 10 to 99% acetonitrile/water (0.05% trifluoroacetic acid) over 5.0 min.

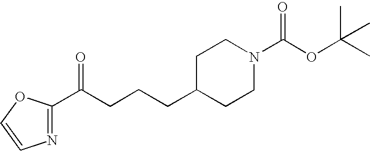

### Example 1

### 4-(4-Oxazol-2-yl-4-oxo-butyl)-piperidine-1-carboxylic acid tert-butyl ester

To a stirred solution of oxazole (400 μL) in THF (30 mL) at −78° C. was added nBuLi (1.6 M in hexanes, 4.25 mL), and the resulting pale yellow solution was stirred for 30 min at −78° C. ZnCl₂ (1.0 M in Et₂O, 6.80 mL) was added, and the mixture was stirred for 30 min at −78° C. before warming to 0° C. After 30 min, CuI (1.29 g) was added, and the resulting suspension was stirred for 30 min at 0° C. Next, a solution of 4-(3-chlorocarbonyl-propyl)-piperidine-1-carboxylic acid tert-butyl ester (1.96 g) in THF (10 mL) was added via cannula, and the resulting mixture was stirred at 0° C. for 1 h. The mixture was diluted with EtOAc (400 mL) and washed with H₂O (1×80 mL). The organic layer was dried (Na₂SO₄) and concentrated. Chromatographic purification (EtOAc/hexanes) afforded the title compound as a pale yellow solid (1.54 g, 77%). HPLC: Rₜ=7.0 min. MS (ESI): mass calcd. for C₁₇H₂₆N₂O₄, 322.19; m/z found, 345.3 [M+Na]⁺. ¹H NMR (CDCl₃): 7.82 (br s, 1H), 7.33 (br s, 1H), 4.07 (br m, 2H), 3.07 (t, J=7.5, 2H), 2.67 (br m, 2H), 1.78 (m, 2H), 1.67 (m, 2H), 1.45 (s, 9H), 1.44-1.28 (m, 3H), 1.09 (m, 2H).

### Alternative Method for Making the Product of Example 1

Step A. 4-(3-Ethoxycarbonyl-propyl)-piperidine-1-carboxylic acid tert-butyl ester. To a 5 L, jacketed reactor affixed with an overhead mechanical stirrer, N_{2(g)} inlet, and thermocouple were added ethanol (1.4 L) and then acetyl chloride (6.95 mL, 97.7 mmol). After stirring for 5 minutes, piperidine butyric acid.HCl (70.3 g, 337 mmol) was added and the mixture was heated at reflux for 2.5 h. The reaction mixture was cooled to 40° C., and NaHCO₃ (70.8 g, 842 mmol) was added. Upon additional cooling to rt, di-tert-butyl dicarbonate (73.5 mL, 320 mmol) was added followed by water (980 mL). The mixture was stirred overnight and then concentrated to 1,075 g. The concentrate was slurried with CH₂Cl₂ (1.5 L) and treated slowly with 1 N HCl (450 mL) until pH ˜3. The layers were quickly mixed and separated and the aqueous layer was extracted a second time with CH₂Cl₂. The combined organic layers were dried (MgSO₄), filtered, and concentrated to a slightly off-color, clear oil (94.7 g, 97%). ¹H NMR (CDCl₃): 4.13 (q, J=7.2, 2H), 4.07 (br s, 2H), 2.67 (br t, J=12.2, 2H), 2.28 (t, J=7.3, 2H), 1.69-1.60 (m, 4H), 1.45 (s, 9H), 1.42-1.32 (m, 1H), 1.26 (t, J=7.2, 3H), 1.30-1.22 (m, 2H), 1.15-1.01 (m, 2H).

Step B. 4-[3-(Methoxy-methyl-carbamoyl)-propyl]-piperidine-1-carboxylic acid tert-butyl ester. In a 5 L reactor, equipped as in Step A, were slurried the 4-(3-ethoxycarbonyl-propyl)-piperidine-1-carboxylic acid tert-butyl ester (94.6 g, 316 mmol) and N,O-dimethylhydroxylamine.HCl (47.8 g, 490 mmol) in THF (1.05 L). To this slurry was added i-PrMgCl (2.0 M in Et₂O, 474 mL, 948 mmol) over 1.5 h such that the internal temperature remained below −5° C. After addition was complete, the mixture was stirred for 40 min, and then 13% NH₄Cl_{(aq)} (1.5 L) was added. After stirring an additional 15 min and warming to rt, the layers were separated. The aqueous layer was extracted with MTBE (1.5 L). The combined organic layers were dried (MgSO₄), filtered, and concentrated to a slightly yellow oil (97.2 g, 98%). ¹H NMR (CDCl₃): 4.06 (br s, 2H), 3.68 (s, 3H), 3.18 (s, 3H), 2.67 (br t, J=12.1, 2H), 2.41 (t; J=7.6, 2H), 1.71-1.60 (m, 4H), 1.45 (s, 9H), 1.45-1.34 (m, 1H), 1.32-1.24 (m, 2H), 1.15-1.02 (m, 2H).

Step C. To a 5 L reactor, equipped as in Step A, were added THF (1.5 L) and oxazole (25.6 g, 371 mmol). The mixture was cooled to an internal temperature of −15° C. and i-PrMgCl (2 M in Et₂O, 186 mL, 371 mmol) was added over 20 min, maintaining an internal temperature below −10° C. The mixture was stirred for 40 min and then 4-[3-(methoxy-methyl-carbamoyl)-propyl]-piperidine-1-carboxylic acid tert-butyl ester (97.2 g, 309 mmol) was added in THF (0.55 L) through a cannula over 8 min. The solution was warmed to 28° C. and was allowed to stir for 14 h. The reaction was quenched with 13% NH₄Cl_{(aq)} (2 L), and the layers were vigorously mixed and separated. The aqueous layer was extracted with MTBE (1.5 L) and the combined organic layers were dried (MgSO₄), filtered, and concentrated. The resulting crude oil was purified by elution through a plug of SiO₂ with 25% EtOAc/hexanes (6 L) to give a 77% yield of the desired compound (98% purity by ¹H NMR analysis). Subsequent trituration in pentane to provide the title compound as a white solid. (67.37 g, 68%). MS (ESI): mass calcd. for C₁₇H₂₆N₂O₄, 322.19; m/z found, 345.0 [M+Na]⁺. ¹H NMR (CDCl₃): 7.82 (d, J=0.5, 1H), 7.33 (d, J=0.5, 1H), 4.07 (br s, 2H), 3.07 (t, J=7.4, 2H), 2.67 (br t, J=12.1, 2H), 1.83-1.74 (m, 2H), 1.67 (br d, J=13.1, 2H), 1.45 (s, 9H), 1.46-1.36 (m, 1H), 1.36-1.28 (m, 2H), 1.15-1.03 (m, 2H).

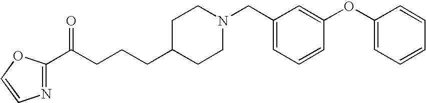

### Example 2

### 1-Oxazol-2-yl-4-[1-(3-phenoxy-benzyl)-piperidin-4-yl]-butan-1-one

Step A. 1-Oxazol-2-yl-4-piperidin-4-yl-butan-1-one hydrochloride. A suspension of 4-(4-oxazol-2-yl-4-oxo-butyl)-piperidine-1-carboxylic acid tert-butyl ester (1.15 g) in HCl (2.0 M in Et₂O, 15 mL) was stirred for 24 h. The suspension was concentrated to afford the title compound as a white solid (901 mg, 98%). HPLC: Rₜ=3.8 min. MS (ESI): mass calcd. for C₁₂H₁₈N₂O₂, 222.14; m/z found, 223.3 [M+H]⁺. ¹H NMR (CD₃OD): 8.12 (d, J=0.7, 1H), 7.42 (d, J=0.7, 1H), 3.37 (br m, 2H), 3.09 (t, J=7.0, 2H), 2.97 (br m, 2H), 1.97 (m, 2H), 1.78 (m, 2H), 1.65 (m, 1H), 1.44-1.30 (m, 4H).

Step B. To a stirred solution of 1-oxazol-2-yl-4-piperidin-4-yl-butan-1-one hydrochloride (202 mg), NEt₃ (120 μL), and 3-phenoxybenzaldehyde (150 μL) in CH₂Cl₂ (4.0 mL) was added NaB(OAc)₃H (180 mg). After 24 h, the mixture was filtered through a short pad of SiO₂ (MeOH/CH₂Cl₂) and the filtrate was concentrated. Chromatographic purification (MeOH/CH₂Cl₂) afforded the title compound as a colorless oil (205 mg, 65%). HPLC: Rₜ=4.9 min. MS (ESI): mass calcd. for C₂₅H₂₈N₂O₃, 404.21; m/z found, 405.3 [M+H]⁺. ¹H NMR (CDCl₃): 7.82 (d, J=0.6, 1H), 7.38-7.29 (m, 3H), 7.32 (d, J=0.6, 1H), 7.23 (m, 1H), 7.12 (m, 1H), 7.05-6.98 (m, 3H), 6.94 (m, 1H), 3.76 (br m, 2H), 3.12 (br m, 2H), 3.05 (t, J=7.3, 2H), 2.26 (m, 2H), 1.75 (m, 4H), 1.57 (m, 2H), 1.36 (m, 3H).

Examples 3-24 were prepared and purified using methods analogous to those described in Example 2, substituting the appropriate aldehyde reagents.

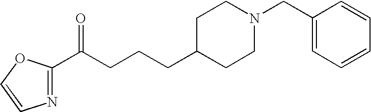

### Example 3

### 4-(1-Benzyl-piperidin-4-yl)-1-oxazol-2-yl-butan-1-one

HPLC: Rₜ=4.1 min. MS (ESI): mass calcd. for C₁₉H₂₄N₂O₂, 312.18; m/z found, 313.3 [M+H]⁺. ¹H NMR (CD₃OD): 8.10 (d, J=2.4, 1H), 7.40 (d, J=2.4, 1H), 7.38-7.30 (m, 5H), 3.75 (br m, 2H), 3.05 (br m, 4H), 2.31 (m, 2H), 1.76 (m, 4H), 1.48-1.20 (m, 5H).

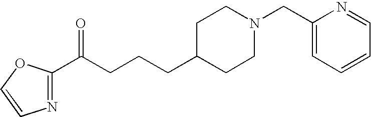

### Example 4

### 1-Oxazol-2-yl-4-(1-pyridin-2-ylmethyl-piperidin-4-yl)-butan-1-one

HPLC: Rₜ=4.1 min. MS (ESI): mass calcd. for C₁₈H₂₃N₃O₂, 313.18; m/z found, 314.3 [M+H]⁺. ¹H NMR (CD₃OD): 8.49 (br m, 1H), 8.11 (d, J=1.2, 1H), 7.84 (br m, 1H), 7.52 (br m, 1H), 7.40 (d, J=1.2, 1H), 7.35 (br m, 1H), 3.75 (br m, 2H), 3.00 (br m, 4H), 2.22 (m, 2H), 1.72 (m, 4H), 1.31 (m, 5H).

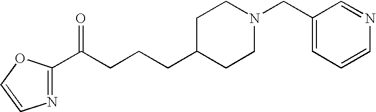

### Example 5

### 1-Oxazol-2-yl-4-(1-pyridin-3-ylmethyl-piperidin-4-yl)-butan-1-one

HPLC: Rₜ=3.7 min. MS (ESI): mass calcd. for C₁₈H₂₃N₃O₂, 313.18; m/z found, 314.3 [M+H]⁺. ¹H NMR (CD₃OD): 8.48 (br m, 2H), 8.10 (d, J=1.3, 1H), 7.83 (br m, 1H), 7.42 (br m, 1H), 7.40 (d, J=1.3, 1H), 3.57 (br m, 2H), 3.04 (t, J=7.4, 2H), 2.88 (br m, 2H), 2.22 (m, 2H), 1.72 (m, 4H), 1.31 (m, 5H).

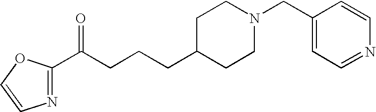

### Example 6

### 1-Oxazol-2-yl-4-(1-pyridin-4-ylmethyl-piperidin-4-yl)-butan-1-one

HPLC: Rₜ=3.6 min. MS (ESI): mass calcd. for C₁₈H₂₃N₃O₂, 313.18; m/z found, 314.3 [M+H]⁺. ¹H NMR (CD₃OD): 8.47 (br m, 2H), 8.11 (d, J=1.4, 1H), 7.42 (br m, 2H), 7.40 (d, J=1.4, 1H), 3.55 (br m, 2H), 3.04 (t, J=7.3, 2H), 2.86 (br m, 2H), 2.04 (m, 2H), 1.73 (m, 4H), 1.30 (m, 5H).

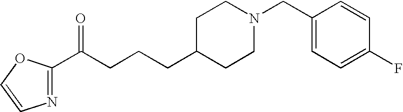

### Example 7

### 4-[1-(4-Fluoro-benzyl)-piperidin-4-yl]-1-oxazol-2-yl-butan-1-one

HPLC: Rₜ=4.4 min MS (ESI): mass calcd. for C₁₉H₂₃FN₂O₂, 330.17; m/z found, 331.3 [M+H]⁺. ¹H NMR (CDCl₃): 7.81 (d, J=0.8, 1H), 7.32 (d, J=0.8, 1H), 7.26 (m, 2H), 6.99 (m, 2H), 3.44 (br s, 2H), 3.06 (t, J=7.4, 2H), 2.84 (br m, 2H), 1.91 (m, 2H), 1.76 (m, 2H), 1.66 (m, 2H), 1.28 (m, 5H).

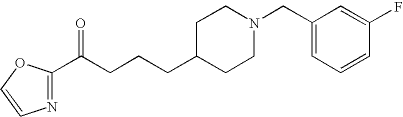

### Example 8

### 4-[1-(3-Fluoro-benzyl)-piperidin-4-yl]-1-oxazol-2-yl-butan-1-one

HPLC: Rₜ=4.4 min. MS (ESI): mass calcd. for C₁₉H₂₃FN₂O₂, 330.17; m/z found, 331.3 [M+H]⁺. ¹H NMR (CDCl₃): 7.82 (d, J=0.8, 1H), 7.32 (d, J=0.8, 1H), 7.25 (m, 1H), 7.07 (m, 2H), 6.94 (m, 1H), 3.47 (br s, 2H), 3.06 (t, J=7.4, 2H), 2.85 (br m, 2H), 1.95 (br m, 2H), 1.76 (m, 2H), 1.67 (m, 2H), 1.29 (m, 5H).

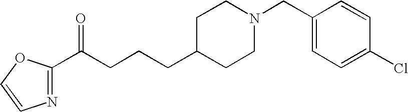

### Example 9

### 4-[1-(4-Chloro-benzyl)-piperidin-4-yl]-1-oxazol-2-yl-butan-1-one

HPLC: Rₜ=4.6 min. MS (ESI): mass calcd. for C₁₉H₂₃ClN₂O₂, 346.14; m/z found, 347.3 [M+H]⁺. ¹H NMR (CD₃OD): 8.10 (d, J=0.9, 1H), 7.40 (d, J=0.9, 1H), 7.33 (m, 4H), 3.56 (br s, 2H), 3.04 (t, J=7.3, 2H), 2.92 (br m, 2H), 2.09 (m, 2H), 1.72 (m, 4H), 1.28 (m, 5H).

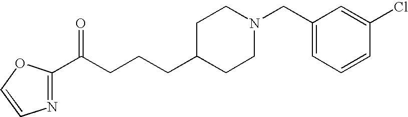

### Example 10

### 4-[1-(3-Chloro-benzyl)-piperidin-4-yl]-1-oxazol-2-yl-butan-1-one

HPLC: Rₜ=4.6 min. MS (ESI): mass calcd. for C₁₉H₂₃ClN₂O₂, 346.14; m/z found, 347.3 [M+H]⁺. ¹H NMR (CDCl₃): 7.81 (d, J=0.8, 1H), 7.33 (d, J=0.8, 1H), 7.22 (m, 4H), 3.44 (br m, 2H), 3.06 (t, J=7.4, 2H), 2.84 (br m, 2H), 1.93 (m, 2H), 1.77 (m, 2H), 1.66 (m, 2H), 1.29 (m, 5H).

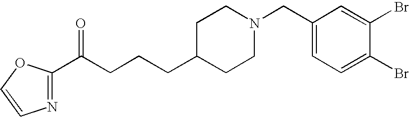

### Example 11

### 4-[1-(3,4-Dibromo-benzyl)-piperidin-4-yl]-1-oxazol-2-yl-butan-1-one

HPLC: Rₜ=4.8 min. MS (ESI): mass calcd. for C₁₉H₂₂Br₂N₂O₂, 468.00; m/z found, 469.1 [M+H]⁺. ¹H NMR (CD₃OD): 8.10 (d, J=0.8, 1H), 7.68 (d, J=2.0, 1H), 7.63 (d, J=8.4, 1H), 7.40 (d, J=0.8, 1H), 7.22 (dd, J=8.1, 2.0, 1H), 3.49 (br s, 2H), 3.04 (t, J=7.3, 2H), 2.88 (br m, 2H), 2.05 (m, 2H), 1.72 (m, 4H), 1.28 (m, 5H).

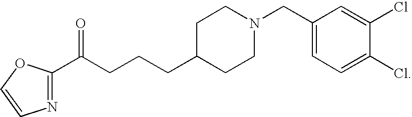

### Example 12

### 4-[1-(3,4-Dichloro-benzyl)-piperidin-4-yl]-1-oxazol-2-yl-butan-1-one

HPLC: Rₜ=4.8 min. MS (ESI): mass calcd. for C₁₉H₂₂Cl₂N₂O₂, 380.11; m/z found, 381.2 [M+H]⁺. ¹H NMR (CDCl₃): 7.82 (d, J=0.8, 1H), 7.42 (d, J=2.0, 1H), 7.36 (d, J=8.3, 1H), 7.32 (d, J=0.8, 1H), 7.15 (m, 1H), 3.41 (br s, 2H), 3.06 (t, J=7.3, 2H), 2.82 (br m, 2H), 1.93 (m, 2H), 1.76 (m, 2H), 1.67 (m, 2H), 1.29 (m, 5H).

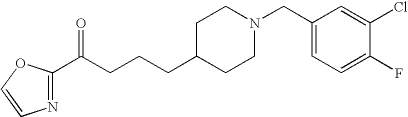

### Example 13

### 4-[1-(3-Chloro-4-fluoro-benzyl)-piperidin-4-yl]-1-oxazol-2-yl-butan-1-one

HPLC: Rₜ=4.6 min. MS (ESI): mass calcd. for C₁₉H₂₂ClFN₂O₂, 364.14; m/z found, 365.2 [M+H]⁺. ¹H NMR (CDCl₃): 7.81 (d, J=0.8, 1H), 7.37 (dd, J=7.0, 2.0, 1H), 7.33 (d, J=0.8, 1H), 7.16 (m, 1H), 7.06 (m, 1H), 3.40 (br m, 2H), 3.06 (t, J=7.3, 2H), 2.82 (br m, 2H), 1.92 (m, 2H), 1.76 (m, 2H), 1.67 (m, 2H), 1.29 (m, 5H).

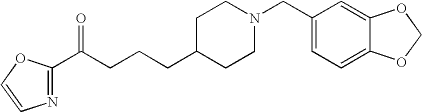

### Example 14

### 4-(1-Benzo[1,3]dioxol-5-ylmethyl-Piperidin-4-yl)-1-oxazol-2-yl-butan-1-one

HPLC: Rₜ=4.4 min. MS (ESI): mass calcd. for C₂₀H₂₄N₂O₄, 356.17; m/z found, 357.3 [M+H]⁺. ¹H NMR (CD₃OD): 8.11 (d, J=0.8, 1H), 7.41 (d, J=0.8, 1H), 6.91 (d, J=1.5, 1H), 6.83 (m, 2H), 5.96 (br s, 2H), 3.77 (br s, 2H), 3.13 (br m, 2H), 3.05 (t, J=7.3, 2H), 2.42 (br m, 2H), 1.83 (m, 2H), 1.74 (br m, 2H), 1.44 (br m, 1H), 1.33 (m, 4H).

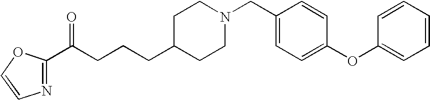

### Example 15

### 1-Oxazol-2-yl-4-[1-(4-phenoxy-benzyl)-piperidin-4-yl]-butan-1-one

HPLC: Rₜ=5.0 min. MS (ESI): mass calcd. for C₂₅H₂₈N₂O₃, 404.21; m/z found, 405.3 [M+H]⁺. ¹H NMR (CD₃OD): 8.11 (d, J=0.8, 1H), 7.38 (m, 5H), 7.14 (m, 1H), 6.99 (m, 4H), 3.86 (br s, 2H), 3.17 (br m, 2H), 3.06 (t, J=7.3, 2H), 2.48 (br m, 2H), 1.85 (m, 2H), 1.74 (br m, 2H), 1.46 (br m, 1H), 1.36 (m, 4H).

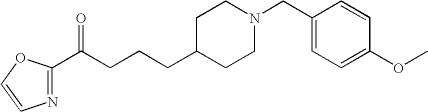

### Example 16

### 4-[1-(4-Methoxy-benzyl)-piperidin-4-yl]-1-oxazol-2-yl-butan-1-one

HPLC: Rₜ=4.4 min. MS (ESI): mass calcd. for C₂₀H₂₆N₂O₃, 342.19; m/z found, 343.4 [M+H]⁺. ¹H NMR (CD₃OD): 8.11 (d, J=0.8, 1H), 7.41 (d, J=0.8, 1H), 7.33 (m, 2H), 6.95 (m, 2H), 3.87 (br s, 2H), 3.80 (s, 3H), 3.18 (br m, 2H), 3.05 (t, J=7.3, 2H), 2.52 (br m, 2H), 1.85 (m, 2H), 1.74 (br m, 2H), 1.47 (br m, 1H), 1.35 (m, 4H).

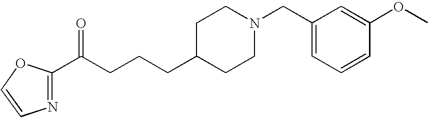

### Example 17

### 4-[1-(3-Methoxy-benzyl)-piperidin-4-yl]-1-oxazol-2-yl-butan-1-one

HPLC: Rₜ=4.4 min. MS (ESI): mass calcd. for C₂₀H₂₆N₂O₃, 342.19; m/z found, 343.4 [M+H]⁺. ¹H NMR (CD₃OD): 8.11 (d, J=0.8, 1H), 7.41 (d, J=0.8, 1H), 7.26 (t, J=7.8, 1H), 6.97 (m, 1H), 6.93 (m, 1H), 6.88 (m, 1H), 3.80 (s, 3H), 3.69 (br s, 2H), 3.05 (m, 4H), 2.29 (br m, 2H), 1.75 (br m, 4H), 1.44-1.20 (br m, 5H).

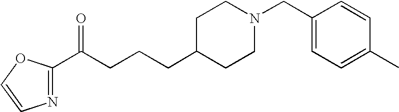

### Example 18

### 4-[1-(4-Methyl-benzyl)-piperidin-4-yl]-1-oxazol-2-yl-butan-1-one

HPLC: Rₜ=4.6 min. MS (ESI): mass calcd. for C₂₀H₂₆N₂O₂, 326.20; m/z found, 327.3 [M+H]⁺. ¹H NMR (CDCl₃): 7.81 (d, J=0.8, 1H), 7.32 (d, J=0.8, 1H), 7.16 (m, 4H), 3.45 (br s, 2H), 3.05 (t, J=7.3, 2H), 2.87 (br m, 2H), 2.32 (s, 3H), 1.91 (m, 2H), 1.76 (br m, 2H), 1.62 (br m, 2H), 1.28 (m, 5H).

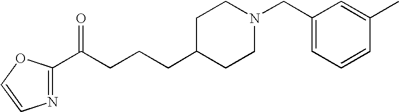

### Example 19

### 4-[1-(3-Methyl-benzyl)-piperidin-4-yl]-1-oxazol-2-yl-butan-1-one

HPLC: Rₜ=4.6 min. MS (ESI): mass calcd. for C₂₀H₂₆N₂O₂, 326.20; m/z found, 327.3 [M+H]⁺. ¹H NMR (CDCl₃): 7.81 (d, J=0.8, 1H), 7.32 (d, J=0.8, 1H), 7.20 (m, 1H), 7.10 (m, 3H), 3.47 (br s, 2H), 3.05 (t, J=7.3, 2H), 2.89 (br m, 2H), 2.34 (s, 3H), 1.94 (m, 2H), 1.76 (br m, 2H), 1.67 (br m, 2H), 1.29 (m, 5H).

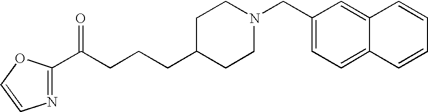

### Example 20

### 4-(1-Naphthalen-2-ylmethyl-piperidin-4-yl)-1-oxazol-2-yl-butan-1-one

HPLC: Rₜ=4.7 min. MS (ESI): mass calcd. for C₂₃H₂₆N₂O₂, 362.20; m/z found, 363.4 [M+H]⁺. ¹H NMR (CD₃OD): 8.11 (d, J=0.8, 1H), 8.00-7.88 (m, 4H), 7.51 (m, 3H), 7.40 (d, J=0.8, 1H), 4.23 (br s, 2H), 3.34 (m, 2H), 3.06 (t, J=7.3, 2H), 2.78 (br m, 2H), 1.91 (br m, 2H), 1.73 (m, 2H), 1.54 (m, 1H), 1.47-1.32 (br m, 4H).

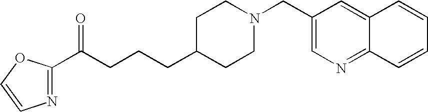

### Example 21

### 1-Oxazol-2-yl-4-(1-quinolin-3-ylmethyl-piperidin-4-yl)-butan-1-one

HPLC: Rₜ=4.0 min. MS (ESI): mass calcd. for C₂₂H₂₅N₃O₂, 363.19; m/z found, 364.3 [M+H]⁺. ¹H NMR (CD₃OD): 8.85 (d, J=2.1, 1H), 8.27 (br s, 1H), 8.10 (d, J=0.8, 1H), 8.02 (d, J=8.4, 1H), 7.94 (d, J=8.1, 1H), 7.76 (m, 1H), 7.62 (m, 1H), 7.40 (d, J=0.8, 1H), 3.75 (br s, 2H), 3.04 (t, J=7.3, 2H), 2.95 (m, 2H), 2.12 (br m, 2H), 1.73 (br m, 3H), 1.61 (m, 1H), 1.35-1.20 (br m, 5H).

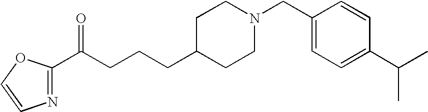

### Example 22

### 4-[1-(4-Isopropyl-benzyl)-piperidin-4-yl]-1-oxazol-2-yl-butan-1-one

HPLC: Rₜ=4.9 min. MS (ESI): mass calcd. for C₂₂H₃₀N₂O₂, 354.23; m/z found, 355.4 [M+H]⁺. ¹H NMR (CD₃OD): 8.11 (d, J=0.8, 1H), 7.40 (br m, 3H), 7.33 (br m, 2H), 3.36 (br m, 2H), 3.07 (t, J=7.3, 2H), 2.94 (septet, J=6.8, 1H), 2.85 (m, 2H), 1.94 (br m, 4H), 1.75 (br m, 2H), 1.58 (m, 1H), 1.40 (br m, 4H), 1.24 (d, J=6.8, 6H).

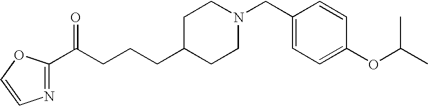

### Example 23

### 4-[1-(4-isopropoxy-benzyl)-piperidin-4-yl]-1-oxazol-2-yl-butan-1-one

HPLC: Rₜ=4.8 min. MS (ESI): mass calcd. for C₂₂H₃₀N₂O₃, 370.23; m/z found, 371.4 [M+H]⁺. ¹H NMR (CD₃OD): 8.11 (d, J=0.8, 1H), 7.41 (d, J=0.8, 1H), 7.33 (br m, 2H), 6.94 (br m, 2H), 4.62 (septet, J=6.1, 1H), 3.96 (br s, 2H), 3.27 (br m, 2H), 3.06 (t, J=7.1, 2H), 2.66 (m, 2H), 1.90 (br m, 2H), 1.75 (br m, 2H), 1.51 (m, 1H), 1.37 (br m, 4H), 1.30 (d, J=6.1, 6H).

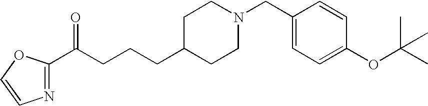

### Example 24

### 4-[1-(4-tert-Butoxy-benzyl)-piperidin-4-yl]-1-oxazol-2-yl-butan-1-one

HPLC: Rₜ=4.8 min. MS (ESI): mass calcd. for C₂₃H₃₂N₂O₃, 384.24; m/z found, 385.4 [M+H]⁺. ¹H NMR(CD₃OD)₃OD): 8.11 (d, J=0.8, 1H), 7.41 (d, J=0.8, 1H), 7.36 (br m, 2H), 7.04 (br m, 2H), 3.99 (br s, 2H), 3.27 (br m, 2H), 3.06 (t, J=7.1, 2H), 2.68 (m, 2H), 1.90 (br m, 2H), 1.74 (br m, 2H), 1.52 (m, 1H), 1.37 (br m, 4H), 1.30 (s, 9H).

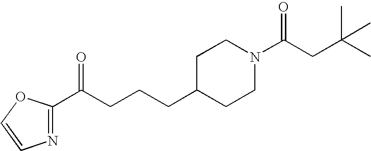

### Example 25

### 3,3-Dimethyl-1-[4-(4-oxazol-2-yl-4-oxo-butyl)-piperidin-1-yl]-butan-1-one

To a stirred solution of 1-oxazol-2-yl-4-piperidin-4-yl-butan-1-one hydrochloride (236 mg) and NEt₃ (380 μL) in CH₂Cl₂ (4.0 mL) was added tert-butylacetyl chloride (140 μL). After 30 min, the mixture was diluted with CH₂Cl₂ (80 mL) and washed with H₂O (1×20 mL). The organic layer was dried (Na₂SO₄) and concentrated. Chromatographic purification (EtOAc/hexanes) afforded the title compound as a colorless oil (187 mg, 64%). HPLC: Rₜ=6.3 min. MS (ESI): mass calcd. for C₁₈H₂₈N₂O₃, 320.21; m/z found, 321.4 [M+H]⁺. ¹H NMR (CDCl₃): 7.83 (d, J=0.6, 1H), 7.33 (d, J=0.6, 1H), 4.67 (br m, 2H), 3.93 (br m, 2H), 3.08 (t, J=7.5, 2H), 2.98 (m, 1H), 2.50 (m, 1H), 2.26 (m, 2H), 1.77 (m, 3H), 1.51 (m, 1H), 1.35 (m, 2H), 1.10 (m, 1H), 1.05 (s, 9H).

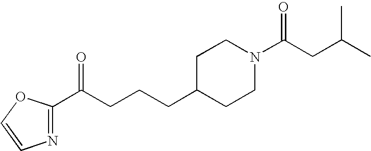

### Example 26

### 3-Methyl-1-[4-(4-oxazol-2-yl-4-oxo-butyl)-piperidin-1-yl]-butan-1-one

The title compound was prepared from 3-methylbutanoyl chloride using methods analogous to those described in Example 25. HPLC: Rₜ=6.0 min. MS (ESI): mass calcd. for C₁₇H₂₆N₂O₃, 306.19; m/z found, 307.4 [M+H]⁺. ¹H NMR (CDCl₃): 7.83 (d, J=0.8, 1H), 7.33 (d, J=0.8, 1H), 4.63 (br m, 1H), 3.86 (br m, 1H), 3.08 (t, J=7.4, 2H), 2.98 (br m, 1H), 2.51 (br m, 1H), 2.21 (d, J=2.7, 1H), 2.20 (d, J=1.8, 1H), 2.10 (m, 2H), 1.77 (br m, 3H), 1.53 (br m, 1H), 1.34 (br m, 2H), 1.09 (br m, 2H), 0.97 (d, J=6.4, 6H).

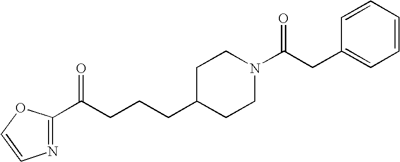

### Example 27

### 1-Oxazol-2-yl-4-(1-phenylacetyl-piperidin-4-yl)-butan-1-one

To a mixture of 1-oxazol-2-yl-4-piperidin-4-yl-butan-1-one hydrochloride (224 mg) and satd. aq. NaHCO₃ (1.0 mL) in EtOAc (4.0 mL) was added phenylacetyl chloride (160 μL). After 18 h, the mixture was diluted with EtOAc (40 mL) and washed with H₂O (1×10 mL). The organic layer was dried (Na₂SO₄) and concentrated. Chromatographic purification (EtOAc/hexanes) afforded the title compound as a colorless oil (161 mg, 55%). HPLC: Rₜ=6.0 min. MS (ESI): mass calcd. for C₂₀H₂₄N₂O₃, 340.18; m/z found, 341.3 [M+H]⁺. ¹H NMR (CDCl₃): 7.82 (br s, 1H), 7.34-7.21 (m, 5H), 7.33 (br s, 1H), 4.63 (br m, 1H), 3.85 (br m, 1H), 3.73 (s, 2H), 3.05 (t, J=7.4, 2H), 2.92 (m, 1H), 2.55 (m, 1H), 1.74 (m, 3H), 1.61 (m, 1H), 1.46 (m, 1H), 1.28 (m, 2H), 1.07 (m, 1H), 0.85 (m, 1H).

Examples 28-29 were prepared and purified using methods analogous to those described Example 27, substituting the appropriate acid chloride reagents.

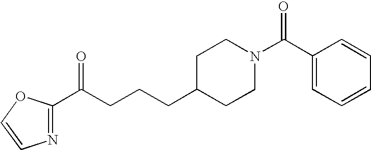

### Example 28

### 4-(1-Benzoyl-piperidin-4-yl)-1-oxazol-2-yl-butan-1-one

HPLC: Rₜ=5.9 min. MS (ESI): mass calcd. for C₁₉H₂₂N₂O₃, 326.16; m/z found, 327.3 [M+H]⁺. ¹H NMR (CDCl₃): 7.83 (br s, 1H), 7.39 (m, 5H), 7.33 (br s, 1H), 4.71 (br m, 1H), 3.74 (br m, 1H), 3.08 (t, J=7.4, 2H), 2.97 (br m, 1H), 2.75 (m, 1H), 1.88-1.52 (m, 5H), 1.37 (m, 2H), 1.30-1.02 (m, 2H).

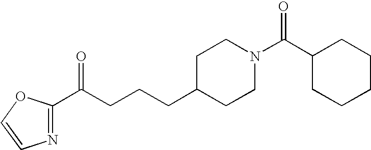

### Example 29

### 4-(1-Cyclohexanecarbonyl-piperidin-4-yl)-1-oxazol-2-yl-butan-1-one

HPLC: Rₜ=6.5 min. MS (ESI): mass calcd. for C₁₉H₂₈N₂O₃, 332.21; m/z found, 333.4 [M+H]⁺. ¹H NMR (CDCl₃): 7.83 (d, J=0.8, 1H), 7.33 (d, J=0.8, 1H), 4.62 (br m, 1H), 3.91 (br m, 1H), 3.08 (t, J=7.4, 2H), 2.97 (br m, 1H), 2.47 (br m, 3H), 1.84-1.60 (br m, 7H), 1.68 (br m, 2H), 1.52 (br m, 2H), 1.30 (br m, 4H), 1.08 (br m, 3H).

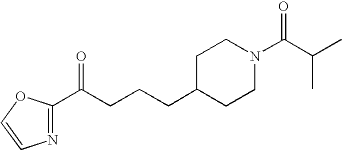

### Example 30

### 4-(1-Isobutyryl-piperidin-4-yl)-1-oxazol-2-yl-butan-1-one

To a stirred solution of 1-oxazol-2-yl-4-piperidin-4-yl-butan-1-one hydrochloride (218 mg) and pyridine (204 μL) in CH₂Cl₂ (4.0 mL) was added isobutyryl chloride (98 μL). After 30 min, the mixture was diluted with CH₂Cl₂ (30 mL) and washed with H₂O (1×20 mL). The organic layer was dried (Na₂SO₄) and concentrated. Chromatographic purification (EtOAc/hexanes) afforded the title compound as a white solid (212 mg, 86%). HPLC: Rₜ=5.6 min. MS (ESI): mass calcd. for C₁₆H₂₄N₂O₃, 292.18; m/z found, 293.3 [M+H]⁺. ¹H NMR (CDCl₃): 7.83 (d, J=0.6, 1H), 7.33 (d, J=0.6, 1H), 4.67 (br m, 2H), 3.93 (br m, 2H), 3.08 (t, J=7.5, 2H), 2.98 (m, 1H), 2.50 (m, 1H), 2.26 (m, 2H), 1.77 (m, 3H), 1.51 (m, 1H), 1.35 (m, 2H), 1.05 (s, 9H). 1.10 (m, 1H).

Examples 31-36 were prepared and purified using methods analogous to those described in Example 30, substituting the appropriate acid chloride reagents.

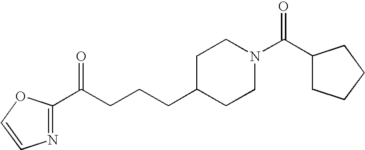

### Example 31

### 4-(1-Cyclopentanecarbonyl-piperidin-4-yl)-1-oxazol-2-yl-butan-1-one

HPLC: Rₜ=6.3 min. MS (ESI): mass calcd. for C₁₈H₂₆N₂O₃, 318.19; m/z found, 319.4 [M+H]⁺. ¹H NMR (CDCl₃): 7.83 (d, J=0.8, 1H), 7.33 (d, J=0.8, 1H), 4.62 (br m, 1H), 3.96 (br m, 1H), 3.08 (t, J=7.4, 2H), 2.97 (br m, 1H), 2.88 (quint, J=7.8, 1H), 2.52 (br m, 1H), 1.86-1.66 (br m, 7H), 1.56 (br m, 4H), 1.34 (br m, 4H), 1.09 (br m, 2H).

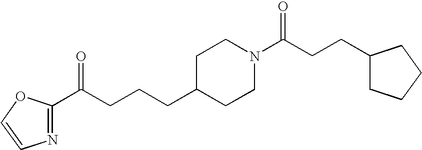

### Example 32

### 4-[1-(3-Cyclopentyl-propionyl)-piperidin-4-yl]-1-oxazol-2-yl-butan-1-one

HPLC: Rₜ=7.0 min. MS (ESI): mass calcd. for C₂₀H₃₀N₂O₃, 346.23; m/z found, 347.4 [M+H]⁺. ¹H NMR (CDCl₃): 7.83 (d, J=0.8, 1H), 7.33 (d, J=0.8, 1H), 4.61 (br m, 1H), 3.84 (br m, 1H), 3.08 (t, J=7.4, 2H), 2.98 (br m, 1H), 2.50 (br m, 1H), 2.35 (m, 3H), 1.81-1.39 (br m, 11H), 1.34 (br m, 2H), 1.10 (br m, 6H).

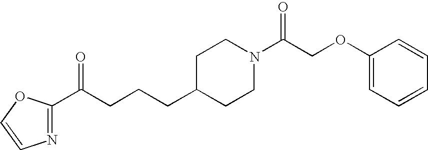

### Example 33

### 1-Oxazol-2-yl-4-[1-(2-phenoxy-acetyl)-piperidin-4-yl]-butan-1-one

HPLC: Rₜ=6.1 min. MS (ESI): mass calcd. for C₂₀H₂₄N₂O₄, 356.17; m/z found, 357.3 [M+H]⁺. ¹H NMR (CDCl₃): 7.83 (d, J=0.8, 1H), 7.33 (d, J=0.8, 1H), 7.32-7.27 (m, 2H), 6.96 (m, 3H), 4.56 (m, 1H), 3.99 (br m, 1H), 3.07 (t, J=7.4, 2H), 3.02 (br m, 2H), 2.60 (br m, 2H), 1.76 (br m, 4H), 1.54 (br m, 1H), 1.33 (br m, 2H), 1.12 (br m, 2H).

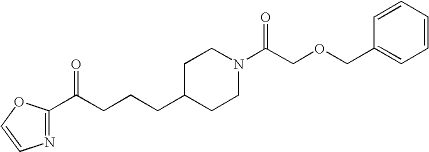

### Example 34

### 4-[1-(2-Benzyloxy-acetyl)-piperidin-4-yl]-1-oxazol-2-yl-butan-1-one

HPLC: Rₜ=6.1 min. MS (ESI): mass calcd. for C₂₁H₂₆N₂O₄, 370.19; m/z found, 371.3 [M+H]⁺. ¹H NMR (CDCl₃): 7.83 (d, J=0.8, 1H), 7.40-7.28 (m, 6H), 4.58 (m, 3H), 4.17 (br m, 2H), 3.86 (m, 1H), 3.07 (t, J=7.4, 2H), 2.95 (br m, 1H), 2.57 (br m, 1H), 1.76 (br m, 4H), 1.52 (br m, 1H), 1.33 (br m, 2H), 1.10 (br m, 2H).

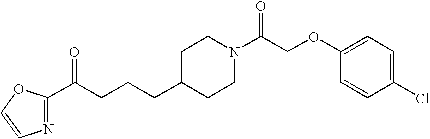

### Example 35

### 4-{1-[2-(4-Chloro-phenoxy)-acetyl]-piperidin-4-yl}-1-oxazol-2-yl-butan-1-one

HPLC: Rₜ=6.4 min. MS (ESI): mass calcd. for C₂₀H₂₃N₂O₄Cl, 390.13; m/z found, 391.3 [M+H]⁺. ¹H NMR (CDCl₃): 7.82 (d, J=0.8, 1H), 7.33 (d, J=0.8, 1H), 7.24 (m, 2H), 6.88 (m, 2H), 4.65 (m, 2H), 4.54 (m, 1H), 3.94 (br m, 1H), 3.07 (t, J=7.4, 2H), 3.02 (m, 1H), 2.61 (br m, 1H), 1.78 (br m, 4H), 1.54 (br m, 1H), 1.33 (br m, 2H), 1.11 (br m, 2H).

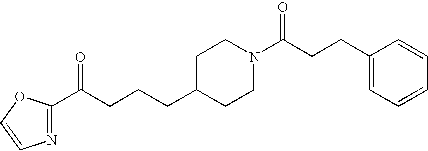

### Example 36

### 1-Oxazol-2-yl-4-[1-(3-phenyl-propionyl)-piperidin-4-yl]-butan-1-one

HPLC: Rₜ=6.3 min. MS (ESI): mass calcd. for C₂₁H₂₆N₂O₃, 354.19; m/z found, 355.3 [M+H]⁺. ¹H NMR (CDCl₃): 7.82 (br s, 1H), 7.33 (br s, 1H), 7.36-7.16 (m, 5H), 4.63 (br m, 1H), 3.77 (br m, 1H), 3.06 (t, J=7.2, 2H), 2.96 (m, 2H), 2.88 (dd, J=7.1, 2.5, 1H), 2.69 (t, J=7.8, 1H), 2.62 (m, 2H), 2.51 (m, 1H), 1.75 (m, 1H), 1.68 (m, 2H), 1.49 (m, 1H), 1.31 (m, 2H), 1.06 (m, 1H), 0.93 (m, 1H).

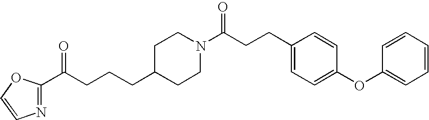

### Example 37

### 1-Oxazol-2-yl-4-{1-[3-(4-phenoxy-phenyl)-propionyl]-piperidin-4-yl}-butan-1-one

To a stirred solution of 1-oxazol-2-yl-4-piperidin-4-yl-butan-1-one hydrochloride (205 mg), 1-(3-dimethylaminopropyl)-3-ethylcarbodiimide hydrochloride (212 mg), and 3-(4-phenoxy-phenyl)-propionic acid (230 mg) in CH₂Cl₂ (8.0 mL) was added NEt₃ (440 μL). After 24 h, the mixture was diluted with CH₂Cl₂ (50 mL) and washed with H₂O (1×30 mL). The organic layer was dried (Na₂SO₄) and concentrated. Chromatographic purification (EtOAc/hexanes) afforded the title compound as a colorless oil (203 mg, 57%). HPLC: Rₜ=7.1 min. MS (ESI): mass calcd. for C₂₇H₃₀N₂O₄, 446.22; m/z found, 447.4 [M+H]⁺. ¹H NMR (CDCl₃): 7.82 (d, J=0.8, 1H), 7.36-7.28 (m, 3H), 7.18 (m, 2H), 7.08 (m, 1H), 7.01-6.91 (m, 4H), 4.63 (br m, 1H), 3.80 (m, 1H), 3.07 (t, J=7.4, 2H), 2.94 (m, 2H), 2.61 (m, 2H), 2.52 (m, 1H), 1.76 (m, 5H), 1.51 (m, 1H), 1.32 (m, 2H), 1.40 (m, 2H).

Examples 38-51 were prepared and purified using methods analogous to those described in Example 37, substituting the appropriate carboxylic acid reagents.

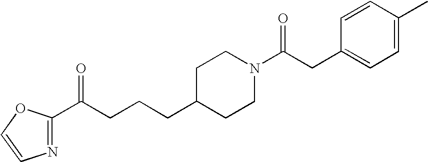

### Example 38

### 1-Oxazol-2-yl-4-[1-(2-p-tolyl-acetyl)-piperidin-4-yl]-butan-1-one

HPLC: Rₜ=6.3 min. MS (ESI): mass calcd. for C₂₁H₂₆N₂O₃, 354.19; m/z found, 355.3 [M+H]⁺. ¹H NMR (CDCl₃): 7.82 (d, J=0.8, 1H), 7.32 (d, J=0.8, 1H), 7.12 (m, 4H), 4.62 (br m, 1H), 3.84 (br m, 1H), 3.68 (s, 2H), 3.05 (t, J=7.4, 2H), 2.91 (br m, 1H), 2.53 (br m, 1H), 2.33 (s, 3H), 1.74 (br m, 3H), 1.61 (br m, 1H), 1.47 (br m, 1H), 1.28 (br m, 2H), 1.09 (br m, 1H), 0.88 (br m, 1H).

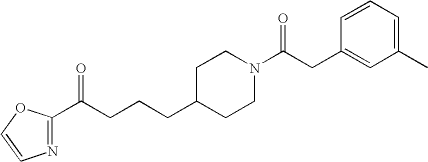

### Example 39

### 1-Oxazol-2-yl-4-[1-(2-m-tolyl-acetyl)-piperidin-4-yl]-butan-1-one

HPLC: Rₜ=6.3 min. MS (ESI): mass calcd. for C₂₁H₂₆N₂O₃, 354.19; m/z found, 355.3 [M+H]⁺. ¹H NMR (CDCl₃): 7.82 (d, J=0.8, 1H), 7.32 (d, J=0.8, 1H), 7.19 (t, J=7.6, 1H), 7.04 (m, 3H), 4.63 (br m, 1H), 3.85 (br m, 1H), 3.69 (s, 2H), 3.05 (t, J=7.4, 2H), 2.91 (br m, 1H), 2.54 (br m, 1H), 2.33 (s, 3H), 1.74 (br m, 3H), 1.62 (br m, 1H), 1.47 (br m, 1H), 1.28 (br m, 2H), 1.07 (br m, 1H), 0.87 (br m, 1H).

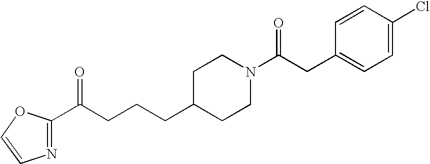

### Example 40

### 4-{1-[2-(4-Chloro-phenyl)-acetyl]-piperidin-4-yl}1-oxazol-2-yl-butan-1-one

HPLC: Rₜ=6.4 min. MS (ESI): mass calcd. for C₂₀H₂₃N₂O₃Cl, 374.14; m/z found, 375.3 [M+H]⁺. ¹H NMR (CDCl₃): 7.82 (d, J=0.8, 1H), 7.33 (d, J=0.8, 1H), 7.29 (m, 2H), 7.18 (m, 2H), 4.61 (br m, 1H), 3.82 (br m, 1H), 3.69 (s, 2H), 3.06 (t, J=7.4, 2H), 2.95 (br m, 1H), 2.56 (br m, 1H), 1.76 (br m, 3H), 1.66 (m, 1H), 1.49 (br m, 1H), 1.30 (br m, 2H), 1.07 (br m, 1H), 0.90 (br m, 1H).

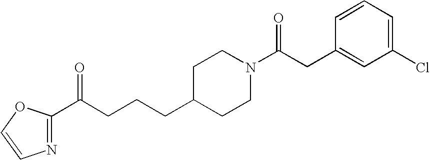

### Example 41

### 4-{1-[2-(3-Chloro-phenyl)-acetyl]-piperidin-4-yl}-1-oxazol-2-yl-butan-1-one

HPLC: Rₜ=6.4 min. MS (ESI): mass calcd. for C₂₀H₂₃N₂O₃Cl, 374.14; m/z found, 375.3 [M+H]⁺. ¹H NMR (CDCl₃): 7.82 (d, J=0.8, 1H), 7.32 (d, J=0.8, 1H), 7.24 (m, 3H), 7.13 (m, 1H), 4.62 (br m, 1H), 3.82 (br m, 1H), 3.69 (s, 2H), 3.06 (t, J=7.4, 2H), 2.96 (br m, 1H), 2.56 (br m, 1H), 1.82-1.63 (br m, 4H), 1.49 (br m, 1H), 1.31 (br m, 2H), 1.09 (br m, 1H), 0.91 (br m, 1H).

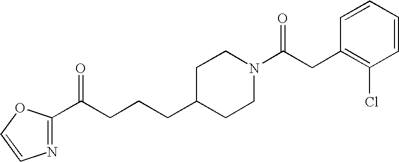

### Example 42

### 4-{1-[2-(2-Chloro-Phenyl)-acetyl]-piperidin-4-yl}-1-oxazol-2-yl-butan-1-one

HPLC: Rₜ=6.4 min. MS (ESI): mass calcd. for C₂₀H₂₃N₂O₃Cl, 374.14; m/z found, 375.2 [M+H]⁺. ¹H NMR (CDCl₃): 7.82 (d, J=0.8, 1H), 7.39-7.16 (m, 5H), 4.64 (br m, 1H), 3.82 (br m, 3H), 3.06 (t, J=7.4, 2H), 2.99 (br m, 1H), 2.59 (br m, 1H), 1.81-1.64 (br m, 5H), 1.32 (br m, 2H), 1.11 (br m, 1H), 0.98 (br m, 1H).

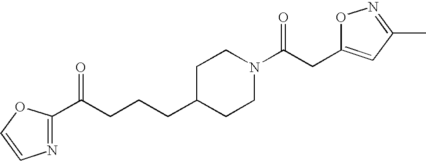

### Example 43

### 4-{1-[2-(3-Methyl-isoxazol-5-yl)-acetyl]-piperidin-4-yl}-1-oxazol-2-yl-butan-1-one

HPLC: Rₜ=6.3 min. MS (ESI): mass calcd. for C₁₈H₂₃N₃O₄, 345.17; m/z found, 346.3 [M+H]⁺. ¹H NMR (CDCl₃): 7.83 (d, J=0.8, 1H), 7.33 (d, J=0.8, 1H), 6.07 (s, 1H), 4.59 (br m, 1H), 3.88 (br m, 1H), 3.82 (s, 2H), 3.07 (t, J=7.4, 2H), 2.59 (br m, 2H), 2.28 (s, 3H), 1.78 (br m, 5H), 1.34 (br m, 2H), 1.10 (br m, 2H).

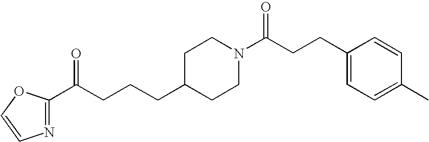

### Example 44

### 1-Oxazol-2-yl-4-[1-(3-p-tolyl-propionyl)-piperidin-4-yl]-butan-1-one

HPLC: Rₜ=6.6 min. MS (ESI): mass calcd. for C₂₂H₂₈N₂O₃, 368.21; m/z found, 369.3 [M+H]⁺. ¹H NMR (CDCl₃): 7.83 (d, J=0.8, 1H), 7.33 (d, J=0.8, 1H), 7.10 (m, 4H), 4.62 (br m, 1H), 3.77 (br m, 1H), 3.06 (t, J=7.4, 2H), 2.91 (m, 3H), 2.59 (br m, 2H), 2.51 (m, 1H), 2.31 (s, 3H), 1.76 (br m, 2H), 1.68 (br m, 2H), 1.49 (br m, 1H), 1.30 (br m, 2H), 1.06 (br m, 1H), 0.93 (br m, 1H).

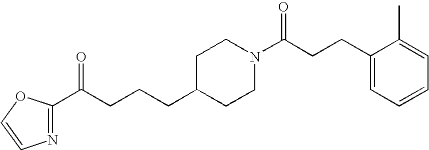

### Example 45

### 1-Oxazol-2-yl-4-[1-(3-o-tolyl-propionyl)-piperidin-4-yl]-butan-1-one

HPLC: Rₜ=6.6 min. MS (ESI): mass calcd. for C₂₂H₂₈N₂O₃, 368.21; m/z found, 369.3 [M+H]⁺. ¹H NMR (CDCl₃): 7.82 (d, J=0.8, 1H), 7.33 (d, J=0.8, 1H), 7.13 (m, 4H), 4.64 (br m, 1H), 3.77 (br m, 1H), 3.07 (t, J=7.4, 2H), 2.96 (m, 2H), 2.89 (dd, J=13.1, 2.8, 1H), 2.56 (br m, 2H), 2.51 (dd, J=12.8, 2.8, 1H), 2.33 (s, 3H), 1.77 (br m, 3H), 1.68 (br m, 1H), 1.49 (br m, 1H), 1.31 (br m, 2H), 1.07 (br m, 1H), 0.95 (br m, 1H).

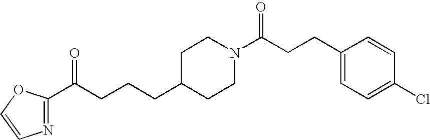

### Example 46

### 4-{1-[3-(4-Chloro-phenyl)-propionyl]-piperidin-4-yl}-1-oxazol-2-yl-butan-1-one

HPLC: Rₜ=6.1 min. MS (ESI): mass calcd. for C₂₁H₂₅ClN₂O₃, 388.16; m/z found, 389.3 [M+H]⁺. ¹H NMR (CDCl₃): 7.81 (d, J=0.8, 1H), 7.32 (d, J=0.8, 1H), 7.23 (m, 2H), 7.14 (m, 2H), 4.59 (br m, 1H), 3.76 (br m, 1H), 3.06 (t, J=7.4, 2H), 2.91 (m, 2H), 2.57 (br m, 1H), 2.51 (m, 1H), 1.73 (br m, 4H), 1.49 (br m, 1H), 1.31 (br m, 4H), 1.05 (br m, 1H), 0.93 (br m, 1H).

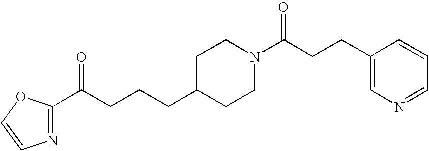

### Example 47

### 1-Oxazol-2-yl-4-[1-(3-pyridin-3-yl-propionyl)-piperidin-4-yl]-butan-1-one

HPLC: Rₜ=4.1 min. MS (ESI): mass calcd. for C₂₀H₂₅N₃O₃, 355.19; m/z found, 356.3 [M+H]⁺. ¹H NMR (CDCl₃): 8.49 (d, J=1.4, 1H), 8.46 (dd, J=4.7, 1.6, 1H), 7.82 (d, J=0.8, 1H), 7.56 (m, 1H), 7.33 (d, J=0.8, 1H), 7.21 (m, 1H), 4.61 (br m, 1H), 3.78 (br m, 1H), 3.07 (t, J=7.4, 2H), 2.97 (m, 2H), 2.92 (dd, J=12.8, 2.7, 1H), 2.62 (dd, J=8.3, 6.8, 2H), 2.53 (m, 1H), 1.75 (br m, 4H), 1.50 (br m, 1H), 1.31 (br m, 2H), 1.03 (br m, 2H).

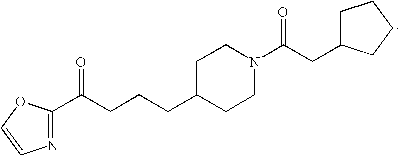

### Example 48

### 4-[1-(2-Cyclopentyl-acetyl)-piperidin-4-yl]-1-oxazol-2-yl-butan-1-one

HPLC: Rₜ=6.5 min. MS (ESI): mass calcd. for C₁₉H₂₈N₂O₃, 332.21; m/z found, 333.3 [M+H]⁺. ¹H NMR (CDCl₃): 7.83 (d, J=0.8, 1H), 7.33 (d, J=0.8, 1H), 4.61 (br m, 1H), 3.86 (br m, 1H), 3.08 (t, J=7.4, 2H), 2.98 (br m, 1H), 2.51 (br m, 1H), 2.34 (m, 2H), 2.22 (m, 1H), 1.90-1.70 (br m, 6H), 1.58 (br m, 5H), 1.34 (br m, 2H), 1.12 (br m, 4H).

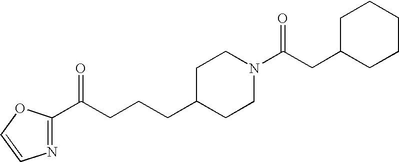

### Example 49

### 4-[1-(2-Cyclohexyl-acetyl)-piperidin-4-yl]-1-oxazol-2-yl-butan-1-one

HPLC: Rₜ=6.9 min. MS (ESI): mass calcd. for C₂₀H₃₀N₂O₃, 346.23; m/z found, 347.4 [M+H]⁺. ¹H NMR (CDCl₃): 7.83 (d, J=0.8, 1H), 7.33 (d, J=0.8, 1H), 4.63 (br m, 1H), 3.87 (br m, 1H), 3.08 (t, J=7.4, 2H), 2.98 (br m, 1H), 2.51 (br m, 1H), 2.20 (d, J=6.9, 2H), 1.84-1.64 (br m, 10H), 1.52 (m, 1H), 1.31 (br m, 4H), 1.11 (br m, 3H), 0.96 (m, 2H).

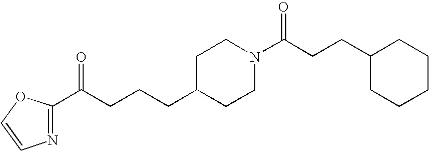

### Example 50

### 4-[1-(3-Cyclohexyl-propionyl)-piperidin-4-yl]-1-oxazol-2-yl-butan-1-one

HPLC: Rₜ=7.4 min. MS (ESI): mass calcd. for C₂₁H₃₂N₂O₃, 360.24; m/z found, 361.4 [M+H]⁺. ¹H NMR (CDCl₃): 7.83 (br s, 1H), 7.33 (br s, 1H), 4.61 (br m, 1H), 3.84 (br m, 1H), 3.08 (t, J=7.4, 2H), 2.98 (br m, 1H), 2.50 (br m, 1H), 2.32 (m, 2H), 1.84-1.60 (br m, 8H), 1.52 (m, 4H), 1.34 (br m, 4H), 1.24-1.00 (br m, 4H), 0.93 (m, 2H).

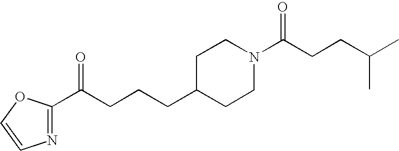

### Example 51

### 4-Methyl-1-[4-(4-oxazol-2-yl-4-oxo-butyl)-piperidin-1-yl]-pentan-1-one

HPLC: Rₜ=4.9 min. MS (ESI): mass calcd. for C₁₈H₂₈N₂O₃, 320.21; m/z found, 321.3 [M+H]⁺. ¹H NMR (CDCl₃): 7.83 (d, J=0.8, 1H), 7.33 (d, J=0.8, 1H), 4.61 (br m, 1H), 3.84 (br m, 1H), 3.08 (t, J=7.4, 2H), 2.98 (br m, 1H), 2.51 (br m, 1H), 2.31 (m, 2H), 1.77 (br m, 4H), 1.64-1.46 (m, 4H), 1.34 (br m, 2H), 1.09 (br m, 2H), 0.91 (d, J=6.4, 6H).

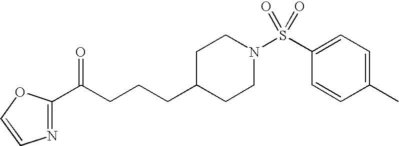

### Example 52

### 1-Oxazol-2-yl-4-[1-(toluene-4-sulfonyl)-piperidin-4-yl]-butan-1-one

To a stirred solution of 1-oxazol-2-yl-4-piperidin-4-yl-butan-1-one hydrochloride (88 mg) and NEt₃ (190 μL) in CH₂Cl₂ (2.0 mL) was added p-toluenesulfonyl chloride (92 mg). After 24 h, the mixture was diluted with CH₂Cl₂ (80 mL) and washed with H₂O (1×20 mL). The organic layer was dried (Na₂SO₄) and concentrated. Chromatographic purification (EtOAc/hexanes) afforded the title compound as a white solid (87 mg, 68%). HPLC: Rₜ=6.7 min. MS (ESI): mass calcd. for C₁₉H₂₄N₂O₃S, 376.15; m/z found, 377.3 [M+H]⁺. ¹H NMR (CDCl₃): 7.81 (d, J=0.4, 1H), 7.64 (m, 2H), 7.32 (m, 2H), 7.26 (d, J=0.4, 1H), 3.76 (br m, 2H), 3.03 (t, J=7.4, 2H), 2.43 (s, 3H), 2.20 (m, 2H), 1.73 (m, 4H), 1.31 (m, 5H).

Examples 53-54 were prepared and purified using methods analogous to those described in Example 52, substituting the appropriate sulfonyl chloride reagents.

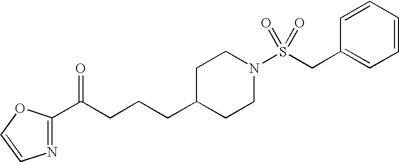

### Example 53

### 1-Oxazol-2-yl-4-(1-phenyl methanesulfonyl-piperidin-4-yl)-butan-1-one

HPLC: Rₜ=6.4 min. MS (ESI): mass calcd. for C₁₉H₂₄N₂O₄S, 376.15; m/z found, 377.3 [M+H]⁺. ¹H NMR (CDCl₃): 7.82 (d, J=0.6, 1H), 7.44-7.33 (br m, 5H), 7.33 (s, 1H), 4.20 (s, 2H), 3.64 (br m, 2H), 3.05 (t, J=7.3, 2H), 2.54 (br m, 2H), 1.74 (br m, 2H), 1.65 (br m, 2H), 1.30 (br m, 3H), 1.14 (br m, 2H).

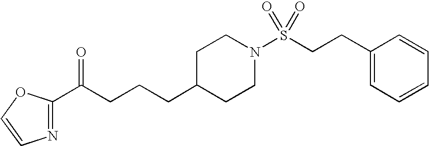

### Example 54

### 1-Oxazol-2-yl-4-[1-(2-phenyl-ethanesulfonyl)-piperidin-4-yl]-butan-1-one

HPLC: Rₜ=6.1 min. MS (ESI): mass calcd. for C₂₀H₂₆N₂O₄S, 390.16; m/z found, 391.3 [M+H]⁺. ¹H NMR (CDCl₃): 7.81 (d, J=0.6, 1H), 7.31 (br m, 6H), 7.20 (m, 2H), 3.79 (m, 2H), 3.06 (t, J=7.3, 2H), 2.70 (br m, 2H), 1.77 (br m, 5H), 1.30 (br m, 6H).

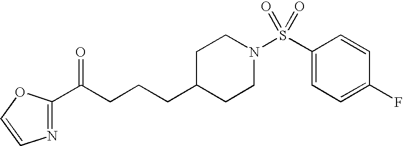

### Example 55

### 4-[1-(4-Fluoro-benzenesulfonyl)-piperidin-4-yl]-1-oxazol-2-yl-butan-1-one

To a mixture of 1-oxazol-2-yl-4-piperidin-4-yl-butan-1-one hydrochloride (100 mg) and satd. aq. NaHCO₃ (0.9 mL) in EtOAc (1.9 mL) was added 4-fluorophenylsulfonyl chloride (225 mg). After 12 h, the mixture was then diluted with EtOAc (30 mL) and washed with H₂O (1×5 mL). The organic layer was dried (Na₂SO₄) and concentrated. Chromatographic purification (EtOAc/hexanes) afforded the title compound as a white solid (120 mg, 41%). HPLC: Rₜ=6.6 min. MS (ESI): mass calcd. for C₁₈H₂₁N₂O₄FS, 380.12; m/z found, 381.2 [M+H]⁺. ¹H NMR (CDCl₃): 7.82 (d, J=0.5, 1H), 7.77 (m, 2H), 7.32 (d, J=0.5, 1H), 7.21 (m, 2H), 3.77 (m, 2H), 3.04 (t, J=7.3, 2H), 2.23 (m, 2H), 1.73 (m, 4H), 1.31 (m, 5H).

Examples 56-68 were prepared and purified using methods analogous to those described in Example 55, substituting the appropriate sulfonyl chloride reagents.

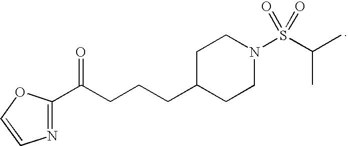

### Example 56

### 1-Oxazol-2-yl-4-[1-(propane-2-sulfonyl)-piperidin-4-yl]-butan-1-one

HPLC: Rₜ=6.0 min. MS (ESI): mass calcd. for C₁₅H₂₄N₂O₄S, 328.15; m/z found, 329.3 [M+H]⁺. ¹H NMR (CDCl₃): 7.83 (d, J=0.4, 1H), 7.33 (d, J=0.4, 1H), 3.80 (br m, 2H), 3.17 (septet, J=6.8, 1H), 3.07 (t, J=7.5, 2H), 2.84 (m, 2H), 1.77 (m, 4H), 1.41-1.21 (m, 5H), 1.33 (d, J=6.8, 6H).

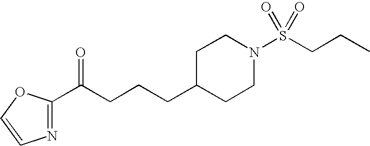

### Example 57

### 1-Oxazol-2-yl-4-[1-(propane-1-sulfonyl)-piperidin-4-yl]-butan-1-one

HPLC: Rₜ=6.1 min. MS (ESI): mass calcd. for C₁₅H₂₄N₂O₄S, 328.15; m/z found, 329.3 [M+H]⁺. ¹H NMR (CDCl₃): 7.83 (d, J=0.7, 1H), 7.33 (d, J=0.7, 1H), 3.79 (br m, 2H), 3.08 (t, J=7.6, 2H), 2.86 (m, 2H), 2.72 (m, 2H), 1.80 (m, 6H), 1.46-1.20 (m, 5H), 1.33 (t, J=6.8, 3H).

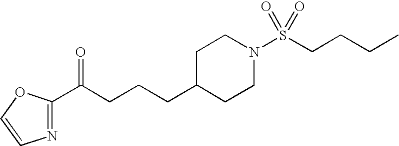

### Example 58

### 4-[1-(Butane-1-sulfonyl)-piperidin-4-yl]-1-oxazol-2-yl-butan-1-one

HPLC: Rₜ=6.4 min. MS (ESI): mass calcd. for C₁₆H₂₆N₂O₄S, 342.16; m/z found, 343.3 [M+H]⁺. ¹H NMR (CDCl₃): 7.83 (d, J=0.7, 1H), 7.33 (d, J=0.7, 1H), 3.79 (br m, 2H), 3.08 (t, J=7.6, 2H), 2.88 (m, 2H), 2.73 (m, 2H), 1.79 (m, 6H), 1.46-1.20 (m, 7H), 0.95 (t, J=7.3, 3H).

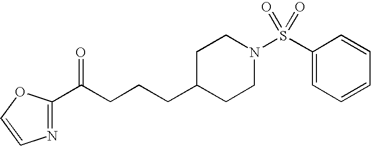

### Example 59

### 4-(1-Benzenesulfonyl-piperidin-4-yl)-1-oxazol-2-yl-butan-1-one

HPLC: Rₜ=6.5 min. MS (ESI): mass calcd. for C₁₈H₂₂N₂O₄S, 362.13; m/z found, 363.3 [M+H]⁺. ¹H NMR (CDCl₃): 7.81 (d, J=0.4, 1H), 7.76 (m, 2H), 7.64-7.50 (m, 3H), 7.31 (d, J=0.4, 1H), 3.79 (br m, 2H), 3.03 (t, J=7.4, 2H), 2.23 (m, 2H), 1.72 (m, 4H), 1.36-1.12 (m, 5H).

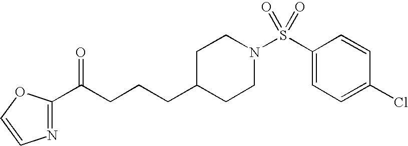

### Example 60

### 4-[1-(4-Chloro-benzenesulfonyl)-piperidin-4-yl]-1-oxazol-2-yl-butan-1-one

HPLC: Rₜ=6.9 min. MS (ESI): mass calcd. for C₁₈H₂₁N₂O₄CIS, 396.09; m/z found, 397.2 [M+H]⁺. ¹H NMR (CDCl₃): 7.82 (d, J=0.8, 1H), 7.70 (m, 2H), 7.51 (m, 2H), 7.32 (d, J=0.8, 1H), 3.76 (br m, 2H), 3.04 (t, J=7.3, 2H), 2.24 (m, 2H), 1.74 (m, 4H), 1.36-1.17 (m, 5H).

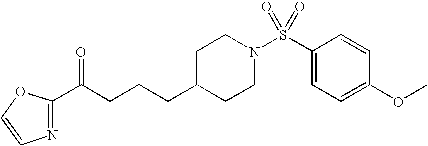

### Example 61

### 4-[1-(4-Methoxy-benzenesulfonyl)-piperidin-4-yl]-1-oxazol-2-yl-butan-1-one

HPLC: Rₜ=6.5 min. MS (ESI): mass calcd. for C₁₉H₂₄N₂O₅S, 392.14; m/z found, 393.3 [M+H]⁺. ¹H NMR (CDCl₃): 7.82 (d, J=0.8, 1H), 7.69 (m, 2H), 7.32 (d, J=0.8, 1H), 7.00 (m, 2H), 3.88 (s, 3H), 3.75 (br m, 2H), 3.03 (t, J=7.3, 2H), 2.21 (m, 2H), 1.72 (m, 4H), 1.36-1.12 (m, 5H).

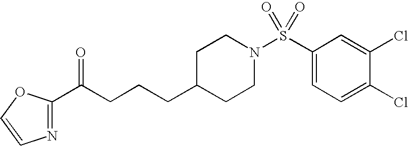

### Example 62

### 4-[1-(3,4-Dichloro-benzenesulfonyl)-piperidin-4-yl]-1-oxazol-2-yl-butan-1-one

HPLC: Rₜ=7.3 min. MS (ESI): mass calcd. for C₁₈H₂₀N₂O₄Cl₂S, 430.05; m/z found, 431.2 [M+H]⁺. ¹H NMR (CDCl₃): 7.84 (d, J=2.1, 1H), 7.82 (s, 1H), 7.60 (m, 2H), 7.32 (s, 1H), 3.78 (br m, 2H), 3.05 (t, J=7.3, 2H), 2.29 (m, 2H), 1.75 (m, 4H), 1.41-1.20 (m, 5H).

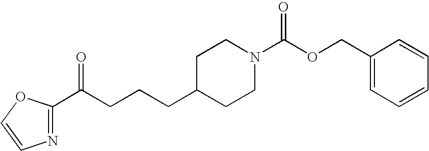

### Example 63

### 4-(4-Oxazol-2-yl-4-oxo-butyl)-piperidine-1-carboxylic acid benzyl ester

To a mixture of 1-oxazol-2-yl-4-piperidin-4-yl-butan-1-one hydrochloride (219 mg) and satd. aq. NaHCO₃ (2.0 mL) in EtOAc (4.0 mL) was added benzyl chloroformate (170 μL). After 2 h, the mixture was diluted with EtOAc (40 mL) and washed with H₂O (1×10 mL). The organic layer was dried (Na₂SO₄) and concentrated. Chromatographic purification (EtOAc/hexanes) afforded the title compound as a white solid (196 mg, 65%). HPLC: Rₜ=6.9 min. MS (ESI): mass calcd. for C₂₀H₂₄N₂O₄, 356.17; m/z found, 357.3 [M+H]⁺. ¹H NMR (CDCl₃): 7.82 (d, J=0.5, 1H), 7.38-7.29 (m, 6H), 5.12 (m, 2H), 4.17 (m, 2H), 3.05 (t, J=7.3, 2H), 2.76 (m, 2H), 1.81-1.61 (m, 4H), 1.46 (m, 1H), 1.33 (m, 2H), 1.12 (m, 2H).

Examples 64-70 were prepared and purified using methods analogous to those described in Example 63, substituting the appropriate chloroformate reagents.

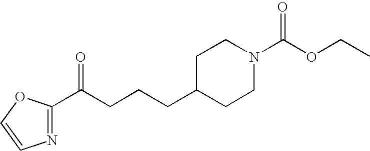

### Example 64

### 4-(4-Oxazol-2-yl-4-oxo-butyl)-piperidine-1-carboxylic acid ethyl ester

HPLC: Rₜ=6.2 min. MS (ESI): mass calcd. for C₁₅H₂₂N₂O₄, 294.16; m/z found, 295.3 [M+H]⁺. ¹H NMR (CDCl₃): 7.82 (d, J=0.6, 1H), 7.33 (d, J=0.6, 1H), 4.12 (m, 4H), 3.07 (t, J=7.5, 2H), 2.72 (m, 2H), 1.78 (m, 2H), 1.69 (m, 2H), 1.43 (m, 1H), 1.33 (m, 2H), 1.25 (t, J=7.0, 3H), 1.10 (m, 2H).

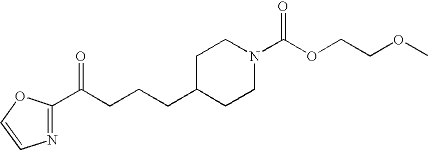

### Example 65

### 4-(4-Oxazol-2-yl-4-oxo-butyl)-piperidine-1-carboxylic acid 2-methoxy-ethyl ester

HPLC: Rₜ=5.7 min. MS (ESI): mass calcd. for C₁₆H₂₄N₂O₅, 324.17; m/z found, 325.4 [M+H]⁺. ¹H NMR (CDCl₃): 7.84 (br s, 1H), 7.33 (br s, 1H), 4.20 (m, 4H), 3.60 (t, J=4.7, 2H), 3.39 (s, 3H), 3.07 (t, J=7.6, 2H), 2.74 (m, 2H), 1.78 (m, 2H), 1.69 (m, 2H), 1.44 (m, 1H), 1.34 (m, 2H), 1.12 (m, 2H).

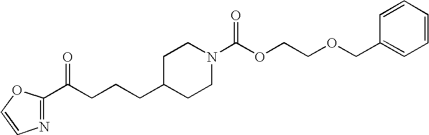

### Example 66

### 4-(4-Oxazol-2-yl-4-oxo-butyl)-piperidine-1-carboxylic acid 2-benzyloxy-ethyl ester

HPLC: Rₜ=6.8 min. MS (ESI): mass calcd. for C₂₂H₂₈N₂O₅, 400.20; m/z found, 401.3 [M+H]⁺. ¹H NMR (CDCl₃): 7.83 (d, J=0.8, 1H), 7.38-7.21 (m, 6H), 4.57 (m, 2H), 4.26 (m, 2H), 4.12 (m, 2H), 3.68 (t, J=4.8, 2H), 3.07 (t, J=7.5, 2H), 2.74 (m, 2H), 1.78 (m, 4H), 1.44 (m, 1H), 1.33 (m, 2H), 1.11 (m, 2H).

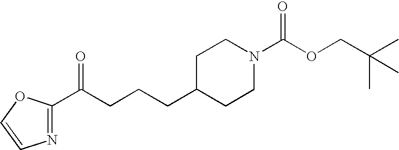

### Example 67

### 4-(4-Oxazol-2-yl-4-oxo-butyl)-piperidine-1-carboxylic acid 2,2-dimethyl-propyl ester

HPLC: Rₜ=7.3 min. MS (ESI): mass calcd. for C₁₈H₂₈N₂O₄, 336.20; m/z found, 337.4 [M+H]⁺. ¹H NMR (CDCl₃): 7.82 (d, J=0.8, 1H), 7.33 (d, J=0.8, 1H), 4.14 (m, 2H), 3.76 (s, 2H), 3.08 (t, J=7.5, 2H), 2.75 (m, 2H), 1.79 (m, 2H), 1.70 (m, 2H), 1.45 (m, 1H), 1.34 (m, 2H), 1.12 (m, 2H), 0.94 (s, 9H).

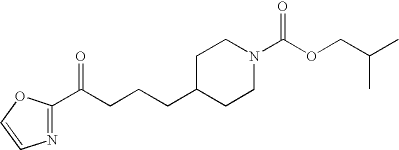

### Example 68

### 4-(4-Oxazol-2-yl-4-oxo-butyl)-piperidine-1-carboxylic acid isobutyl ester

HPLC: Rₜ=7.0 min. MS (ESI): mass calcd. for C₁₇H₂₆N₂O₄, 322.19; m/z found, 323.3 [M+H]⁺. ¹H NMR (CDCl₃): 7.82 (d, J=0.8, 1H), 7.33 (d, J=0.8, 1H), 4.13 (m, 2H), 3.85 (d, J=6.5, 2H), 3.07 (t, J=7.4, 2H), 2.73 (m, 2H), 1.92 (m, 1H), 1.78 (m, 2H), 1.69 (m, 2H), 1.45 (m, 1H), 1.34 (m, 2H), 1.11 (m, 2H), 0.93 (d, J=, 6.9, 6H).

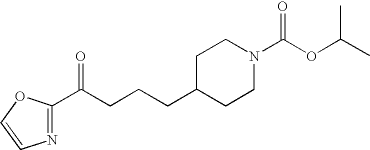

### Example 69

### 4-(4-Oxazol-2-yl-4-oxo-butyl)-piperidine-1-carboxylic acid isopropyl ester

HPLC: Rₜ=6.6 min. MS (ESI): mass calcd. for C₁₆H₂₄N₂O₄, 308.17; m/z found, 309.4 [M+H]⁺. ¹H NMR (CDCl₃): 7.82 (d, J=0.8, 1H), 7.33 (d, J=0.8, 1H), 4.90 (septet, J=6.3, 1H), 4.12 (m, 2H), 3.07 (t, J=7.5, 2H), 2.70 (m, 2H), 1.78 (m, 2H), 1.68 (m, 2H), 1.44 (m, 1H), 1.33 (m, 2H), 1.23 (d, J=6.5, 6H), 0.93 (m, 2H).

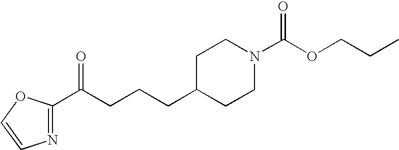

### Example 70

### 4-(4-Oxazol-2-yl-4-oxo-butyl)-piperidine-1-carboxylic acid propyl ester

HPLC: Rₜ=6.7 min. MS (ESI): mass calcd. for C₁₆H₂₄N₂O₄, 308.17; m/z found, 309.3 [M+H]⁺. ¹H NMR (CDCl₃): 7.82 (d, J=0.8, 1H), 7.33 (d, J=0.8, 1H), 4.13 (m, 2H), 4.02 (t, J=6.6, 2H), 3.07 (t, J=7.5, 2H), 2.73 (m, 2H), 1.78 (m, 2H), 1.73-1.60 (br m, 4H), 1.43 (m, 1H), 1.34 (m, 2H), 1.11 (m, 2H), 0.94 (t, J=7.3, 3H).

The compounds in Examples 71-118 were prepared and purified using methods analogous to those described in the preceding examples.

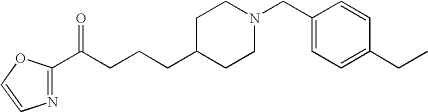

### Example 71

### 4-[1-(4-Ethyl-benzyl)-piperidin-4-yl]-1-oxazol-2-yl-butan-1-one

MS (ESI): mass calcd. for C₂₁H₂₈N₂O₂, 340.22; m/z found, 341.4. ¹H NMR (CDCl₃); 7.81 (s, 1H), 7.45 (d, J=8.3, 2H), 7.30 (s, 1H), 7.22 (d, J=8.3, 2H), 4.07 (s, 2H), 3.36 (br m, 2H), 3.03 (t, J=7.6, 2H), 2.64 (q, J=7.3, 2H), 2.61 (br m, 2H), 1.90-1.80 (br m, 2H), 1.72 (br m, 2H), 1.60 (br m, 1H), 1.44 (br m, 1H), 1.38 (br m, 2H), 1.32 (br m, 1H), 1.22 (t, J=7.3, 3H).

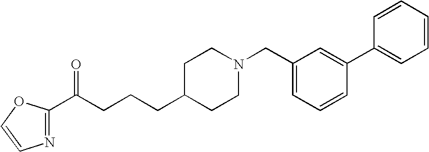

### Example 72

### 4-(1-Biphenyl-3-ylmethyl-piperidin-4-yl)-1-oxazol-2-yl-butan-1-one

MS (ESI): mass calcd. for C₂₅H₂₈N₂O₂, 388.22; m/z found, 389.2. ¹H NMR (CDCl₃): 7.81 (s, 1H), 7.61-7.60 (m, 2H), 7-54 (br s, 1H), 7.49-7.42 (m, 3H), 7.39-7.29 (m, 4H), 3.55 (s, 2H), 3.05 (t, J=7.5, 2H), 2.91 (d, J=10.5, 2H), 1.98-1.94 (m, 2H), 1.76 (heptet, J=7.5, 2H), 1.34-1.27 (br m, 5H).

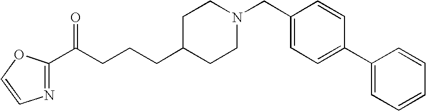

### Example 73

### 4-(1-Biphenyl-4-ylmethyl-piperidin-4-yl)-1-oxazol-2-yl-butan-1-one

MS (ESI): mass calcd. for C₂₅H₂₈N₂O₂, 388.22; m/z found, 389.2. ¹H NMR (CDCl₃): 7.81 (s, 1H), 7.60-7.58 (m, 2H), 7.55-7.53 (m, 2H), 7.44-7.41 (m, 2H), 7.39-7.37 (m, 2H), 7.34-7.32 (m, 2H), 3.52 (s, 2H), 3.06 (t, J=7.0, 2H), 2.90 (d, J=11.0, 2H), 1.98-1.93 (m, 2H), 1.77 (t, J=7.5, 2H), 1.69-1.67 (m, 2H), 1.34-1.24 (br m, 5H).

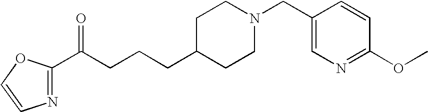

### Example 74

### 4-[1-(6-Methoxy-pyridin-3-ylmethyl)-piperidin-4-yl]-1-oxazol-2-yl-butan-1-one

MS (ESI): mass calcd. for C₁₉H₂₅N₃O₃, 343.19; m/z found, 344.2.

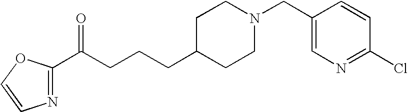

### Example 75

### 4-[1-(6-Chloro-pyridin-3-ylmethyl)-piperidin-4-yl]-1-oxazol-2-yl-butan-1-one

MS (ESI): mass calcd. for C₁₈H₂₂ClN₃O₂, 347.14; m/z found, 348.1.

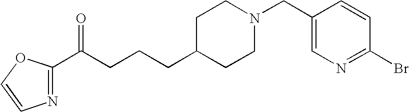

### Example 76

### 4-[1-(6-Bromo-pyridin-3-ylmethyl)-piperidin-4-yl]-1-oxazol-2-yl-butan-1-one

MS (ESI): mass calcd. for C₁₈H₂₂BrN₃O₂, 391.09; m/z found, 392.1.

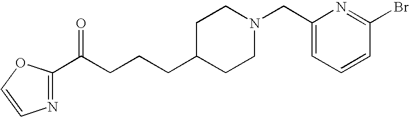

### Example 77

### 4-[1-(6-Bromo-pyridin-2-ylmethyl)-piperidin-4-yl]-1-oxazol-2-yl-butan-1-one

MS (ESI): mass calcd. for C₁₈H₂₂BrN₃O₂, 391.09; m/z found, 392.1.

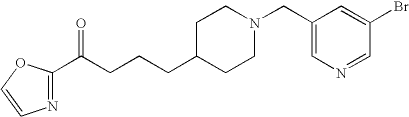

### Example 78

### 4-[1-(5-Bromo-pyridin-3-ylmethyl)-piperidin-4-yl]-1-oxazol-2-yl-butan-1-one

MS (ESI): mass calcd. for C₁₈H₂₂BrN₃O₂, 391.09; m/z found, 392.1.

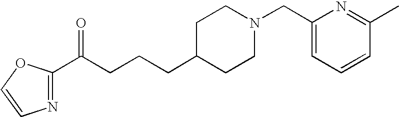

### Example 79

### 4-[1-(6-Methyl-pyridin-2-ylmethyl)-piperidin-4-yl]-1-oxazol-2-yl-butan-1-one

MS (ESI): mass calcd for C₁₉H₂₅N₃O₂, 327.19; m/z found, 328.2.

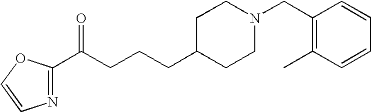

### Example 80

### 4-[1-(2-Methyl-benzyl)-piperidin-4-yl]-1-oxazol-2-yl-butan-1-one

MS (ESI): mass calcd. for C₂₀H₂₆N₂O₂, 326.20; m/z found, 327.2.

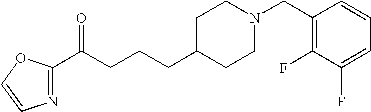

### Example 81

### 4-[1-(2,3-Difluoro-benzyl)-piperidin-4-yl]-1-oxazol-2-yl-butan-1-one

MS (ESI): mass calcd. for C₁₉H₂₂F₂N₂O₂, 348.16; m/z found, 349.2.

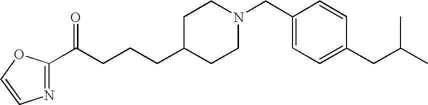

### Example 82

### 4-[1-(4-Isobutyl-benzyl)-piperidin-4-yl]-1-oxazol-2-yl-butan-1-one

MS (ESI): mass calcd. for C₂₃H₃₂N₂O₂, 368.25; m/z found, 369.2. ¹H NMR (CDCl₃): 7.81 (s, 1H), 7.32 (s, 1H), 7.21 (d, J=7.5, 2H), 7.08 (d, J=8.0, 2H), 3.47 (s, 2H), 3.05 (t, J=7.5, 2H), 2.88 (d, J=10.5, 2H), 2.45 (d, J=7.5, 2H), 1.93 (br s, 1H), 1.85 (heptet, J=7.0, 1H), 1.79-1.73 (m, 2H), 1.67-1.65 (m, 2H), 1.33-1.27 (m, 5H), 0.89 (d, J=6.5, 6H).

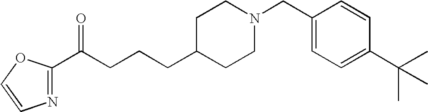

### Example 83

### 4-[1-(4-tert-Butyl-benzyl)-piperidin-4-yl]-1-oxazol-2-yl-butan-1-one

HPLC: Rₜ=4.3 min. MS (ESI): mass calcd. for C₂₃H₃₂N₂O₂, 368.25; m/z found, 369.2 [M+H]⁺. ¹H NMR (CDCl₃): 7.83 (d, J=0.8, 1H), 7.35 (m, 2H), 7.33 (d, J=0.8, 1H), 7.27 (m, 2H), 3.56 (m, 2H), 3.07 (t, J=7.4, 2H), 2.97 (m, 2H), 2.01 (m, 4H), 1.77 (m, 2H), 1.70 (m, 2H), 1.34 (m, 3H), 1.33 (s, 9H).

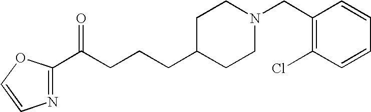

### Example 84

### 4-[1-(2-Chloro-benzyl)-piperidin-4-yl]-1-oxazol-2-yl-butan-1-one

MS (ESI): mass calcd. for C₁₉H₂₃ClN₂O₂, 346.14; m/z found, 347.1.

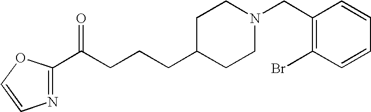

### Example 85

### 4-[1-(2-Bromo-benzyl)-piperidin-4-yl]-1-oxazol-2-yl-butan-1-one

MS (ESI): mass calcd. for C₁₉H₂₃BrN₂O₂, 390.09; m/z found, 391.1.

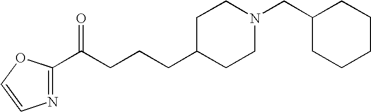

### Example 86

### 4-(1-Cyclohexylmethyl-piperidin-4-yl)-1-oxazol-2-yl-butan-1-one

MS (ESI): mass calcd. for C₁₉H₃₀N₂O₂, 318.23; m/z found, 319.2.

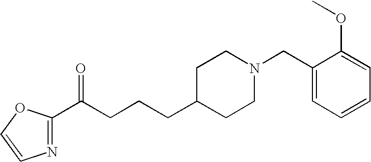

### Example 87

### 4-[1-(2-Methoxy-benzyl)-piperidin-4-yl]-1-oxazol-2-yl-butan-1-one

MS (ESI): mass calcd. for C₂₀H₂₆N₂O₃, 342.19; m/z found, 343.2.

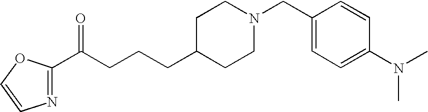

### Example 88

### 4-[1-(4-Dimethylamino-benzyl)-piperidin-4-yl]-1-oxazol-2-yl-butan-1-one

MS (ESI): mass calcd. for C₂₁H₂₉N₃O₂₁ 355.23; m/z found, 223.1.

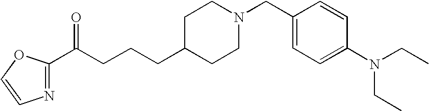

### Example 89

### 4-[1-(4-Diethylamino-benzyl)-piperidin-4-yl]-1-oxazol-2-yl-butan-1-one

MS (ESI): mass calcd. for C₂₃H₃₃N₃O₂, 383.26; m/z found, 223.1.

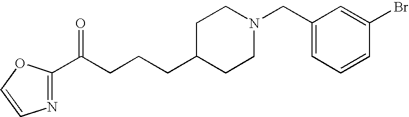

### Example 90

### 4-[1-(3-Bromo-benzyl)-piperidin-4-yl]-1-oxazol-2-yl-butan-1-one

MS (ESI): mass calcd. for C₁₉H₂₃BrN₂O₂, 390.09; m/z found, 391.1.

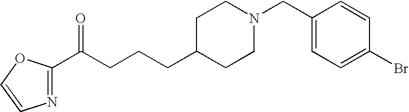

### Example 91

### 4-[1-(4-Bromo-benzyl)-piperidin-4-yl]-1-oxazol-2-yl-butan-1-one

MS (ESI): mass calcd. for C₁₉H₂₃BrN₂O₂, 390.09; m/z found, 392.1.

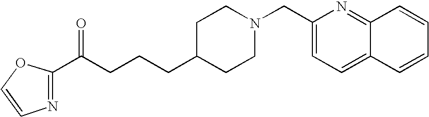

### Example 92

### 1-Oxazol-2-yl-4-(1-quinolin-2-ylmethyl-piperidin-4-yl)-butan-1-one

MS (ESI): mass calcd. for C₂₂H₂₅N₃O₂, 363.19; m/z found, 364.2.

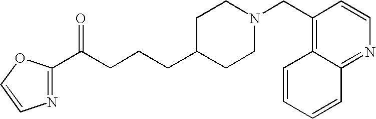

### Example 93

### 1-Oxazol-2-yl-4-(1-quinolin-4-ylmethyl-piperidin-4-yl)-butan-1-one

MS (ESI): mass calcd. for C₂₂H₂₅N₃O₂, 363.19; m/z found, 364.2.

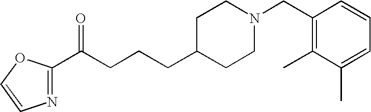

### Example 94

### 4-[1-(2,3-Dimethyl-benzyl)-piperidin-4-yl]-1-oxazol-2-yl-butan-1-one

MS (ESI): mass calcd. for C₂₁H₂₈N₂O₂, 340.22; m/z found, 341.2.

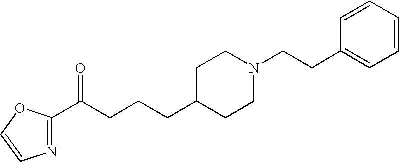

### Example 95

### 1-Oxazol-2-yl-4-(1-phenethyl-piperidin-4-yl)-butan-1-one

MS (ESI): mass calcd. for C₂₀H₂₆N₂O₂, 326.20; m/z found, 327.2.

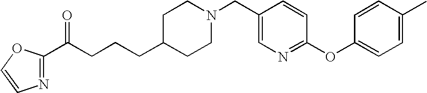

### Example 96

### 1-Oxazol-2-yl-4-[1-(6-p-tolyloxy-pyridin-3-ylmethyl)-piperidin-4-yl]-butan-1-one

MS (ESI): mass calcd. for C₂₅H₂₉N₃O₃, 419.22; m/z found, 420.2.

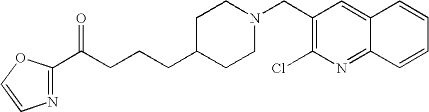

### Example 97

### 4-[1-(2-Chloro-quinolin-3-ylmethyl)-piperidin-4-yl]-1-oxazol-2-yl-butan-1-one

HPLC: Rₜ=3.9 min. MS (ESI): mass calcd. for C₂₃H₂₅ClN₂O₂, 396.91; m/z found, 398.1 [M+H]⁺. ¹H NMR (CDCl₃): 8.26 (s, 1H), 8.02 (d, J=8.5, 1H), 7.85 (m, 2H), 7.70 (m, 1H), 7.56 (m, 1H), 7.34 (s, 1H), 3.72 (s, 2H), 3.09 (t, J=7.4, 2H), 2.96 (m, 2H), 2.17 (m, 2H), 1.81 (m, 2H), 1.74 (br m, 2H), 1.43-1.34 (m, 6H).

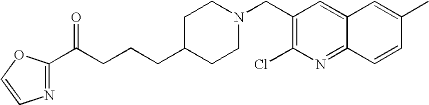

### Example 98

### 4-[1-(2-Chloro-6-methyl-quinolin-3-ylmethyl)-piperidin-4-yl]-1-oxazol-2-yl-butan-1-one

HPLC: Rₜ=3.9 min. MS (ESI): mass calcd. for C₂₄H₂₇ClN₂O₂, 410.94; m/z found, 412.2 [M+H]⁺. ¹H NMR (CDCl₃): 8.18 (s, 1H), 7.90 (d, J=8.5, 1H), 7.83 (s, 1H), 7.61 (s, 1H), 7.53 (m, 1H), 7.34 (s, 1H), 3.71 (s, 2H), 3.09 (t, J=7.5, 2H), 2.96 (m, 2H), 2.54 (s, 3H), 2.17 (m, 2H), 1.81 (m, 2H), 1.73 (br m, 2H), 1.43-1.34 (m, 6H).

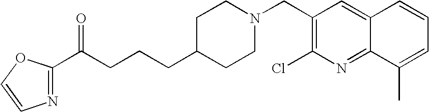

### Example 99

### 4-[1-(2-Chloro-8-methyl-quinolin-3-ylmethyl)-piperidin-4-yl]-1-oxazol-2-yl-butan-1-one

HPLC: Rₜ=4.1 min. MS (ESI): mass calcd. for C₂₄H₂₇ClN₂O₂, 410.94; m/z found, 412.2 [M+H]⁺. ¹H NMR (CDCl₃): 8.21 (s, 1H), 7.84 (s, 1H), 7.67 (d, J=6.9, 1H), 7.54 (d, J=7.0, 1H), 7.44 (m, 1H), 7.34 (s, 1H), 3.72 (s, 2H), 3.09 (t, J=7.5, 2H), 2.96 (m, 2H), 2.78 (s, 3H), 2.17 (m, 2H), 1.81 (m, 2H), 1.73 (br m, 2H), 1.36 (m, 6H).

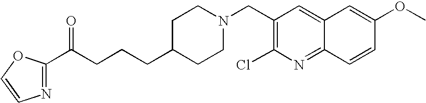

### Example 100

### 4-[1-(2-Chloro-6-methoxy-quinolin-3-ylmethyl)-piperidin-4-yl]-1-oxazol-2-yl-butan-1-one

MS (ESI): mass calcd. for C₂₃H₂₆ClN₃O₃, 427.17; m/z found, 428.1. ¹H NMR (CDCl₃): 8.16 (s, 1H), 7.89 (d, J=9.0, 1H), 7.82 (s, 1H), 7.35-7.32 (m, 2H), 7.09 (d, J=3.0, 1H), 3.93 (s, 3H), 3.70 (s, 2H), 3.08 (t, J=7.5, 2H), 2.95 (d, J=11.0, 2H), 2.18-2.14 (m, 2H), 1.83-1.72 (m, 5H), 1.38-1.35 (m, 4H).

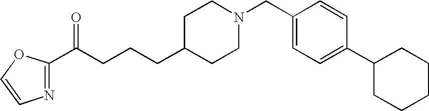

### Example 101

### 4-[1-(4-Cyclohexyl-benzyl)-piperidin-4-yl]-1-oxazol-2-yl-butan-1-one

MS (ESI): mass calcd. for C₂₅H₃₄N₂O₂, 394.26; m/z found, 395.2. ¹H NMR (CDCl₃): 7.81 (d, J=0.8, 1H), 7.41 (d, J=8.1, 2H), 7.30 (s, 1H), 7.22 (d, J=8.1, 2H), 3.98 (s, 2H), 3.31 (br m, 2H), 3.03 (br t, J=7.1, 2H), 2.49 (br m, 2H), 1.82 (br m, 8H), 1.73 (br m, 4H), 1.39 (br m, 8H).

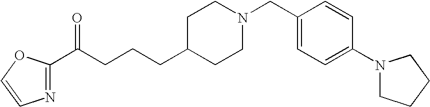

### Example 102

### 1-oxazol-2-yl-4-[1-(4-pyrrolidin-1-yl-benzyl)-piperidin-4-yl]-butan-1-one

MS (ESI): mass calcd. for C₂₃H₃₁N₃O₂, 381.24; m/z found, 223.1.

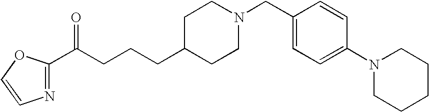

### Example 103

### 1-Oxazol-2-yl-4-[1-(4-piperidin-1-yl-benzyl)-piperidin-4-yl]-butan-1-one

MS (ESI): mass calcd. for C₂₄H₃₃N₃O₂, 395.26; m/z found, 223.2.

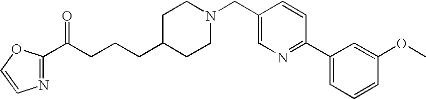

### Example 104

### 4-{1-[6-(3-Methoxy-phenyl)-pyridin-3-ylmethyl]-piperidin-4-yl}-1-oxazol-2-yl-butan-1-one

MS (ESI): mass calcd. for C₂₅H₂₉N₃O₃, 419.22; m/z found, 420.2. ¹H NMR (CDCl₃): 8.58 (m, 1H), 7.81 (s, 1H), 7.75-7.73 (m, 1H), 7.69-7.67 (m, 1H), 7.59-7.58 (m, 1H), 7.55-7.53 (m, 1H), 7.37 (t, J=8.0, 1H), 7.32 (s, 1H), 6.97-6.95 (m, 1H), 3.90 (s, 3H), 3.53 (s, 2H), 3.06 (t, J=7.5, 2H), 2.89 (d, J=11.5, 2H), 1.99 (t, J=10.5, 2H), 1.80-1.74 (m, 2H), 1.70 (d, J=10.5, 2H), 1.35-1.27 (m, 5H).

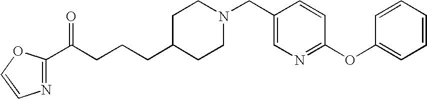

### Example 105

### 1-Oxazol-2-yl-4-[1-(6-phenoxy-pyridin-3-ylmethyl)-piperidin-4-yl]-butan-1-one

MS (ESI): mass calcd. for C₂₄H₂₇N₃O₃, 405.21; m/z found, 406.2. ¹H NMR (CDCl₃): 8.07 (d, J=2.0, 1H), 7.81 (s, 1H), 7.68 (d, J=7.5, 1H), 7.41-7.38 (m, 2H), 7.32 (s, 1H), 7.20-7.18 (m, 1H), 7.14-7.12 (m, 2H), 6.86 (d, J=8.0, 1H), 3.44 (s, 2H), 3.06 (t, J=7.5, 2H), 2.85 (d, J=11.0, 2H), 1.94 (t, J=10.0, 2H), 1.79-1.73 (m, 2H), 1.67 (d, J=11.0, 2H), 1.34-1.20 (m, 5H).

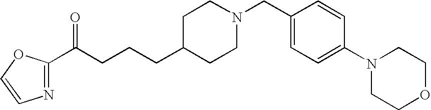

### Example 106

### 4-[1-(4-Morpholin-4-yl-benzyl)-piperidin-4-yl]-1-oxazol-2-yl-butan-1-one

MS (ESI): mass calcd. for C₂₃H₃₁N₃O₃, 397.24; m/z found, 398.2.

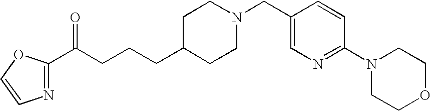

### Example 107

### 4-[1-(6-Morpholin-4-yl-pyridin-3-ylmethyl)-piperidin-4-yl]-1-oxazol-2-yl-butan-1-one

MS (ESI): mass calcd. for C₂₂H₃₀N₄O₃, 398.23; m/z found, 399.2.

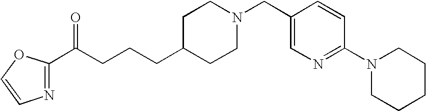

### Example 108

### 1-oxazol-2-yl-4-[1-(3,4,5,6-tetrahydro-2H-[1,2′]bipyridinyl-5′-ylmethyl)-piperidin-4-yl]-butan-1-one

MS (ESI): mass calcd. for C₂₃H₃₂N₄O₂, 396.25; m/z found, 397.2.

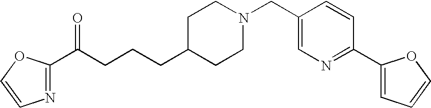

### Example 109

### 4-[1-(6-Furan-2-yl-pyridin-3-ylmethyl)-piperidin-4-yl]-1-oxazol-2-yl-butan-1-one

MS (ESI): mass calcd. for C₂₂H₂₅N₃O₃, 379.19; m/z found, 380.2.

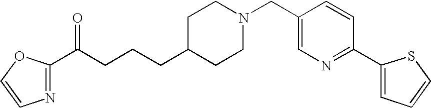

### Example 110

### 1-Oxazol-2-yl-4-[1-(6-thiophen-2-yl-pyridin-3-ylmethyl)-piperidin-4-yl]-butan-1-one

MS (ESI): mass calcd. for C₂₂H₂₅N₃O₂S, 395.17; m/z found, 396.2.

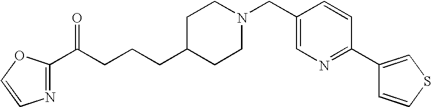

### Example 111

### 1-Oxazol-2-yl-4-[1-(6-thiophen-3-yl-pyridin-3-ylmethyl)-piperidin-4-yl]-butan-1-one

MS (ESI): mass calcd. for C₂₂H₂₅N₃O₂S, 395.17; m/z found, 396.1.

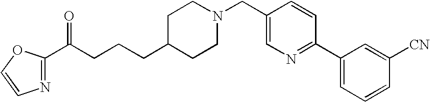

### Example 112

### 3-{5-[4-(4-Oxazol-2-yl-4-oxo-butyl)-piperidin-1-ylmethyl]-pyridin-2-yl}-benzonitrile

MS (ESI): mass calcd. for C₂₅H₂₆N₄O₂, 414.21; m/z found, 415.2.

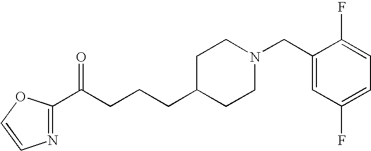

### Example 113

### 4-[1-(2,5-Difluoro-benzyl)-piperidin-4-yl]-1-oxazol-2-yl-butan-1-one

MS (ESI): mass calcd. for C₁₉H₂₂F₂N₂O₂, 348.16; m/z found, 349.1.

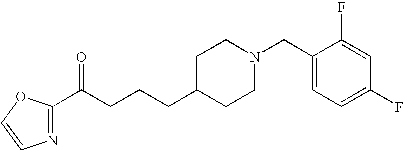

### Example 114

### 4-[1-(2,4-Difluoro-benzyl)-piperidin-4-yl]-1-oxazol-2-yl-butan-1-one

MS (ESI): mass calcd. for C₁₉H₂₂F₂N₂O₂, 348.16; m/z found, 349.1.

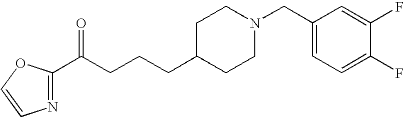

### Example 115

### 4-[1-(3,4-Difluoro-benzyl)-piperidin-4-yl]-1-oxazol-2-yl-butan-1-one

MS (ESI): mass calcd. for C₁₉H₂₂F₂N₂O₂, 348.16; m/z found, 349.2.

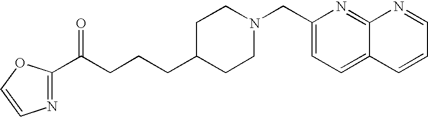

### Example 116

### 4-(1-[1,8]Naphthyridin-2-ylmethyl-piperidin-4-yl)-1-oxazol-2-yl-butan-1-one

MS (ESI): mass calcd. for C₂₁H₂₄N₄O₂, 364.19; m/z found, 365.2.

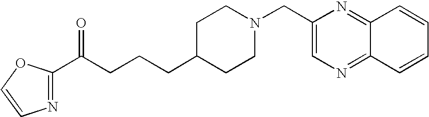

### Example 117

### 1-Oxazol-2-yl-4-(1-quinoxalin-2-ylmethyl-piperidin-4-yl)-butan-1-one

MS (ESI): mass calcd. for C₂₁H₂₄N₄O₂, 364.19; m/z found, 365.2.

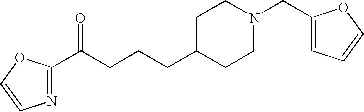

### Example 118

### 4-(1-Furan-2-ylmethyl-piperidin-4-yl)-1-oxazol-2-yl-butan-1-one

MS (ESI): mass calcd. for C₁₇H₂₂N₂O₃, 302.16; m/z found, 303.1.

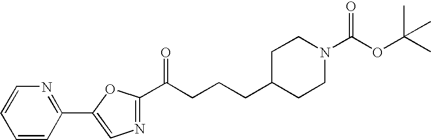

### Example 119

### 4-[4-Oxo-4-(5-pyridin-2-yl-oxazol-2-yl)-butyl]-piperidine-1-carboxylic acid tert-butyl ester

The title compound was prepared using methods similar to those described in Example 1 (alternative method), with the following alterations: 2-oxazol-5-yl-pyridine (Saikachi et al. Chem. Pharm Bull. 1979, 27, 793-796) was substituted for oxazole; the reaction between the oxazole Grignard and the Weinreb amide was executed at 66° C. over 3 days; careful column chromatography was needed to isolate the pure product in a yield of 17%. MS (ESI): mass calcd. for C₂₀H₂₃NO₄, 399.22; m/z found, 400.0 [M+H]⁺. ¹H NMR (CDCl₃): 8.68-8.66 (m, 1H), 7.88 (s, 1H), 7.89-7.86 (m, 1H), 7.82 (td, J=7.7, 1.8, 1H), 7.35-7.29 (m, 1H), 4.08 (br s, 2H), 3.11 (t, J=7.4, 2H), 2.68 (br t, J=12.0, 2H), 1.86-1.77 (m, 2H), 1.68 (br d, J=12.9, 2H), 1.45 (s, 9H), 1.48-1.38 (m, 1H), 1.39-1.31 (m, 2H), 1.16-1.03 (m, 2H).

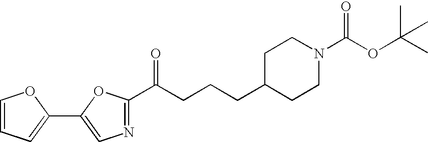

### Example 120

### 4-[4-(5-Furan-2-yl-oxazol-2-yl)-4-oxo-butyl]-Piperidine-1-carboxylic acid tert-butyl ester

To a stirred solution of 5-furan-2-yl-oxazole (140 mg) in THF (8.0 mL) at −78° C. was added nBuLi (1.6 M in hexanes, 0.71 mL), and the resulting solution was stirred for 30 min at −78° C. ZnCl₂ (1.0 M in Et₂₀, 1.1 mL) was added, and the mixture was stirred for 30 min at −78° C. before warming to 0° C. After 15 min, CuI (217 mg) was added, and the resulting suspension was stirred for 30 min at 0° C. Next, a solution of 4-(3-chlorocarbonyl-propyl)-piperidine-1-carboxylic acid tert-butyl ester (330 mg) in THF (2.0 mL) was added via cannula, and the resulting mixture was stirred at 0° C. for 1 h. The mixture was diluted with EtOAc (200 mL) and washed with H₂O (1×50 mL). The organic layer was dried (Na₂SO₄) and concentrated. Chromatographic purification (EtOAc/hexanes) afforded the title compound as a pale yellow oil (84 mg, 21%). HPLC: Rₜ=7.7 min. MS (ESI): mass calcd. for C₂₁H₂₈N₂O₅, 388.4; m/z found, 411.4 [M+Na]⁺. ¹H NMR (CDCl₃): 7.54 (dd, J=1.8, 0.6 Hz, 1H), 7.41 (br s, 1H), 6.90 (br d, J=3.5 Hz, 1H), 6.55 (dd, J=3.5, 2.0 Hz, 1H), 4.07 (br m, 2H), 3.07 (t, J=7.3, 2H), 2.67 (br m, 2H), 1.79 (m, 2H), 1.67 (m, 2H), 1.45 (s, 9H), 1.39-1.28 (m, 3H), 1.09 (m, 2H).

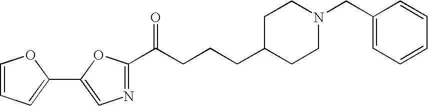

### Example 121

### 4-(1-Benzyl-piperidin-4-yl)-1-(5-furan-2-yl-oxazol-2-yl)-butan-1-one

Step A. 1-(5-Furan-2-yl-oxazol-2-yl)-4-piperidin-4-yl-butan-1-one trifluoroacetate. To a solution of 4-[4-(5-furan-2-yl-oxazol-2-yl)-4-oxo-butyl]-piperidine-1-carboxylic acid tert-butyl ester (112 mg) in CH₂Cl₂ (3.0 mL) was added TFA (0.45 mL). After 30 min at rt, the mixture was concentrated to afford the title compound as a brown oil (114 mg, 98%). HPLC: Rₜ=4.3 min. MS (ESI): mass calcd. for C₁₆H₂₀N₂O₃, 288.34; m/z found, 289.3 [M+H]⁺. ¹H NMR (CD₃OD): 7.55 (d, J=1.7, 1H), 7.37 (s, 1H), 6.80 (d, J=3.5 Hz, 1H), 6.48 (dd, J=3.5, 1.8 Hz, 1H), 3.23 (br m, 2H), 2.93 (t, J=7.3, 2H), 2.82 (br m, 2H), 1.82 (m, 2H), 1.63 (m, 2H), 1.49 (m, 2H), 1.30-1.15 (m, 4H).

Step, B. To a stirred solution of 1-(5-furan-2-yl-oxazol-2-yl)-4-piperidin-4-yl-butan-1-one trifluoroacetate (58 mg) and benzaldehyde (16 μL) in CH₂Cl₂ (1.0 mL) was added NaB(OAc)₃H (46 mg). After 24 h, the mixture was filtered through a short pad of SiO₂ (MeOH/CH₂Cl₂) and the filtrate was concentrated. Chromatographic purification (MeOH/CH₂Cl₂) afforded the title compound as a pale yellow oil (20 mg, 38%). HPLC: Rₜ=4.8 min. MS (ESI): mass calcd. for C₂₃H₂₆N₂O₃, 378.46; m/z found, 379.3 [M+H]⁺. ¹H NMR (CDCl₃): 7.54 (dd, J=2.0, 0.6, 1H), 7.41-7.29 (m, 6H), 6.89 (d, J=3.6, 1H), 6.55 (dd, J=3.6, 2.0, 1H), 3.66 (br m, 4H), 3.05 (br m, 4H), 2.14 (m, 2H), 1.80-1.60 (m, 3H), 1.55-1.21 (m, 4H).

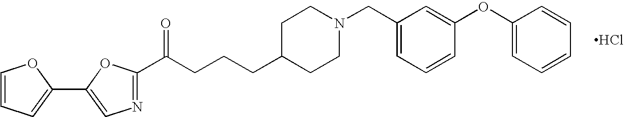

### Example 122

### 1-Oxazol-2-yl-4-[1-(3-phenoxy-benzyl)-piperidin-4-yl]-butan-1-one hydrochloride

A solution of 1-oxazol-2-yl-4-[1-(3-phenoxy-benzyl)-piperidin-4-yl]-butan-1-one (390 mg) and HCl (2.0 M in Et₂O, 8.0 mL) was stirred at rt for 4 h. The mixture was concentrated, affording the title compound as pale yellow solid (371 mg, 87%). HPLC: Rₜ=4.9 min. MS (ESI): mass calcd. for C₂₅H₂₈N₂O₃, 404.21; m/z found, 405.3 [M+H]⁺. ¹H NMR (CD₃OD): 8.11 (d, J=0.8, 1H), 7.48-7.36 (m, 4H), 7.29 (m, 1H), 7.21 (m, 1H), 7.16 (m, 1H), 7.07 (m, 1H), 7.03 (m, 2H), 4.29 (br s, 2H), 3.47 (br m, 2H), 3.07 (t, J=7.0, 2H), 2.98 (m, 2H), 1.97 (m, 4H), 1.75 (m, 2H), 1.62 (m, 1H), 1.47 (m, 2H).

The compounds in Examples 123-129 were prepared using methods analogous to those described in the preceding examples.

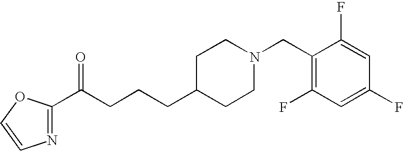

### Example 123

### 1-Oxazol-2-yl-4-[1-(2,4,6-trifluoro-benzyl)-piperidin-4-yl]-butan-1-one

MS (ESI): mass calcd. for C₁₉H₂₁F₃N₂O₂, 366.16; m/z found, 367.1.

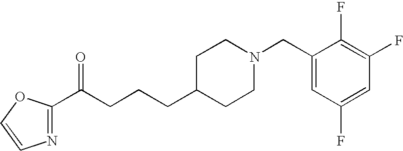

### Example 124

### 1-Oxazol-2-yl-4-[1-(2,3,5-trifluoro-benzyl)-piperidin-4-yl]-butan-1-one

MS (ESI): mass calcd. for C₁₉H₂₁F₃N₂O₂, 366.16; m/z found, 367.1.

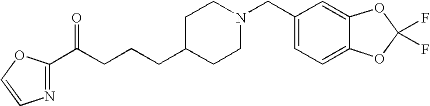

### Example 125

### 4-[1-(2,2-Difluoro-benzo[1,3]dioxol-5-ylmethyl)-piperidin-4-yl]-1-oxazol-2-yl-butan-1-one

MS (ESI): mass calcd. for C₂₀H₂₂F₂N₂O₄, 392.15; m/z found, 393.1. ¹H NMR (CDCl₃): 7.82 (m, 1H), 7.33 (m, 1H), 7.09 (s, 1H), 6.98-6.97 (m, 2H), 3.44 (s, 2H), 3.06 (t, J=7.6, 2H), 2.82 (d, J=11.6, 2H), 1.93 (t, J=11.2, 2H), 1.80-1.73 (m, 2H), 1.67 (d, J=10.8, 2H), 1.35-1.21 (m, 5H).

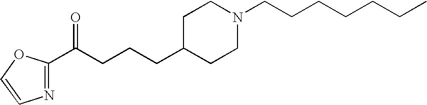

### Example 126

### 4-(1-Heptyl-piperidin-4-yl)-1-oxazol-2-yl-butan-1-one

MS (ESI): mass calcd. for C₁₉H₃₂N₂O₂, 320.25; m/z found, 321.2.

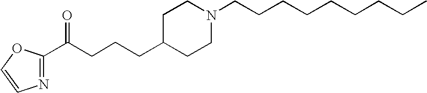

### Example 127

### 4-(1-Nonyl-Piperidin-4-yl)-1-oxazol-2-yl-butan-1-one

MS (ESI): mass calcd. for C₂₁H₃₆N₂O₂, 348.28; m/z found, 349.2. ¹H NMR (CDCl₃): 7.82 (m, 1H), 7.33 (m, 1H), 3.06 (t, J=7.6, 2H), 2.93 (d, J=11.2, 2H), 2.32-2.28 (m, 2H), 1.89 (t, J=10.4, 2H), 1.81-1.73 (m, 2H), 1.71-1.68 (m, 2H), 1.53-1.48 (br m, 2H), 1.34-1.26 (br m, 17H), 0.88 (t, J=6.8, 3H).

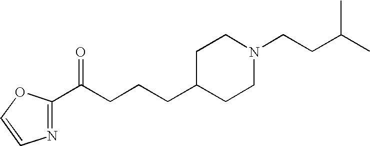

### Example 128

### 4-[1-(3-Methyl-butyl)-piperidin-4-yl]-1-oxazol-2-yl-butan-1-one

MS (ESI): mass calcd. for C₁₇H₂₈N₂O₂, 292.22; m/z found, 293.2.

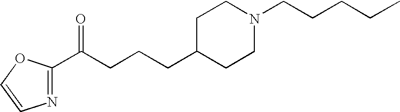

### Example 129

### 1-Oxazol-2-yl-4-(1-pentyl-piperidin-4-yl)-butan-1-one

MS (ESI): mass calcd. for C₁₇H₂₈N₂O₂, 292-22; m/z found, 293.2.

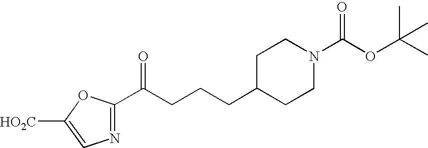

### Example 130

### 4-[4-(5-Carboxy-oxazol-2-yl)-4-oxo-butyl]-piperidine-1-carboxylic acid tert-butyl ester

Step A. 4-(4-Hydroxy-4-oxazol-2-yl-butyl)-piperidine-1-carboxylic acid tert-butyl ester. To a stirred solution of 4-(4-oxazol-2-yl-4-oxo-butyl)-piperidine-1-carboxylic acid tert-butyl ester (10.3 g) in MeOH (200 mL) was added NaBH₄ (1.82 g). The resulting solution was stirred at rt for 4 h and then concentrated. The crude residue was then partitioned between CH₂Cl₂ (200 mL) and satd. aq. NaHCO₃ (20 mL). The organic layer was separated, dried (Na₂SO₄), filtered and concentrated. Purification by silica gel flash chromatography (EtOAc) afforded the title compound as colorless oil (10.1 g, 97%). HPLC: Rₜ=6.1 min. MS (ESI): mass calcd. for C₁₇H₂₈N₂O₄, 324.20; m/z found, 347.1 [M+Na]⁺. ¹H NMR (CDCl₃): 7.63 (d, J=1.1, 1H), 7.07 (d, J=1.1, 1H), 4.81 (br m, 1H), 4.06 (br m, 2H), 3.45 (d, J=5.5, 1H), 2.65 (br m, 2H), 1.89 (m, 2H), 1.63 (m, 2H), 1.45 (s, 9H), 1.38 (m, 2H), 1.27 (m, 3H), 1.05 (m, 2H).

Step B. 4-[4-(tert-Butyl-dimethyl-silanyloxy)-4-oxazol-2-yl-butyl]-piperidine-1-carboxylic acid tert-butyl ester. To a stirred solution of 4-(4-hydroxy-4-oxazol-2-yl-butyl)-piperidine-1-carboxylic acid tert-butyl ester (4.50 g) and imidazole (2.83 g) in CH₂Cl₂ (100 mL) was added tert-butyldimethylsilyl chloride (2.51 g). The resulting solution was stirred at rt for 24 h and then concentrated. The crude residue was purified by silica gel flash chromatography (EtOAc/hexanes) to give the title compound as a colorless oil (6.01 g, 99%). HPLC: Rₜ=10.0 min. MS (ESI): mass calcd. for C₂₃H₄₂N₂O₄Si, 438.29; m/z found, 461.2 [M+Na]⁺. ¹H NMR (CDCl₃): 7.60 (d, J=1.1, 1H), 7.05 (d, J=1.1, 1H), 4.80 (dd, J=7.6, 5.8, 1H), 4.06 (br m, 2H), 2.64 (br m, 2H), 1.84 (m, 2H), 1.61 (m, 2H), 1.45 (s, 9H), 1.37 (m, 2H), 1.25 (m, 3H), 1.04 (m, 2H), 0.86 (s, 9H), 0.06 (s, 3H), −0.06 (s, 3H).

Step C. 4-[4-(tert-Butyl-dimethyl-silanyloxy)-4-(5-carboxy-oxazol-2-yl)-butyl]-piperidine-1-carboxylic acid tert-butyl ester. To a stirred solution of 4-[4-(tert-butyl-dimethyl-silanyloxy)-4-oxazol-2-yl-butyl]-piperidine-1-carboxylic acid tert-butyl ester (3.25 g) in THF (80 mL) at −78° C. was added tert-butyllithium (4.80 mL, 1.7 M in pentane). The resulting solution was stirred at −78° C. for 30 min followed by the addition of solid carbon dioxide (1.30 g). After 30 min the reaction was vented and slowly warmed to rt. Concentration of the colorless solution afforded the title compound as a white solid (3.55 g, 99%). HPLC: Rₜ=8.7 min. MS (ESI): mass calcd. for C₂₄H₄₂N₂O₆Si, 482.28; m/z found, 481.2 [M−H]⁺. ¹H NMR (CDCl₃): 7.58 (br s, 1H), 4.76 (br t, J=6.6, 1H), 4.01 (br m, 2H), 2.88 (br m, 2H), 2.58 (br m, 2H), 1.81 (m, 2H), 1.53 (m, 2H), 1.44 (s, 9H), 1.31 (m, 2H), 1.17 (m, 2H), 0.96 (m, 2H), 0.84 (s, 9H), 0.04 (s, 3H), −0.05 (s, 3H).

Step D. 4-[4-(5-Carboxy-oxazol-2-yl)-4-hydroxy-butyl]-piperidine-1-carboxylic acid tert-butyl ester. To a stirred solution of 4-[4-(tert-butyl-dimethyl-silanyloxy)-4-(5-carboxy-oxazol-2-yl)-butyl]-piperidine-1-carboxylic acid tert-butyl ester (250 mg) in THF (5.0 mL) at rt was added tetrabutylammonium fluoride (1.60 mL, 1.0 M in THF). The resulting solution was stirred at rt for 1 h and then concentrated. Purification by reverse-phase HPLC afforded the title compound as a colorless oil (170 mg, 89%). HPLC: Rₜ=5.5 min. MS (ESI): mass calcd. for C₁₈H₂₈N₂O₆, 368.19; m/z found, 367.2 [M−H]⁺. ¹H NMR (CDCl₃): 7.77 (br s, 1H), 5.32 (br s, 1H), 4.88 (br t, J=6.6, 1H), 4.04 (br m, 2H), 3.49 (s, 2H), 2.65 (br m, 2H), 1.92 (br m, 2H), 1.62 (m, 2H), 1.44 (s, 9H), 1.37 (m, 2H), 1.27 (m, 2H), 1.05 (m, 2H).

Step E. To a stirred solution of 4-[4-(5-carboxy-oxazol-2-yl)-4-hydroxy-butyl]-piperidine-1-carboxylic acid tert-butyl ester (40 mg) in CH₂Cl₂ (1.0 mL) at rt was added the Dess-Martin periodinane (64 mg). The resulting solution was stirred at rt for 16 h and then diluted with MeOH and concentrated. Purification by reverse-phase HPLC afforded the title compound as a colorless oil (14.9 mg, 37%). HPLC: Rₜ=6.4 min. MS (ESI): mass calcd. for C₁₈H₂₆N₂O₆, 366.18; m/z found, 365.2 [M−H]⁺. ¹H NMR (CDCl₃): 7.93 (br s, 1H), 4.33 (br s, 1H), 4.08 (br m, 2H), 3.09 (t, J=7.4, 2H), 2.70 (br m, 2H), 1.78 (br m, 2H), 1.68 (m, 2H), 1.46 (s, 9H), 1.44 (m, 1H), 1.33 (m, 2H), 1.10 (m, 2H).

Examples 131-140 were prepared and purified using methods analogous to those described in preceding examples.

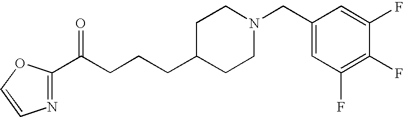

### Example 131

### 1-Oxazol-2-yl-4-[1-(3,4,5-trifluoro-benzyl)-piperidin-4-yl]-butan-1-one

HPLC: Rₜ=4.0 min. MS (ESI): mass calcd. for C₁₉H₂₁F₃N₂O₂, 366.16; m/z found, 367.1 [M+H]⁺. ¹H NMR (CDCl₃): 7.82 (s, 1H), 7.33 (s, 1H), 6.96 (t, J=7.0, 2H), 3.39 (s, 2H), 3.06 (t, J=7.5, 2H), 2.80 (d, J=12.0, 2H), 1.95 (t, J=11.0, 2H), 1.80-1.74 (m, J=7.5, 2H), 1.67 (d, J=11.0, 2H), 1.35-1.21 (br m, 5H).

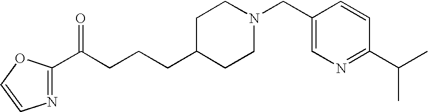

### Example 132

### 4-[1-(6-Isopropyl-pyridin-3-ylmethyl)-piperidin-4-yl]-1-oxazol-2-yl-butan-1-one

HPLC: Rₜ=3.5 min. MS (ESI): mass calcd. for C₂₁H₂₉N₃O₂, 355.23; m/z found, 356.2 [M+H]⁺. ¹H NMR (CDCl₃): 8.41 (m, 1H), 7.81 (s, 1H), 7.59 (d, J=7.0, 1H), 7.32 (s, 1H), 7.13 (d, J=8.0, 1H), 3.45 (s, 2H), 3.07-3.04 (br m, 3H), 2.86 (d, J=11.5, 2H), 1.94 (t, J=11.0, 2H), 1.79-1.73 (m, 2H), 1.67 (d, J=11.0, 2H), 1.30 (d, J=6.5, 6H), 1.34-1.22 (br m, 5H).

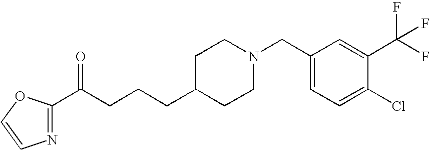

### Example 133

### 4-[1-(4-Chloro-3-trifluoromethyl-benzyl)-piperidin-4-yl]-1-oxazol-2-yl-butan-1-one

HPLC: Rₜ=4.2 min. MS (ESI): mass calcd. for C₂₀H₂₂ClF₃N₂O₂, 414.13; m/z found, 415.1 [M+H]⁺. ¹H NMR (CDCl₃): 7.82 (s, 1H), 7.64 (s, 1H), 7.43 (br s, 2H), 7.33 (s, 1H), 3.47 (s, 2H), 3.06 (t, J=7.5, 2H), 2.81 (d, J=11.5, 2H), 1.95 (t, J=11.5, 2H), 1.80-1.74 (m, 2H), 1.67 (d, J=11.0, 2H), 1.35-1.20 (br m, 5H).

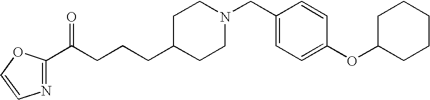

### Example 134

### 4-[1-(4-Cyclohexyloxy-benzyl)-piperidin-4-yl]-1-oxazol-2-yl-butan-1-one

HPLC: Rₜ=4.4 min. MS (ESI): mass calcd. for C₂₅H₃₄N₂O₃, 410.26; m/z found, 411.2 [M+H]⁺. ¹H NMR (CDCl₃): 7.82 (s, 1H), 7.32 (s, 1H), 7.29 (d, J=8.5, 2H), 6.87 (d, J=8.5, 2H), 4.23 (m, 1-H), 3.79 (s, 2H), 3.18 (d, J=11.5, 2H), 3.05 (t, J=7.5, 2H), 2.28 (d, J=11.5, 2H), 2.05 (s, 1H), 1.99-1.97 (br m, 2H), 1.83-1.71 (br m, 6H), 1.59-1.50 (br m, 5H), 1.41-1.26 (br m, 5H).

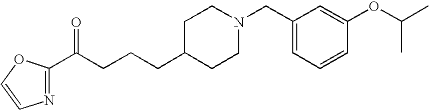

### Example 135

### 4-[1-(3-Isopropoxy-benzyl)-piperidin-4-yl]-1-oxazol-2-yl-butan-1-one

HPLC: Rₜ=4.1 min. MS (ESI): mass calcd. for C₂₂H₃₀N₂O₃, 370.23; m/z found, 371.2 [M+H]⁺. ¹H NMR (CDCl₃): 7.81 (s, 1H), 7.32 (s, 1H), 7.19 (t, J=8.0, 1H), 6.88-6.86 (br m, 2H), 6.78-6.76 (dd, J=2.0, 8.0, 1H), 4.60-4.52 (heptet, 1H), 3.46 (s, 2H), 3.05 (t, J=7.5, 2H), 2.88 (d, J=11.0, 2H), 1.96-1.92 (br m, 2H), 1.79-1.73 (m, 2H), 1.67-1.65 (br m, 2H), 1.32 (d, J=6, 6H), 1.34-1.27 (br m, 5H).

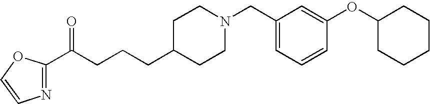

### Example 136

### 4-[1-(3-Cyclohexyloxy-benzyl)-piperidin-4-yl]-1-oxazol-2-yl-butan-1-one

HPLC: Rₜ=4.7 min. MS (ESI): mass calcd. for C₂₅H₃₄N₂O₃, 410.26; m/z found, 411.2 [M+H]⁺. ¹H NMR (CDCl₃): 7.81 (s, 1H), 7.32 (s, 1H), 7.18 (t, J=7.5, 1H), 6.89-6.85 (m, 2H), 6.78 (dd, J=2.0, 8.0, 1H), 4.25 (heptet, J=3.5, 1H), 3.45 (s, 2H), 3.05 (t, J=7.5, 2H), 2.88 (d, J=11.0, 2H), 1.99-1.91 (br m, 4H), 1.83-1.73 (br m, 4H), 1.37-1.65 (m, 2H), 1.60-1.54 (m, 1H), 1.52-1.47 (br m, 2H), 1.41-1.26 (br m, 8H).

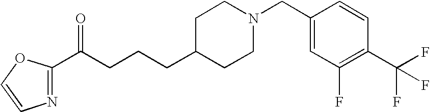

### Example 137

### 4-[1-(3-Fluoro-4-trifluoromethyl-benzyl)-piperidin-4-yl]-1-oxazol-2-yl-butan-1-one

HPLC: Rₜ=4.2 min. MS (ESI): mass calcd. for C₂₀H₂₂F₄N₂O₂, 398.16; m/z found, 399.1 [M+H]⁺. ¹H NMR (CDCl₃: 7.82 (s, 1H), 7.52 (t, J=8.0, 1H), 7.33 (s, 1H), 7.23-7.18 (m, 2H), 3.50 (s, 2H), 3.07 (t, J=7.5, 2H), 2.82 (d, J=11.5, 2H), 1.98 (t, J=11.5, 2H), 1.77 (m, 2H), 1.68 (d, J=11.0, 2H), 1.36-1.22 (m, 5H).

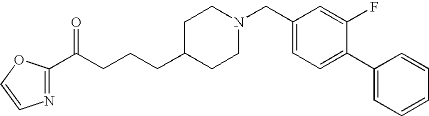

### Example 138

### 4-[1-(2-Fluoro-biphenyl-4-ylmethyl)-piperidin-4-yl]-1-oxazol-2-yl-butan-1-one

HPLC: Rₜ=4.4 min. MS (ESI): mass calcd. for C₂₅H₂₇FN₂O₂, 406.21; m/z found, 407.2 [M+H]⁺. ¹H NMR (CDCl₃): 7.81 (s, 1H), 7.55-7.54 (m, 2H), 7.48-7.42 (m, 2H), 7.39-7.34 (m, 2H), 7.32 (s, 1H), 7.16-7.14 (m, 2H), 3.51 (s, 2H), 3.06 (t, J=7.5, 2H), 2.90 (d, J=11.0, 2H), 2.00-1.96 (br m, 2H), 1.81-1.74 (br m, 2H), 1.701-1.682 (br m, 2H), 1.35-1.29 (br m, 5H).

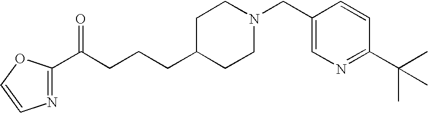

### Example 139

### 4-[1-(6-tert-Butyl-pyridin-3-ylmethyl)-piperidin-4-yl]-1-oxazol-2-yl-butan-1-one

HPLC: Rₜ=3.5 min. MS (ESI): mass calcd. for C₂₂H₃₁N₃O₂, 369.24; m/z found, 370.2 [M+H]⁺. ¹H NMR (CDCl₃): 8.44 (br d, J=2.0, 1H), 7.81 (br d, J=0.5, 1H), 7.60-7.58 (m, 1H), 7.22 (s, 1H), 7.29 (d, J=8.0, 1H), 3.46 (s, 2H), 3.05 (t, J=7.5, 2H), 2.86 (d, J=12.0), 2H), 1.95 (t, J=11.0, 2H), 1.79-1.73 (m, 2H), 1.66 (d, J=11.0, 2H), 1.361 (s, 9H), 1.36-1.23 (m, 5H).

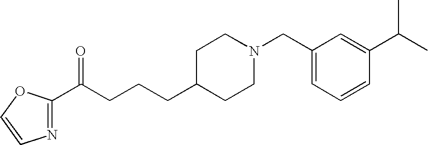

### Example 140

### 4-[1-(3-Isopropyl-benzyl)-piperidin-4-yl]-1-oxazol-2-yl-butan-1-one

HPLC: Rₜ=4.2 min: MS (ESI): mass calcd. for C₂₂H₃₀N₂O₂, 354.23; m/z found, 355.2 [M+H]⁺. ¹H NMR (CDCl₃): 7.80 (br d, J=0.5, 1H), 7.32 (s, 1H), 7.25-7.22 (m, 1H), 7.16 (s, 1H), 7.13-7.22 (m, 2H), 3.48 (s, 2H), 3.05 (t, J=7.5, 2H), 2.91-2.87 (br m, 3H), 1.95-1.91 (m, 2H), 1.79-1.73 (m, 2H), 1.67-1.65 (br m, 2H), 1.33-1.24 (br m, 5H), 1.25 (d, J=7.0, 6H).

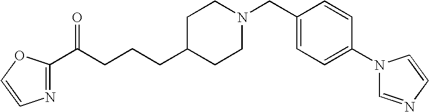

### Example 141

### 4-[1-(4-Imidazol-1-yl-benzyl)-piperidin-4-yl]-1-oxazol-2-yl-butan-1-one

The title compound was prepared and purified using methods analogous to those described in preceding examples, except that NaB(OAc)₃H was used in resin-bound form. HPLC: Rₜ=3.0 min. MS (ESI): mass calcd. for C₂₂H₂₆N₄₀₂, 378.21; m/z found, 379.2 [M+H]⁺. ¹H NMR (CDCl₃): 7.84 (s, 1H), 7.82 (s, 1H), 7.45-7.43 (m, 2H), 7.34-7.32 (m, 3H), 7.27 (m, 1H), 7.20 (br s, 1H), 3.53 (s, 2H), 3.06 (t, J=7.5, 2H), 2.88 (d, J=11.0, 2H), 2.00-1.96 (br m, 2H), 1.79-1.74 (m, 2H), 1.70-1.68 (m, 2H), 1.35-1.28 (m, 5H).

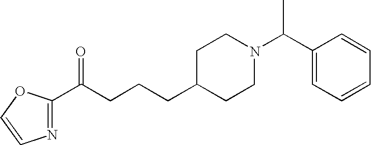

### Example 142

### 1-Oxazol-2-yl-4-[1-(1-phenyl-ethyl)-piperidin-4-yl]-butan-1-one

To a stirred solution of 1-oxazol-2-yl-4-piperidin-4-yl-butan-1-one hydrochloride (105 mg), KI (102.1 mg), and K₂CO₃ (170 mg) in CH₃CN (10.3 mL) was added (1-bromo-ethyl)-benzene (84 μL) and the resulting mixture was heated to reflux. After 24 h, the mixture was partitioned between EtOAc (100 mL) and satd. aq. NaHCO₃ (30 mL). The organic layer was dried (Na₂SO₄) and concentrated. Chromatographic purification (MeOH/CH₂Cl₂) afforded the title compound as a yellow solid (72.3 mg, 54%). HPLC: Rₜ=3.9 min. MS (ESI): mass calcd. for C₂₀H₂₆N₂O₂, 326.20; m/z found, 327.1 [M+H]⁺. ¹H NMR (CDCl₃): 7.80 (s, 1H), 7.31-7.29 (m, 5H), 7.24-7.21 (m, 1H), 3.40 (q, 1H), 3.05-3.02 (m, 3H), 2.81-2.78 (m, 1H), 1.95-1.90 (m, 1H), 1.84-1.80 (m, 1H), 1.77-1.68 (br m, 3H), 1.61-1.59 (br m, 1H), 1.38 (d, J=6.5, 3H), 1.32-1.28 (br m, 3H), 1.23-1.50 (br m, 2H).

The compounds in Examples 143-146 and 148-151 were purified by reversed phase preparative HPLC, using a Shimadzu HPLC with a Phenomenex Gemini C18 (5 μm, 150×21.2 mm) column. Detection was done at 2=254 nm. The flow rate was 30 mL/min. The gradient was 10 to 100% acetonitrile/water (0.05% trifluoroacetic acid) over 23 min.

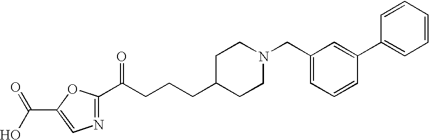

### Example 143

### 2-[4-(1-Biphenyl-3-ylmethyl-piperidin-4-yl)-butyryl]-oxazole-5-carboxylic acid trifluoroacetic acid salt

Step A. 2-(4-Piperidin-4-yl-butyryl)-oxazole-5-carboxylic acid hydrochloride. A suspension of 4-[4-(5-carboxy-oxazol-2-yl)-4-oxo-butyl]-piperidine-1-carboxylic acid tert-butyl ester (79 mg) in HCl (4 M in dioxane, 550 μL) was stirred for 2 h. The suspension was concentrated to afford the title compound as a white solid (60.7 mg, 91%). HPLC: Rₜ=3.1 min. MS (ESI): mass calcd. for C₁₃H₁₈N₂O₄, 266.13; m/z found, 267.1 [M−H]⁺. ¹H NMR (CD₃OD): 7.94 (s, 1H), 3.39-3.3.36 (m, 2H), 3.12 (t, J=7.5, 2H), 3.00-2.95 (m, 2H), 1.99-1.96 (m, 2H), 1.81-1.75 (m, 2H), 1.69-1.62 (br m, 1H), 1.43-1.36 (br m, 5H). Step B. To a stirred mixture of 2-(4-piperidin-4-yl-butyryl)-oxazole-5-carboxylic acid hydrochloride (37.8 mg), NEt₃ (36.8 μL), and biphenyl-3-carbaldehyde (22 μL) in MeOH (1:2 mL) was added Na(CN)BH₃ (8.3 mg). After 24 h, the mixture was concentrated. Purification via reversed phase chromatography afforded the title compound as a colorless oil (11.2 mg, 17%). HPLC: Rₜ=4.0 min. MS (ESI): mass calcd. for C₂₆H₂₈N₂O₄, 432.20; m/z found, 433.2 [M+H]⁺. ¹H NMR (CDCl₃): 11.18 (br s, 1H), 7.80 (s, 1H), 7.65-7.63 (m, 2H), 7.56-7.54 (m, 2H), 7.48-7.41 (br m, 3H), 7.38-7.35 (br m, 2H), 4.29 (s, 2H), 3.71 (d, J=12.0, 2H), 3.00 (t, J=7.5, 2H), 2.71-2.67 (m, 2H), 1.90-1.88 (m, 2H), 1.69-1.64 (m, 4H), 1.50-1.43 (br m, 1H), 1.35-1.31 (m, 2H).

The compounds in Examples 144-145 were prepared and purified using methods analogous to those described in preceding examples, substituting appropriate aldehyde reagents.

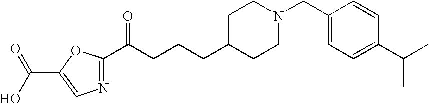

### Example 144

### 2-{4-[1-(4-Isopropyl-benzyl)-piperidin-4-yl]-butyryl}-oxazole-5-carboxylic acid trifluoroacetic acid salt

HPLC: Rₜ=4.1 min. MS (ESI): mass calcd. for C₂₃H₃₀N₂O₄, 398.22; m/z found, 399.2 [M+H]⁺. ¹H NMR (CDCl₃): 10.69 (br s, 1H), 7.83 (s, 1H), 7.31-7.27 (br m, 4H), 4.19 (s, 2H), 3.64 (d, J=10.5, 2H), 3.03 (t, J=7.0, 2H), 2.93-2.87 (m, 1H), 2.6-2.65 (br m, 2H), 1.91-1.88 (m, 2H), 1.71-1.62 (br m, 4H), 1.47 (br s, 1H), 1.35-1.32 (m, 2H), 1.23 (d, J=7.0, 6H).

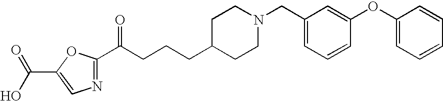

### Example 145

### 2-{4-[1-(3-Phenoxy-benzyl)-piperidin-4-yl]-butyryl}-oxazole-5-carboxylic acid trifluoroacetic acid salt

HPLC: Rₜ=4.3 min. MS (ESI): mass calcd. for C₂₆H₂₈N₂O₅, 448.20; m/z found, 449.1 [M+H]⁺. ¹H NMR (CDCl₃): 11.40 (br s, 1H), 7.81 (s, 1H), 7.36-7.32 (m, 3H), 7.15-7.11 (m, 2H), 7.01-6.92 (m, 4H), 4.19 (s, 2H), 3.66 (d, J=12.0, 2H), 3.04 (t, J=7.0, 2H), 2.66-2.62 (m, 2H), 1.90-1.87 (m, 2H), 1.73-1.63 (m, 4H), 1.49-1.43 (br m, 1H), 1.37-1.32 (m, 2H).

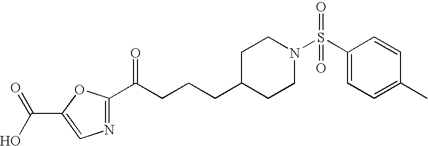

### Example 146

### 2-{4-[1-Toluene-4-sulfonyl)-piperidin-4-yl]-butyryl}-oxazole-5-carboxylic acid

To a mixture of 2-(4-piperidin-4-yl-butyryl)-oxazole-5-carboxylic acid hydrochloride (34.6 mg) and NEt₃ (79.4 μL) in CH₂Cl₂ (2.0 mL) was added 4-methyl-benzenesulfonyl chloride (32.6 mg). After 1 h, the mixture was concentrated. Purification by reversed phase chromatography afforded the title compound as a colorless oil (26.8 mg, 56%). HPLC: Rₜ=6.1 min. MS (ESI): mass calcd. for C₂₀H₂₄N₂O₆S, 420.14; m/z found, 421.1 [M+H]⁺. ¹H NMR (CDCl₃): 7.92 (s, 1H), 7.63 (d, J=8.5, 2H), 7.32 (d, J=8.0, 2H), 5.74 (br s, 1H), 3.75 (d, J=11.5, 2H), 3.05 (t, J=7.0, 2H), 2.43 (s, 3H), 2.23-2.18 (m, 2H), 1.74-1.68 (m, 4H), 1.33-1.71 (br m, 5H).

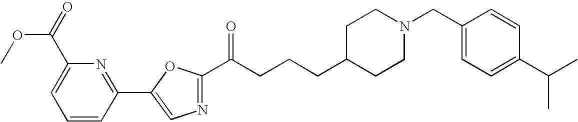

### Example 147

### 6-(2-[4-{1-(4-Isopropyl-benzyl)-piperidin-4-yl]-butyryl}-oxazol-5-yl)-pyridine-2-carboxylic acid methyl ester

Step A. 4-[4-(tert-Butyl-dimethyl-silanyloxy)-4-(5-tributylstannanyl-oxazol-2-yl)-butyl]-piperidine-1-carboxylic acid tert-butyl ester. To a stirred solution of 4-[4-(tert-butyl-dimethyl-silanyloxy)-4-oxazol-2-yl-butyl]-piperidine-1-carboxylic acid tert-butyl ester (2.21 g) in THF (39 mL) at −78° C. was added tert-butyllithium (1.7 M in pentanes, 3.26 mL), and the resulting solution was stirred for 30 min at −78° C. Tributyltin chloride (1.33 mL) was added, and the mixture was stirred for 90 min at −78° C. before warming to rt. After 30 min, the mixture was filtered through a short pad of silica gel (EtOAc/hexanes) and the filtrate was concentrated. Chromatographic purification (EtOAc/hexanes) afforded the title compound as a colorless oil (2.81 g, 77%). HPLC: Rₜ=5.7 min. MS (ESI): mass calcd. for C₃₅H₆₈N₂O₄SiSn, 728.40; m/z found, 729.3 [M+H]⁺. ¹H NMR (CDCl₃): 7.26 (s, 1H), 4.84-4.81 (m, 1H), 4.05 (br s, 2H), 2.64 (m, 2H), 1.93-1.77 (br m, 2H), 1.62-1.60 (br m, 3H), 1.57-1.51 (br m, 5H), 1.45 (m, 10H), 1.36-1.29 (br m, 8H), 1.26-1.22 (br m, 2H), 1.11-1.08 (m, 5H), 0.90-0.86 (br m, 18H), 0.05 (s, 3H), −0.09 (s, 3H).

Step B. 6-{2-[4-(1-tert-Butoxycarbonyl-piperidin-4-yl)-1-(tert-butyl-dimethyl-silanyloxy)-butyl]-oxazol-5-yl}-pyridine-2-carboxylic acid methyl ester. To a stirred solution of 4-[4-(tert-butyl-dimethyl-silanyloxy)-4-(5-tributylstannanyl-oxazol-2-yl)-butyl]-piperidine-1-carboxylic acid tert-butyl ester (1.04 g) in dioxane (17.4 mL) at 90° C. was added 6-bromo-pyridine-2-carboxylic acid methyl ester (600.8 mg) and Pd(PPh₃)₄ (321.2 mg). The solution was stirred for 1 h before cooling to rt. Chromatographic purification afforded the title compound as a colorless oil (609.4 mg, 76%). HPLC: Rₜ=9.6 min. MS (ESI): mass calcd. for C₃₀H₄₇N₃O₆Si, 573.32; m/z found, 574.3 [M+H]⁺. ¹H NMR (CDCl₃): 8.04 (dd, J=1.0, 7.5, 1H), 7.92 (t, J=8.0, 1H), 7.82 (dd, J=1.0, 8.0, 1H), 7.78 (s, 1H), 4.88-4.86 (m, 1H), 4.02 (s, 3H), 2.65 (br s, 2H), 1.99-1.84 (br m, 2H), 1.63-1.60 (br m, 3H), 1.53-1.47 (br m, 1H), 1.45 (s, 9H), 1.40-1.32 (br m, 2H), 1.30-1.25 (br m, 3H), 1.10-1.02 (m, 2H), 0.89 (s, 9H), 0.10 (s, 3H), −0.004 (s, 3H).

Step C. 6-{2-[4-(1-tert-Butoxycarbonyl-piperidin-4-yl)-1-hydroxy-butyl]-oxazol-5-yl}-pyridine-2-carboxylic acid methyl ester. To a stirred solution of 6-{2-[4-(1-tert-butoxycarbonyl-piperidin-4-yl)-1-(tert-butyl-dimethyl-silanyloxy)-butyl]-oxazol-5-yl}-pyridine-2-carboxylic acid methyl ester (817 mg) in THF (14.2 mL) at 0° C. was added tetrabutylammonium fluoride (4.3 mL). After 90 min, the solution was diluted with CH₂Cl₂ (100 mL) and washed with H₂O (10 mL). The organic layer was dried (Na₂SO₄) and concentrated. Chromatographic purification [(2 M NH₃/MeOH)/CH₂Cl₂] afforded the title compound as a white solid (645.1 mg, 98%). HPLC: Rₜ=5.9 min. MS (ESI): mass calcd. for C₂₄H₃₃N₃O₆, 459.24; m/z found, 360.1 [M-Boc+H]⁺. ¹H NMR (CDCl₃): 7.94 (dd, J=1.0, 7.5, 1H), 7.82 (t, J=8.0, 1H), 7.72 (dd, J=1.0, 7.5, 1H), 7.67 (s, 1H), 4.83-4.85 (m, 1H), 4.38 (d, J=5.0, 1H), 3.93 (s, 5H), 2.56 (br s, 2H), 1.94-1.86 (br m, 2H), 1.55-1.53 (m, 2H), 1.48-1.43 (m, 1H), 1.36 (s, 9H), 1.32-1.26 (br m, 1H), 1.23-1.16 (br m, 3H), 1.01-0.92 (m, 2H).

Step D. 6-{2-[4-(1-tert-Butoxycarbonyl-piperidin-4-yl)-butyryl]-oxazol-5-yl}-pyridine-2-carboxylic acid methyl ester. To a mixture of 6-{2-[4-(1-tert-butoxycarbonyl-piperidin-4-yl)-1-hydroxy-butyl]-oxazol-5-yl}-pyridine-2-carboxylic acid methyl ester (599.2 mg) in CH₂Cl₂ (13 mL) was added Dess-Martin periodinane (661.7 mg). After 1 h, the mixture was purified by chromatography which afforded the title compound as a yellow solid (589.8 mg, 99%). HPLC: Rₜ=6.7 min. MS (ESI): mass calcd. for C₂₄H₃₁N₃O₆, 457.22; m/z found, 358.1 [M-Boc+H]⁺. ¹H NMR (CDCl₃): 8.13-8.11 (m, 1H), 8.05-7.96 (br m, 3H), 4.13-4.04 (m, 5H), 3.12 (t, J=7.0, 2H), 2.68 (m, 2H), 2.26 (s, 1H), 1.84-1.78 (m, 2H), 1.69-1.67 (m, 2H), 1.45 (s, 9H), 1.37-1.33 (br m, 2H), 1.43-1.06 (m, 2H).

Step E. 6-[2-(4-Piperidin-4-yl-butyryl)-oxazol-5-yl]-pyridine-2-carboxylic acid methyl ester hydrochloride. To a solution of 6-{2-[4-(1-tert-butoxycarbonyl-piperidin-4-yl)-butyryl]-oxazol-5-yl}-pyridine-2-carboxylic acid methyl ester (600 mg) in CH₂Cl₂ (4.4 mL) was added HCl (4 M in dioxane, 1.6 mL). After 3 h, the mixture was concentrated to afford the title compound as a yellow solid (462.8 mg, 90%). HPLC: Rₜ=3.7 min. MS (ESI): mass calcd. for C₁₉H₂₃N₃O₄, 357.17; m/z found, 358.1 [M+H]⁺. ¹H NMR (CD₃OD): 8.16-8.10 (br m, 4H), 4.02 (s, 3H), 3.66 (s, 1H), 3.39-3.37 (m, 2H), 3.16 (t, J=7.5, 2H), 3.01-2.95 (m, 2H), 2.01-1.98 (m, 2H), 1.85-1.79 (m, 2H), 1.45-1.34 (br m, 5H).

Step F. To stirred mixture of 6-[2-(4-piperidin-4-yl-butyryl)-oxazol-5-yl]-pyridine-2-carboxylic acid methyl ester hydrochloride (59.1 mg), NEt₃ (23.0 μL), and 4-isopropyl-benzaldehyde (25.0 μL) in CH₂Cl₂ (1.5 mL) was added NaB(OAc)₃H (35.0 mg). After 3 h, the mixture was diluted with CH₂Cl₂ (10 mL) and 1 N NaOH (1 mL) and then extracted using a Varian Chemelut Solid-Liquid Extraction cartridge. The organic filtrate was concentrated. Chromatographic purification (MeOH/CH₂Cl₂) afforded the title compound as a white solid (43.0 mg, 59%). HPLC: Rₜ=4.4 min. MS (ESI): mass calcd. for C₂₉H₃₅N₃O₄, 489.26 m/z found, 490.2 [M+H]⁺. ¹H NMR (CDCl₃): 8.13-8.11 (m, 1H), 8.05-7.96 (br m, 2H), 7.32-7.20 (br m, 5H), 4.04 (s, 2H), 3.76 (s, 1H), 3.48 (s, 1H), 3.16-3.08 (br m, 4H), 2.91 (heptet, J=6.8, 1H), 2.24 (t, J=11.2, 2H), 2.04 (s, 1H), 1.82-1.74 (br m, 4H), 1.57-1.49 (br m, 2H), 1.39-1.36 (m, 3H), 1.25 (d, J=6.8, 6H).

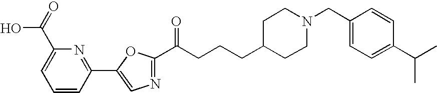

### Example 148

### 6-(2-{4-[1-(4-isopropyl-benzyl)-piperidin-4-yl]-butyryl}-oxazol-5-yl)-pyridine-2-carboxylic acid trifluoroacetic acid salt

To a 0° C. mixture of 6-(2-{4-[1-(4-isopropyl-benzyl)-piperidin-4-yl]-butyryl}-oxazol-5-yl)-pyridine-2-carboxylic acid methyl ester (35.5 mg) was added KOH (10.2 mg) in MeOH (1 mL). After 24 h, the mixture of purified by reversed phase chromatography to afford the title compound as a colorless oil (29.0 mg, 84%). HPLC: Rₜ=4.2 min. MS (ESI): mass calcd. for C₂₈H₃₃N₃O₄, 475.25; m/z found, 476.2 [M+H]⁺. ¹H NMR (CDCl₃): 11.95 (br s, 1H), 9.39 (br s, 1H), 8.25-8.23 (m, 1H), 8.09 (m, 1H), 7.96 (m, 1H), 7.32-7.26 (br m, 5H), 4.17 (s, 2H), 3.62-3.61 (m, 2H), 3.10 (t, J=7.0, 2H), 2.92 (heptet, J=7.0, 1H), 2.61 (br s, 2H), 1.91-1.88 (m, 2H), 1.80-1.70 (br m, 4H), 1.47-1.39 (br m, 3H), 1.24 (d, J=7.0, 6H).

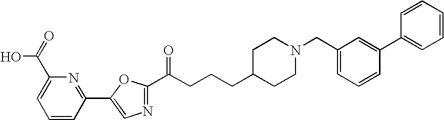

### Example 149

### 6-{2-[4-(1-Biphenyl-3-ylmethyl-piperidin-4-yl)-butyryl]-oxazol-5-yl}-pyridine-2-carboxylic acid trifluoroacetic acid salt

HPLC: Rₜ=4.2 min. MS (ESI): mass calcd. for C₃₁H₃₁N₃O₄, 509.23; m/z found, 510.2 [M+H]⁺. ¹H NMR (CDCl₃): 11.97 (br s, 1H), 8.89 (br s, 1H), 8.22-8.21 (m, 1H), 8.06-8.05 (m, 2H), 7.96-7.94 (m, 1H), 7.67-7.65 (m, 2H), 7.57-7.56 (m, 2H), 7.50-7.42 (br m, 3H), 7.38-7.35 (m, 2H), 4.27 (br s, 2H), 3.68-3.66 (m, 2H), 3.09 (t, J=7.0, 2H), 2.67 (m, 2H), 1.92-189 (m, 2H), 1.78-1.71 (br m, 4H), 1.49 (br s, 1H), 1.41-1.38 (br m, 2H).

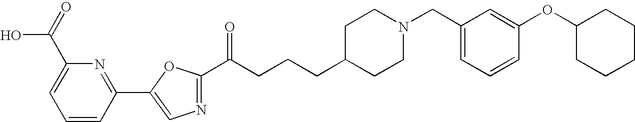

### Example 150

### 6-(2-{4-[1-(3-Cyclohexyloxy-benzyl)-piperidin-4-yl]-butyryl}-oxazol-5-yl)-pyridine-2-carboxylic acid trifluoroacetic acid salt

HPLC: Rₜ=4.4 min. MS (ESI): mass calcd. for C₃₁H₃₇N₃O₅, 531.27; m/z found, 532.2 [M+H]⁺. ¹H NMR (CDCl₃): 11.56 (br s, 1H), 8.68 (br s, 1H), 8.25-8.23 (m, 1H), 8.10-8.08 (m, 2H), 7.97-7.95 (m, 1H), 7.30-7.29 (m, 1H), 6.97-6.94 (m, 2H), 6.89 (d, J=7.5, 1H), 4.26 (t, J=5.0, 1H), 4.16 (s, 2H), 3.62 (s, J=11.0, 2H), 3.11 (t, J=7.5, 2H), 2.63 (s, 2H), 1.96-1.89 (br m, 4H), 1.79-1.76 (br m, 4H), 1.71-1.66 (br m, 2H), 1.59-1.56 (m, 1H), 1.54-1.47 (br m, 3H), 1.42-1.37 (br m, 3H), 1.35-1.26 (br m, 2H).

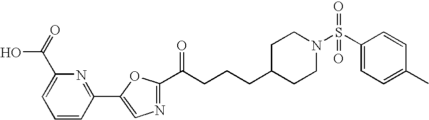

### Example 151

### 6-(2-{4-[1-(Toluene-4-sulfonyl)-piperidin-4-yl]-butyryl}-oxazol-5-yl)-pyridine-2-carboxylic acid trifluoroacetic acid salt

Step A. 6-(2-{4-[1-(Toluene-4-sulfonyl)-piperidin-4-yl]-butyryl}-oxazol-5-yl)-Pyridine-2-carboxylic acid methyl ester. To a mixture of 6-[2-(4-piperidin-4-yl-butyryl)-oxazol-5-yl]-pyridine-2-carboxylic acid methyl ester hydrochloride (62.0 mg) and NEt₃ (33.6 μL) in CH₂Cl₂ (1.6 mL) was added 4-methyl-benzenesulfonyl chloride (33.6 mg). After 1 h, the mixture was diluted with CH₂Cl₂ (10 mL) and 1 N NaOH (1 mL) and then extracted using a Varian Chemelut Solid-Liquid Extraction cartridge. The organic filtrate was concentrated. Chromatographic purification (EtOAc/CH₂Cl₂) afforded the title compound as a white solid (49.7 mg, 61%). HPLC: Rₜ=6.4 min. MS (ESI): mass calcd. for C₂₆H₂₉N₃O₆S, 511.18, m/z found, 512.1 [M+H]⁺. ¹H NMR (CDCl₃): 8.12 (dd, J=1.0, 7.5, 1H), 8.03-7.96 (m, 3H), 7.64 (d, J=8.0, 2H), 7.32 (d, J=8.5, 2H), 4.03 (s, 2H), 3.78-3.75 (m, 2H), 3.08 (t, J=7.5, 2H), 2.44 (s, 3H), 2.24-2.19 (m, 2H), 1.78-1.72 (m, 4H), 1.35-1.21 (m, 6H).

Step B. To a stirred mixture of 6-(2-{4-[1-(toluene-4-sulfonyl)-piperidin-4-yl]-butyryl}-oxazol-5-yl)-pyridine-2-carboxylic acid methyl ester (49.7 mg) was added KOH (13.6 mg) in MeOH (1 mL). The mixture was sonicated and heated to 50° C. After 3 h, the mixture was allowed to cool to rt. Purification by reversed phase chromatography afforded the title compound as a white solid (29.0 mg, 49%). HPLC: Rₜ=6.0 min. MS (ESI): mass calcd. for C₂₅H₂₇N₃O₆S, 497.16; m/z found, 498.1 [M+H]⁺. ¹H NMR (CDCl₃): 8.27-8.25 (m, 1H), 8.13-8.09 (br m, 2H), 7.92 (s, 1H), 7.50 (d, J=8.0, 2H), 7.32 (d, J=8.0, 2H), 3.79-3.76 (m, 2H), 3.09 (t, J=7.0, 2H), 2.44 (s, 3H), 2.24-2.20 (m, 2H), 1.79-1.74 (br m, 4H), 1.36-1.24 (br m, 5H).

### Biological Testing:

### Assay Method 1

A. Transfection of Cells with Human FAAH

A 10-cm tissue culture dish with a confluent monolayer of SK-N-MC cells was split 2 days (d) prior to transfection. Using sterile technique, the media was removed and the cells were detached from the dish by the addition of trypsin. One fifth of the cells were then placed onto a new 10-cm dish. Cells were grown in a 37° C. incubator with 5% CO₂ in Minimal Essential Media Eagle with 10% Fetal Bovine Serum. After 2 d, cells were approximately 80% confluent. These cells were removed from the dish with trypsin and pelleted in a clinical centrifuge. The pellet was re-suspended in 400 μL complete media and transferred to an electroporation cuvette with a 0.4 cm gap between the electrodes. Supercoiled human FAAH cDNA (1 μg) was added to the cells and mixed. The voltage for the electroporation was set at 0.25 kV, and the capacitance was set at 960 μF. After electroporation, the cells were diluted into complete media (10 mL) and plated onto four 10-cm dishes. Because of the variability in the efficiency of electroporation, four different concentrations of cells were plated. The ratios used were 1:20, 1:10, and 1:5, with the remainder of the cells being added to the fourth dish. The cells were allowed to recover for 24 h before adding the selection media (complete media with 600 μg/mL G418). After 10 d, dishes were analyzed for surviving colonies of cells. Dishes with well-isolated colonies were used. Cells from individual colonies were isolated and tested. The clones that showed the most FAAH activity, as measured by anandamide hydrolysis, were used for further study.

### B. FAAH Assay

T84 frozen cell pellets or transfected SK-N-MC cells (contents of 1×15 cm culture dishes) were homogenized in 50 mL of FAAH assay buffer (125 mM Tris, 1 mM EDTA, 0.2% Glycerol, 0.02% Triton X-100, 0.4 mM Hepes, pH 9). The assay mixture consisted of 50 mL of the cell homogenate, 10 μL of the test compound, and 40 μL of anandamide [1-³H-ethanolamine] (3H-AEA, Perkin-Elmer, 10.3 Cᵢ/mmol), which was added last, for a final tracer concentration of 80 nM. The reaction mixture was incubated at rt for 1 h. During the incubation, 96-well Multiscreen filter plates (catalog number MAFCNOB50; Millipore, Bedford, Mass., USA) were loaded with 25 μL of activated charcoal (Multiscreen column loader, catalog number MACL09625, Millipore) and washed once with 100 μL of MeOH. Also during the incubation, 96-well DYNEX MicroLite plates (catalog number NL510410) were loaded with 100 μL of MicroScint40 (catalog number 6013641, Packard Bioscience, Meriden, Conn., USA). After the 1 h incubation, 60 μL of the reaction mixture were transferred to the charcoal plates, which were then assembled on top of the DYNEX plates using Centrifuge Alignment Frames (catalog number MACF09604, Millipore). The unbound labeled ethanolamine was centrifuged through to the bottom plate (5 min at 2000 rpm), which was preloaded with the scintillant, as described above. The plates were sealed and left at rt for 1 h before counting on a Hewlett Packard TopCount. Results for compounds tested in this assay are presented in Table 1.

### Assay Method 2

A. Transfection of Cells with Rat FAAH

A 10-cm tissue culture dish with a confluent monolayer of SK-N-MC cells was split 2 days (d) prior to transfection. Using sterile technique, the media was removed and the cells were detached from the dish by the addition of trypsin. One fifth of the cells were then placed onto a new 10-cm dish. Cells were grown in a 37° C. incubator with 5% CO₂ in Minimal Essential Media Eagle with 10% Fetal Bovine Serum. After 2 d, cells were approximately 80% confluent. These cells were removed from the dish with trypsin and pelleted in a clinical centrifuge. The pellet was re-suspended in 400 μL complete media and transferred to an electroporation cuvette with a 0.4 cm gap between the electrodes. Supercoiled rat FAAH cDNA (1 μg) was added to the cells and mixed. The voltage for the electroporation was set at 0.25 kV, and the capacitance was set at 960 μF. After electroporation, the cells were diluted into complete media (10 mL) and plated onto four 10-cm dishes. Because of the variability in the efficiency of electroporation, four different concentrations of cells were plated. The ratios used were 1:20, 1:10, and 1:5, with the remainder of the cells being added to the fourth dish. The cells were allowed to recover for 24 h before adding the selection media (complete media with 600 μg/mL G418). After 10 d, dishes were analyzed for surviving colonies of cells. Dishes with well-isolated colonies were used. Cells from individual colonies were isolated and tested. The clones that showed the most FAAH activity, as measured by anandamide hydrolysis, were used for further study.

### B. FAAH Assay

T84 frozen cell pellets or transfected SK-N-MC cells (contents of 1×15 cm culture dishes) were homogenized in 50 mL of FAAH assay buffer (125 mM Tris, 1 mM EDTA, 0.2% Glycerol, 0.02% Triton X-100, 0.4 mM Hepes, pH 9). The assay mixture consisted of 50 μL of the cell homogenate, 10 μL of the test compound, and 40 μL of anandamide [1-³H-ethanolamine] (3H-AEA, Perkin-Elmer, 10.3 Cᵢ/mmol), which was added last, for a final tracer concentration of 80 nM. The reaction mixture was incubated at rt for 1 h. During the incubation, 96-well Multiscreen filter plates (catalog number MAFCNOB50; Millipore, Bedford, Mass., USA) were loaded with 25 μL of activated charcoal (Multiscreen column loader, catalog number MACL09625, Millipore) and washed once with 100 μL of MeOH. Also during the incubation, 96-well DYNEX MicroLite plates (catalog number NL510410) were loaded with 100 μL of MicroScint40 (catalog number 6013641, Packard Bioscience, Meriden, Conn., USA). After the 1 h incubation, 60 μL of the reaction mixture were transferred to the charcoal plates, which were then assembled on top of the DYNEX plates using Centrifuge Alignment Frames (catalog number MACF09604, Millipore). The unbound labeled ethanolamine was centrifuged through to the bottom plate (5 min at 2000 rpm), which was preloaded with the scintillant, as described above. The plates were sealed and left at rt for 1 h before counting on a Hewlett Packard TopCount. Results for compounds tested in this assay are presented in Table 1.

TABLE 1

| | Assay 1 | Assay 2 |
|---|---|---|
| Ex. | IC₅₀ (nM) | IC₅₀ (nM) |
| 1 | 1.8 | 27 |
| 2 | 0.4 | 4.7 |
| 3 | 315 | 900 |
| 4 | 3000 | 10000 |
| 5 | 1200 | 3000 |
| 6 | 359 | 550 |
| 7 | 850 | 1000 |
| 8 | 100 | 350 |
| 9 | 66 | 354 |
| 10 | 60 | 80 |
| 11 | 36 | 95 |
| 12 | 50 | 70 |
| 13 | 50 | 180 |
| 14 | 86 | 1000 |
| 15 | 4 | 2.4 |
| 16 | 132 | 663 |
| 17 | 36 | 308 |
| 18 | 400 | 1000 |
| 19 | 90 | 305 |
| 20 | 8 | 9 |
| 21 | 29 | 86 |
| 22 | 3 | 5 |
| 23 | 14 | 31 |
| 24 | 32 | 18 |
| 25 | 90 | 320 |
| 26 | 400 | 900 |
| 27 | 500 | 1300 |
| 28 | 67 | 563 |
| 29 | 45 | 180 |
| 30 | 250 | 2250 |
| 31 | 106 | 288 |
| 32 | 28 | 112 |
| 33 | 848 | 889 |
| 34 | 240 | 250 |
| 35 | 19 | 38 |
| 36 | 46 | 56 |
| 37 | 22 | 18 |
| 38 | 100 | 100 |
| 39 | 834 | 463 |
| 40 | 43 | 75 |
| 41 | 640 | 712 |
| 42 | 430 | 800 |
| 43 | 4000 | 10000 |
| 44 | 40 | 10 |
| 45 | 109 | 130 |
| 46 | 12 | 3 |
| 47 | 400 | 700 |
| 48 | 100 | 216 |
| 49 | 100 | 350 |
| 50 | 2 | 10 |
| 51 | 230 | 635 |
| 52 | 1043 | 13 |
| 53 | 600 | 800 |
| 54 | 6 | 125 |
| 55 | 905 | 95 |
| 56 | 142 | 3350 |
| 57 | 120 | 1600 |
| 58 | 60 | 720 |
| 59 | 705 | 165 |
| 60 | 4 | 34 |
| 61 | 2 | 37 |
| 62 | 14 | 110 |
| 63 | 3 | 2 |
| 64 | 6 | 14 |
| 65 | 69 | 240 |
| 66 | 0.7 | 2 |
| 67 | 50 | 118 |
| 68 | 10 | 55 |
| 69 | 4 | 9 |
| 70 | 7 | 19 |
| 71 | 7 | 49 |
| 72 | 0.9 | 2 |
| 73 | 24 | 45 |
| 74 | 1300 | 5000 |
| 75 | 600 | 1000 |
| 76 | 400 | 650 |
| 77 | 313 | 1000 |
| 78 | 98 | 500 |
| 79 | 10000 | 10000 |
| 80 | 1200 | 2000 |
| 81 | 200 | 1000 |
| 82 | 23 | 49 |
| 83 | 7 | 84 |
| 84 | 260 | 563 |
| 85 | 315 | 240 |
| 86 | 9500 | 10000 |
| 87 | 4000 | 10000 |
| 88 | 325 | 3000 |
| 89 | 130 | 700 |
| 90 | 10 | 118 |
| 91 | 45 | 270 |
| 92 | 60 | 290 |
| 93 | 70 | 923 |
| 94 | 400 | 2100 |
| 95 | 10000 | 1200 |
| 96 | 125 | 132 |
| 97 | 12 | 23 |
| 98 | 0.4 | 7 |
| 99 | 5 | 8 |
| 100 | 30 | 11 |
| 101 | 23 | 44 |
| 102 | 530 | 3000 |
| 103 | 108 | 250 |
| 104 | 4 | 48 |
| 105 | 53 | 57 |
| 106 | 2000 | 10000 |
| 107 | 180 | 2000 |
| 108 | 23 | 235 |
| 109 | 55 | 528 |
| 110 | 9 | 141 |
| 111 | 15 | 193 |
| 112 | 14 | 174 |
| 113 | 28 | 318 |
| 114 | 110 | 325 |
| 115 | 78 | 750 |
| 116 | 370 | 7000 |
| 117 | 175 | 277 |
| 118 | 354 | 935 |
| 119 | 0.8 | 3.0 |
| 120 | 2 | 21 |
| 121 | 258 | 323 |
| 123 | 200 | 280 |
| 124 | 30 | 450 |
| 125 | 4 | 81 |
| 126 | 23 | 810 |
| 127 | 8 | 57 |
| 128 | 10000 | 10000 |
| 129 | 10000 | 10000 |
| 130 | 30 | 19 |
| 131 | 88 | 760 |
| 132 | 52 | 950 |
| 133 | 460 | 2000 |
| 134 | 119 | 119 |
| 135 | 5 | 9 |
| 136 | 1 | 2 |
| 137 | 10 | 380 |
| 138 | 71 | 113 |
| 139 | 50 | 550 |
| 140 | 1 | 3 |
| 141 | 84 | 1500 |
| 142 | 190 | 3000 |
| 143 | 14 | 3 |
| 144 | 75 | 17 |
| 145 | 6 | 3 |
| 146 | 26 | 14 |
| 147 | 18 | 8 |
| 148 | 60 | 56 |
| 149 | 9 | 2 |
| 150 | 16 | 7 |
| 151 | 21 | 25 |

While the invention has been illustrated by reference to exemplary and preferred embodiments, it will be understood that the invention is intended not to be limited to the foregoing detailed description, but to be defined by the appended claims as properly construed under principles of patent law.

What is claimed is:

1. A compound of Formula (I): 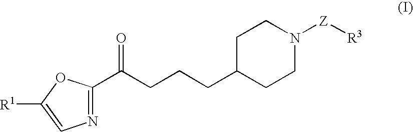
wherein:
R¹ is —H; a —CO₂C₁₋₄alkyl or —CO₂H group; or a phenyl, pyridyl, pyridazinyl, pyrimidinyl, pyrazinyl, thiophenyl, furanyl, or oxazolyl group, each unsubstituted or substituted with —CO₂H or —CO₂C₁₋₄alkyl;
Z is —C(O)—, —CO₂—, —SO₂—, —C(O)NH—, —CH₂—, or —CH(CH₃)—; and
R³ is:
(a) —(CH₂)ₙ—R⁴, where n is 0, 1, or 2, and R⁴ is:
(i) phenyl, unsubstituted or substituted with one, or two, or three R^{a} moieties or where two adjacent R^{a} moieties together form —O(CH₂)₁₋₂O— or —O(CF₂)O—;
where each R^{a} moiety is —C₁₋₇alkyl, —C₃₋₇cycloalkyl, —C₂₋₇alkenyl, —OH, —OC₁₋₇alkyl, —OC₃₋₇cycloalkyl, phenyl unsubstituted or substituted with R^{b}, phenoxy unsubstituted or substituted with R^{b}, furanyl, thiophenyl, imidazolyl, fluoro, chloro, bromo, iodo, —CF₃, —OCF₃, —SC₁₋₄alkyl, —SO₂C₁₋₄alkyl, —SOC₁₋₄alkyl, —CN, —CO₂C₁₋₄alkyl, —CO₂H, —COC₁₋₄alkyl, —SO₂NR^{c}R^{d}, —NR^{c}SO₂R^{d}, —C(O)NR^{c}R^{d}, —NR^{c}C(O)R^{d}, or —N(R^{c})R^{d};
where R^{b} is selected from the group consisting of —C₁₋₄alkyl, —OC₁₋₄alkyl, fluoro, chloro, bromo, iodo, —CN, —OH, —CF₃, —OCF₃, and —NO₂; and
where R^{c} and R^{d} are each independently —H or —C₁₋₇alkyl, or R^{c} and R^{d} taken together form a 3- to 7-membered heterocycloalkyl ring;
(ii) a five- or six-membered monocyclic heteroaryl ring, unsubstituted or substituted with one or two R^{a} moieties as defined above;
(iii) naphthyl, unsubstituted or substituted one or two R^{e} moieties, where each R^{e} moiety is independently selected from the group consisting of —C₁₋₄alkyl, —OC₁₋₄alkyl, fluoro, chloro, bromo, iodo, —CN, —OH, —CF₃, —OCF₃, and —NO₂;
(iv) a nine- or ten-membered fused bicyclic heteroaryl, unsubstituted or substituted with one or two R^{e} moieties; or
(v) —C₃₋₉cycloalkyl;
(b) —(CH₂)ₓO(CH₂)_{y}R⁴, where when Z is —C(O)—, —SO₂—, or —CH₂—, x is 1 or 2 and y is 0, 1, or 2, and when Z is —CO₂— or —C(O)NH—, x is 2 and y is 0, 1, or 2;
(c) —C₂₋₉alkyl; or
(d) —C₂₋₉alkyl, where one carbon chain member is replaced by nitrogen or oxygen;
or a pharmaceutically acceptable salt, pharmaceutically acceptable prodrug, or pharmaceutically active metabolite of such compound.

2. A compound, pharmaceutically acceptable salt, pharmaceutically acceptable prodrug, or pharmaceutically active metabolite as defined in claim 1, wherein R¹ is —H.

3. A compound, pharmaceutically acceptable salt, pharmaceutically acceptable prodrug, or pharmaceutically active metabolite as defined in claim 1, wherein R¹ is a —CO₂CH₃ or —CO₂H group.

4. A compound, pharmaceutically acceptable salt, pharmaceutically acceptable prodrug, or pharmaceutically active metabolite as defined in claim 1, wherein R¹ is a pyridyl group, unsubstituted or substituted with —CO₂H.

5. A compound, pharmaceutically acceptable salt, pharmaceutically acceptable prodrug, or pharmaceutically active metabolite as defined in claim 1, wherein R³ is R⁴, and R⁴ is tert-butyl, phenyl or pyridyl.

6. A compound, pharmaceutically acceptable salt, pharmaceutically acceptable prodrug, or pharmaceutically active metabolite as defined in claim 1, wherein each R³ moiety is independently methyl, isopropyl, tert-butyl, isopropoxy, cyclohexyloxy, phenyl, phenoxy, 1H-imidazol-1-yl, fluoro, chloro, or CF₃.

7. A compound of Formula (IA): 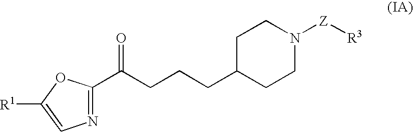
wherein:
R¹ is —H, or a phenyl, pyridyl, pyridazinyl, pyrimidinyl, pyrazinyl, thiophenyl, furanyl, or oxazolyl group;
Z is —C(O)—, —CO₂—, —SO₂—, —C(O)NH—, or —CH₂—; and
R³ is:
(a) —(CH₂)ₙ—R⁴, where n is 0, 1, or 2, and R⁴ is:
(i) phenyl, unsubstituted or substituted with one, or two, or three R^{a} moieties or where two adjacent R^{a} moieties together form —O(CH₂)₁₋₂O— or —O(CF₂)O—;
where each R^{a} moiety is —C₁₋₇alkyl, —C₃₋₇cycloalkyl, —C₂₋₇alkenyl, —OH, —OC₁₋₇alkyl, phenyl unsubstituted or substituted with R^{b}, phenoxy unsubstituted or substituted with R^{b}, furanyl, thiophenyl, fluoro, chloro, bromo, iodo, —CF₃, —OCF₃, —SC₁₋₄alkyl, —SO₂C₁₋₄alkyl, —SOC₁₋₄alkyl, —CN, —CO₂C₁₋₄alkyl, —CO₂H, —COC₁₋₄alkyl, —SO₂NR^{c}R^{d}, —NR^{c}SO₂R^{d}, —C(O)NR^{c}R^{d}, —NR^{c}C(O)R^{d}, or —N(R^{c})R^{d};
where R^{b} is selected from the group consisting of —C₁₋₄alkyl, —OC₁₋₄alkyl, fluoro, chloro, bromo, iodo, —CN, —OH, —CF₃, —OCF₃, and —NO₂; and
where R^{c} and R^{d} are each independently —H or —C₁₋₇alkyl, or R^{c} and R^{d} taken together form a 3- to 7-membered heterocycloalkyl ring;
(ii) a five- or six-membered monocyclic heteroaryl ring, unsubstituted or substituted with one or two R^{a} moieties as defined above;
(iii) naphthyl, unsubstituted or substituted one or two R^{e} moieties, where each R^{e} moiety is independently selected from the group consisting of —C₁₋₄alkyl, —OC₁₋₁₄alkyl, fluoro, chloro, bromo, iodo, —CN, —OH, —CF₃, —OCF₃, and —NO₂;
(iv) a nine- or ten-membered fused bicyclic heteroaryl, unsubstituted or substituted with one or two R^{e} moieties; or
(v) —C₃₋₉cycloalkyl;
(b) —(CH₂)ₓO(CH₂)_{y}R⁴ where when Z is —C(O)—, —SO₂—, or —CH₂—, x is 1 or 2 and y is 0, 1, or 2, and when Z is —CO₂— or —C(O)NH—, x is 2 and y is 0, 1, or 2;
(c) —C₂₋₉alkyl; or
(d) —C₂₋₉alkyl, where one carbon chain member is replaced by nitrogen or oxygen;
or a pharmaceutically acceptable salt, pharmaceutically acceptable prodrug, or pharmaceutically active metabolite of such compound.

8. A compound, pharmaceutically acceptable salt, pharmaceutically acceptable prodrug, or pharmaceutically active metabolite as defined in claim 7, wherein R¹ is —H, 2-pyridyl, or 2-furanyl.

9. A compound, pharmaceutically acceptable salt, pharmaceutically acceptable prodrug, or pharmaceutically active metabolite as defined in claim 7, wherein Z is —C(O)—, —CO₂—, —SO₂—, or —CH₂—.

10. A compound, pharmaceutically acceptable salt, pharmaceutically acceptable prodrug, or pharmaceutically active metabolite as defined in claim 7, wherein Z is —C(O)— or —CH₂—.

11. A compound, pharmaceutically acceptable salt, pharmaceutically acceptable prodrug, or pharmaceutically active metabolite as defined in claim 7, wherein R³ is —R⁴, —CH₂—R⁴, —(CH₂)₂—R⁴, —CH₂—O—R⁴, —CH₂—O—CH₂—R⁴, —CH₂—O—R⁴, —CH₂—O—CH₂—R⁴, —CH₂—O—CH₂CH₂—R⁴, —CH₂CH₂—O—R⁴, —CH₂CH₂—O—CH₂—R⁴, or —CH₂CH₂—O—CH₂CH₂—R⁴, wherein R⁴ is phenyl, pyridyl, pyridazinyl, pyrimidinyl, pyrazinyl, pyrrolyl, thiophenyl, furanyl, pyrazolyl, imidazolyl, oxazolyl, isoxazolyl, thiazolyl, oxadiazolyl, thiadiazolyl, naphthyl, cyclopropyl, cyclobutyl, cyclopentyl, cyclohexyl, cycloheptyl, cyclooctyl, or cyclononyl.

12. A compound, pharmaceutically acceptable salt, pharmaceutically acceptable prodrug, or pharmaceutically active metabolite as defined in claim 7, wherein R³ is ethyl, propyl, isopropyl, 2-methylpropyl, 2,2-dimethylpropyl, 1,2-dimethylpropyl, butyl, isobutyl, sec-butyl, tert-butyl, 2-methylbutyl, pentyl, isopentyl, hexyl, octyl, hydroxyethyl, hydroxypropyl, methoxyethyl, ethoxyethyl, methoxypropyl, methoxybutyl, aminoethyl, 2-methylaminoethyl, or 2-methylaminopropyl.

13. A compound, pharmaceutically acceptable salt, pharmaceutically acceptable prodrug, or pharmaceutically active metabolite as defined in claim 7, wherein R³ is —R⁴, —CH₂—R⁴, —(CH₂)₂—R⁴, —CH₂—O—R⁴, —CH₂—O—CH₂—R⁴, —CH₂—O—R⁴, —CH₂—O—CH₂—R⁴, —CH₂—O—CH₂CH₂—R⁴, —CH₂CH₂—O—R⁴, —CH₂CH₂—O—CH₂—R⁴, or —CH₂CH₂—O—CH₂CH₂—R⁴, wherein R⁴ is phenyl, pyridyl, isoxazolyl, furanyl, naphthyl, quinolinyl, quinoxalinyl, naphthyridinyl, cyclopentyl, or cyclohexyl.

14. A compound, pharmaceutically acceptable salt, pharmaceutically acceptable prodrug, or pharmaceutically active metabolite as defined in claim 7, wherein R³ is ethyl, propyl, isopropyl, 2,2-dimethylpropyl, butyl, isobutyl, sec-butyl, tert-butyl, hexyl, octyl, 3-methylbutyl, methoxyethyl, or ethoxyethyl.

15. A compound, pharmaceutically acceptable salt, pharmaceutically acceptable prodrug, or pharmaceutically active metabolite as defined in claim 7, wherein R³ is phenyl, 3-phenoxyphenyl, 4-phenoxyphenyl, 4-fluorophenyl, 3-fluorophenyl, 4-chlorophenyl, 3-chlorophenyl, benzo[1,3]dioxolyl, 4-methylphenyl, 3-methylphenyl, 4-t-butoxyphenyl, 2-methylphenyl, 2,3-difluorophenyl, 4-isobutylphenyl, 4-t-butylphenyl, 3,4-dibromophenyl, 3,4-dichlorophenyl, 3-chloro-4-fluorophenyl, 4-methoxyphenyl, 3-methoxyphenyl, 4-isopropylphenyl, 4-isopropoxyphenyl, 4-ethylphenyl, 3-biphenyl, 4-biphenyl, 2-chlorophenyl, 2-bromophenyl, 2-methoxyphenyl, 4-dimethylaminophenyl, 4-diethylaminophenyl, 3-bromophenyl, 4-bromophenyl, 2,3-dimethylphenyl, 4-cyclohexylphenyl, 4-pyrrolidin-1-ylphenyl, 4-piperidin-1-ylphenyl, 4-morpholin-1-ylphenyl, 2,5-difluorophenyl, 2,4-difluorophenyl, 3,4-difluorophenyl, phenethyl, benzyl, 4-methylbenzyl, 3-methylbenzyl, 4-chlorobenzyl, 3-chlorobenzyl, 2-chlorobenzyl, 4-phenoxyphenethyl, 4-methylphenethyl, 2-methylphenethyl, 4-chlorophenethyl, 2,4,6-trifluorophenyl, 2,3,5-trifluorophenyl, 2,2-difluoro-benzo[1,3]dioxol-5-yl, naphthyl, pyridin-2-yl, 6-bromo-pyridin-2-yl, 6-methyl-pyridin-2-yl, pyridin-3-yl, pyridin-4-yl, 6-methoxy-pyridin-3-yl, 6-chloro-pyridin-3-yl, 5-bromo-pyridin-3-yl, 6-bromo-pyridin-3-yl, 6-bromo-pyridin-3-yl, 6-p-tolyloxy-pyridin-3-yl, 6-(3-methoxy-phenyl)-pyridin-3-yl, 6-phenoxy-pyridin-3-yl, 6-morpholin-4-yl-pyridin-3-yl, 3,4,5,6-tetrahydro-2H-[1,2′]bipyridinyl-5′-yl, 6-furan-2-yl-pyridin-3-yl, 6-thiophen-2-yl-pyridin-3-yl, 6-thiophen-3-yl-pyridin-3-yl, 6-(3-cyanophenyl)-pyridin-3-yl, pyridinylethyl, 3-quinolinyl, 2-quinolinyl, 4-quinolinyl, 2-chloro-quinolin-3-yl, 2-chloro-6-methyl-quinolin-3-yl, 2-chloro-8-methyl-quinolin-3-yl, 2-chloro-6-methoxy-quinolin-3-yl, [1,8]naphthyridin-2-yl, quinoxalin-2-yl, 3-methyl-isoxazol-5-ylmethyl, 2-furanyl, cyclopentyl, cyclohexyl, cyclopentylmethyl, cyclohexylmethyl, cyclopentylethyl, cyclohexylethyl, phenoxymethyl, 4-chlorophenoxymethyl, benzyloxymethyl, benzyloxyethyl, ethyl, propyl, isopropyl, 2,2-dimethylpropyl, butyl, isobutyl, sec-butyl, tert-butyl, hexyl, octyl, 3-methylbutyl, methoxyethyl, or ethoxyethyl.

16. A compound, pharmaceutically acceptable salt, pharmaceutically acceptable prodrug, or pharmaceutically active metabolite as defined in claim 8, wherein Z is —C(O)— or —CH₂—.

17. A compound, pharmaceutically acceptable salt, pharmaceutically acceptable prodrug, or pharmaceutically active metabolite as defined in claim 8, wherein R³ is phenyl, 3-phenoxyphenyl, 4-phenoxyphenyl, 4-fluorophenyl, 3-fluorophenyl, 4-chlorophenyl, 3-chlorophenyl, benzo[1,3]dioxolyl, 4-methylphenyl, 3-methylphenyl, 4-t-butoxyphenyl, 2-methylphenyl, 2,3-difluorophenyl, 4-isobutylphenyl, 4-t-butylphenyl, 3,4-dibromophenyl, 3,4-dichlorophenyl, 3-chloro-4-fluorophenyl, 4-methoxyphenyl, 3-methoxyphenyl, 4-isopropylphenyl, 4-isopropoxyphenyl, 4-ethylphenyl, 3-biphenyl, 4-biphenyl, 2-chlorophenyl, 2-bromophenyl, 2-methoxyphenyl, 4-dimethylaminophenyl, 4-diethylaminophenyl, 3-bromophenyl, 4-bromophenyl, 2,3-dimethylphenyl, 4-cyclohexylphenyl, 4-pyrrolidin-1-ylphenyl, 4-piperidin-1-ylphenyl, 4-morpholin-1-ylphenyl, 2,5-difluorophenyl, 2,4-difluorophenyl, 3,4-difluorophenyl, phenethyl, benzyl, 4-methylbenzyl, 3-methylbenzyl, 4-chlorobenzyl, 3-chlorobenzyl, 2-chlorobenzyl, 4-phenoxyphenethyl, 4-methylphenethyl, 2-methylphenethyl, 4-chlorophenethyl, 2,4,6-trifluorophenyl, 2,3,5-trifluorophenyl, 2,2-difluoro-benzo[1,3]dioxol-5-yl, naphthyl, pyridin-2-yl, 6-bromo-pyridin-2-yl, 6-methyl-pyridin-2-yl, pyridin-3-yl, pyridin-4-yl, 6-methoxy-pyridin-3-yl, 6-chloro-pyridin-3-yl, 5-bromo-pyridin-3-yl, 6-bromo-pyridin-3-yl, 6-bromo-pyridin-3-yl, 6-p-tolyloxy-pyridin-3-yl, 6-(3-methoxy-phenyl)-pyridin-3-yl, 6-phenoxy-pyridin-3-yl, 6-morpholin-4-yl-pyridin-3-yl, 3,4,5,6-tetrahydro-2H-[1,2′]bipyridinyl-5′-yl, 6-furan-2-yl-pyridin-3-yl, 6-thiophen-2-yl-pyridin-3-yl, 6-thiophen-3-yl-pyridin-3-yl, 6-(3-cyanophenyl)-pyridin-3-yl, pyridinylethyl, 3-quinolinyl, 2-quinolinyl, 4-quinolinyl, 2-chloro-quinolin-3-yl, 2-chloro-6-methyl-quinolin-3-yl, 2-chloro-8-methyl-quinolin-3-yl, 2-chloro-6-methoxy-quinolin-3-yl, [1,8]naphthyridin-2-yl, quinoxalin-2-yl, 3-methyl-isoxazol-5-ylmethyl, 2-furanyl, cyclopentyl, cyclohexyl, cyclopentylmethyl, cyclohexylmethyl, cyclopentylethyl, cyclohexylethyl, phenoxymethyl, 4-chlorophenoxymethyl, benzyloxymethyl, benzyloxyethyl, ethyl, propyl, isopropyl, 2,2-dimethylpropyl, butyl, isobutyl, sec-butyl, tert-butyl, hexyl, octyl, 3-methylbutyl, methoxyethyl, or ethoxyethyl.

18. A compound, pharmaceutically acceptable salt, pharmaceutically acceptable prodrug, or pharmaceutically active metabolite as defined in claim 16, wherein R³ is phenyl, 3-phenoxyphenyl, 4-phenoxyphenyl, 4-fluorophenyl, 3-fluorophenyl, 4-chlorophenyl, 3-chlorophenyl, benzo[1,3]dioxolyl, 4-methylphenyl, 3-methylphenyl, 4-t-butoxyphenyl, 2-methylphenyl, 2,3-difluorophenyl, 4-isobutylphenyl, 4-t-butylphenyl, 3,4-dibromophenyl, 3,4-dichlorophenyl, 3-chloro-4-fluorophenyl, 4-methoxyphenyl, 3-methoxyphenyl, 4-isopropylphenyl, 4-isopropoxyphenyl, 4-ethylphenyl, 3-biphenyl, 4-biphenyl, 2-chlorophenyl, 2-bromophenyl, 2-methoxyphenyl, 4-dimethylaminophenyl, 4-diethylaminophenyl, 3-bromophenyl, 4-bromophenyl, 2,3-dimethylphenyl, 4-cyclohexylphenyl, 4-pyrrolidin-1-ylphenyl, 4-piperidin-1-ylphenyl, 4-morpholin-1-ylphenyl, 2,5-difluorophenyl, 2,4-difluorophenyl, 3,4-difluorophenyl, phenethyl, benzyl, 4-methylbenzyl, 3-methylbenzyl, 4-chlorobenzyl, 3-chlorobenzyl, 2-chlorobenzyl, 4-phenoxyphenethyl, 4-methylphenethyl, 2-methylphenethyl, 4-chlorophenethyl, 2,4,6-trifluorophenyl, 2,3,5-trifluorophenyl, 2,2-difluoro-benzo[1,3]dioxol-5-yl, naphthyl, pyridin-2-yl, 6-bromo-pyridin-2-yl, 6-methyl-pyridin-2-yl, pyridin-3-yl, pyridin-4-yl, 6-methoxy-pyridin-3-yl, 6-chloro-pyridin-3-yl, 5-bromo-pyridin-3-yl, 6-bromo-pyridin-3-yl, 6-bromo-pyridin-3-yl, 6-p-tolyloxy-pyridin-3-yl, 6-(3-methoxy-phenyl)-pyridin-3-yl, 6-phenoxy-pyridin-3-yl, 6-morpholin-4-yl-pyridin-3-yl, 3,4,5,6-tetrahydro-2H-[1,2′]bipyridinyl-5′-yl, 6-furan-2-yl-pyridin-3-yl, 6-thiophen-2-yl-pyridin-3-yl, 6-thiophen-3-yl-pyridin-3-yl, 6-(3-cyanophenyl)-pyridin-3-yl, pyridinylethyl, 3-quinolinyl, 2-quinolinyl, 4-quinolinyl, 2-chloro-quinolin-3-yl, 2-chloro-6-methyl-quinolin-3-yl, 2-chloro-8-methyl-quinolin-3-yl, 2-chloro-6-methoxy-quinolin-3-yl, [1,8]naphthyridin-2-yl, quinoxalin-2-yl, 3-methyl-isoxazol-5-ylmethyl, 2-furanyl, cyclopentyl, cyclohexyl, cyclopentylmethyl, cyclohexylmethyl, cyclopentylethyl, cyclohexylethyl, phenoxymethyl, 4-chlorophenoxymethyl, benzyloxymethyl, benzyloxyethyl, ethyl, propyl, isopropyl, 2,2-dimethylpropyl, butyl, isobutyl, sec-butyl, tert-butyl, hexyl, octyl, 3-methylbutyl, methoxyethyl, or ethoxyethyl.

19. A compound selected from the group consisting of:
4-(4-Oxazol-2-yl-4-oxo-butyl)-piperidine-1-carboxylic acid tert-butyl ester;
1-Oxazol-2-yl-4-[1-(3-phenoxy-benzyl)-piperidin-4-yl]-butan-1-one;
4-(1-Benzyl-piperidin-4-yl)-1-oxazol-2-yl-butan-1-one;
1-Oxazol-2-yl-4-(1-pyridin-2-ylmethyl-piperidin-4-yl)-butan-1-one;
1-Oxazol-2-yl-4-(1-pyridin-3-ylmethyl-piperidin-4-yl)-butan-1-one;
1-Oxazol-2-yl-4-(1-pyridin-4-ylmethyl-piperidin-4-yl)-butan-1-one;
4-[1-(4-Fluoro-benzyl)-piperidin-4-yl]-1-oxazol-2-yl-butan-1-one;
4-[1-(3-Fluoro-benzyl)-piperidin-4-yl]-1-oxazol-2-yl-butan-1-one;
4-[1-(4-Chloro-benzyl)-piperidin-4-yl]-1-oxazol-2-yl-butan-1-one;
4-[1-(3-Chloro-benzyl)-piperidin-4-yl]-1-oxazol-2-yl-butan-1-one;
4-[1-(3,4-Dibromo-benzyl)-piperidin-4-yl]-1-oxazol-2-yl-butan-1-one;
4-[1-(3,4-Dichloro-benzyl)-piperidin-4-yl]-1-oxazol-2-yl-butan-1-one;
4-[1-(3-Chloro-4-fluoro-benzyl)-piperidin-4-yl]-1-oxazol-2-yl-butan-1-one;
4-(1-Benzo[1,3]dioxol-5-ylmethyl-piperidin-4-yl)-1-oxazol-2-yl-butan-1-one;
1-Oxazol-2-yl-4-[1-(4-phenoxy-benzyl)-piperidin-4-yl]-butan-1-one;
4-[1-(4-Methoxy-benzyl)-piperidin-4-yl]-1-oxazol-2-yl-butan-1-one;
4-[1-(3-Methoxy-benzyl)-piperidin-4-yl]-1-oxazol-2-yl-butan-1-one;
4-[1-(4-Methyl-benzyl)-piperidin-4-yl]-1-oxazol-2-yl-butan-1-one;
4-[1-(3-Methyl-benzyl)-piperidin-4-yl]-1-oxazol-2-yl-butan-1-one;
4-(1-Naphthalen-2-ylmethyl-piperidin-4-yl)-1-oxazol-2-yl-butan-1-one;
1-Oxazol-2-yl-4-(1-quinolin-3-ylmethyl-piperidin-4-yl)-butan-1-one;
4-[1-(4-Isopropyl-benzyl)-piperidin-4-yl]-1-oxazol-2-yl-butan-1-one;
4-[1-(4-Isopropoxy-benzyl)-piperidin-4-yl]-1-oxazol-2-yl-butan-1-one;
4-[1-(4-tert-Butoxy-benzyl)-piperidin-4-yl]-1-oxazol-2-yl-butan-1-one;
3,3-Dimethyl-1-[4-(4-oxazol-2-yl-4-oxo-butyl)-piperidin-1-yl]-butan-1-one;
3-Methyl-1-[4-(4-oxazol-2-yl-4-oxo-butyl)-piperidin-1-yl]-butan-1-one;
1-Oxazol-2-yl-4-(1-phenylacetyl-piperidin-4-yl)-butan-1-one;
4-(1-Benzoyl-piperidin-4-yl)-1-oxazol-2-yl-butan-1-one;
4-(1-Cyclohexanecarbonyl-piperidin-4-yl)-1-oxazol-2-yl-butan-1-one;
4-(1-Isobutyryl-piperidin-4-yl)-1-oxazol-2-yl-butan-1-one;
4-(1-Cyclopentanecarbonyl-piperidin-4-yl)-1-oxazol-2-yl-butan-1-one;
4-[1-(3-Cyclopentyl-propionyl)-piperidin-4-yl]-1-oxazol-2-yl-butan-1-one;
1-Oxazol-2-yl-4-[1-(2-phenoxy-acetyl)-piperidin-4-yl]-butan-1-one;
4-[1-(2-Benzyloxy-acetyl)-piperidin-4-yl]-1-oxazol-2-yl-butan-1-one;
4-{1-[2-(4-Chloro-phenoxy)-acetyl]-piperidin-4-yl}-1-oxazol-2-yl-butan-1-one;
1-Oxazol-2-yl-4-[1-(3-phenyl-propionyl)-piperidin-4-yl]-butan-1-one;
1-Oxazol-2-yl-4-{1-[3-(4-phenoxy-phenyl)-propionyl]-piperidin-4-yl}-butan-1-one;
1-Oxazol-2-yl-4-[1-(2-p-tolyl-acetyl)-piperidin-4-yl]-butan-1-one;
1-Oxazol-2-yl-4-[1-(2-m-tolyl-acetyl)-piperidin-4-yl]-butan-1-one;
4-{1-[2-(4-Chloro-phenyl)-acetyl]-piperidin-4-yl}-1-oxazol-2-yl-butan-1-one;
4-{1-[2-(3-Chloro-phenyl)-acetyl]-piperidin-4-yl}-1-oxazol-2-yl-butan-1-one;
4-{1-[2-(2-Chloro-phenyl)-acetyl]-piperidin-4-yl}-1-oxazol-2-yl-butan-1-one;
4-{1-[2-(3-Methyl-isoxazol-5-yl)-acetyl]-piperidin-4-yl}-1-oxazol-2-yl-butan-1-one;
1-Oxazol-2-yl-4-[1-(3-p-tolyl-propionyl)-piperidin-4-yl]-butan-1-one;
1-Oxazol-2-yl-4-[1-(3-o-tolyl-propionyl)-piperidin-4-yl]-butan-1-one;
4-{1-[3-(4-Chloro-phenyl)-propionyl]-piperidin-4-yl}-1-oxazol-2-yl-butan-1-one;
1-Oxazol-2-yl-4-[1-(3-pyridin-3-yl-propionyl)-piperidin-4-yl]-butan-1-one;
4-[1-(2-Cyclopentyl-acetyl)-piperidin-4-yl]-1-oxazol-2-yl-butan-1-one;
4-[1-(2-Cyclohexyl-acetyl)-piperidin-4-yl]-1-oxazol-2-yl-butan-1-one;
4-[1-(3-Cyclohexyl-propionyl)-piperidin-4-yl]-1-oxazol-2-yl-butan-1-one;
4-Methyl-1-[4-(4-oxazol-2-yl-4-oxo-butyl)-piperidin-1-yl]-pentan-1-one;
1-Oxazol-2-yl-4-[1-(toluene-4-sulfonyl)-piperidin-4-yl]-butan-1-one;
1-Oxazol-2-yl-4-(1-phenylmethanesulfonyl-piperidin-4-yl)-butan-1-one;
1-Oxazol-2-yl-4-[1-(2-phenyl-ethanesulfonyl)-piperidin-4-yl]-butan-1-one;
4-[1-(4-Fluoro-benzenesulfonyl)-piperidin-4-yl]-1-oxazol-2-yl-butan-1-one;
1-Oxazol-2-yl-4-[1-(propane-2-sulfonyl)-piperidin-4-yl]-butan-1-one;
1-Oxazol-2-yl-4-[1-(propane-1-sulfonyl)-piperidin-4-yl]-butan-1-one;
4-[1-(Butane-1-sulfonyl)-piperidin-4-yl]-1-oxazol-2-yl-butan-1-one;
4-(1-Benzenesulfonyl-piperidin-4-yl)-1-oxazol-2-yl-butan-1-one;
4-[1-(4-Chloro-benzenesulfonyl)-piperidin-4-yl]-1-oxazol-2-yl-butan-1-one;
4-[1-(4-Methoxy-benzenesulfonyl)-piperidin-4-yl]-1-oxazol-2-yl-butan-1-one;
4-[1-(3,4-Dichloro-benzenesulfonyl)-piperidin-4-yl]-1-oxazol-2-yl-butan-1-one;
4-(4-Oxazol-2-yl-4-oxo-butyl)-piperidine-1-carboxylic acid benzyl ester;
4-(4-Oxazol-2-yl-4-oxo-butyl)-piperidine-1-carboxylic acid ethyl ester;
4-(4-Oxazol-2-yl-4-oxo-butyl)-piperidine-1-carboxylic acid 2-methoxy-ethyl ester;
4-(4-Oxazol-2-yl-4-oxo-butyl)-piperidine-1-carboxylic acid 2-benzyloxy-ethyl ester;
4-(4-Oxazol-2-yl-4-oxo-butyl)-piperidine-1-carboxylic acid 2,2-dimethyl-propyl ester;
4-(4-Oxazol-2-yl-4-oxo-butyl)-piperidine-1-carboxylic acid isobutyl ester;
4-(4-Oxazol-2-yl-4-oxo-butyl)-piperidine-1-carboxylic acid isopropyl ester;
4-(4-Oxazol-2-yl-4-oxo-butyl)-piperidine-1-carboxylic acid propyl ester;
4-[1-(4-Ethyl-benzyl)-piperidin-4-yl]-1-oxazol-2-yl-butan-1-one;
4-(1-Biphenyl-3-ylmethyl-piperidin-4-yl)-1-oxazol-2-yl-butan-1-one;
4-(1-Biphenyl-4-ylmethyl-piperidin-4-yl)-1-oxazol-2-yl-butan-1-one;
4-[1-(6-Methoxy-pyridin-3-ylmethyl)-piperidin-4-yl]-1-oxazol-2-yl-butan-1-one;
4-[1-(6-Chloro-pyridin-3-ylmethyl)-piperidin-4-yl]-1-oxazol-2-yl-butan-1-one;
4-[1-(6-Bromo-pyridin-3-ylmethyl)-piperidin-4-yl]-1-oxazol-2-yl-butan-1-one;
4-[1-(6-Bromo-pyridin-2-ylmethyl)-piperidin-4-yl]-1-oxazol-2-yl-butan-1-one;
4-[1-(5-Bromo-pyridin-3-ylmethyl)-piperidin-4-yl]-1-oxazol-2-yl-butan-1-one;
4-[1-(6-Methyl-pyridin-2-ylmethyl)-piperidin-4-yl]-1-oxazol-2-yl-butan-1-one;
4-[1-(2-Methyl-benzyl)-piperidin-4-yl]-1-oxazol-2-yl-butan-1-one;
4-[1-(2,3-Difluoro-benzyl)-piperidin-4-yl]-1-oxazol-2-yl-butan-1-one;
4-[1-(4-Isobutyl-benzyl)-piperidin-4-yl]-1-oxazol-2-yl-butan-1-one;
4-[1-(4-tert-Butyl-benzyl)-piperidin-4-yl]-1-oxazol-2-yl-butan-1-one;
4-[1-(2-Chloro-benzyl)-piperidin-4-yl]-1-oxazol-2-yl-butan-1-one;
4-[1-(2-Bromo-benzyl)-piperidin-4-yl]-1-oxazol-2-yl-butan-1-one;
4-(1-Cyclohexylmethyl-piperidin-4-yl)-1-oxazol-2-yl-butan-1-one;
4-[1-(2-Methoxy-benzyl)-piperidin-4-yl]-1-oxazol-2-yl-butan-1-one;
4-[1-(4-Dimethylamino-benzyl)-piperidin-4-yl]-1-oxazol-2-yl-butan-1-one;
4-[1-(4-Diethylamino-benzyl)-piperidin-4-yl]-1-oxazol-2-yl-butan-1-one;
4-[1-(3-Bromo-benzyl)-piperidin-4-yl]-1-oxazol-2-yl-butan-1-one;
4-[1-(4-Bromo-benzyl)-piperidin-4-yl]-1-oxazol-2-yl-butan-1-one;
1-Oxazol-2-yl-4-(1-quinolin-2-ylmethyl-piperidin-4-yl)-butan-1-one;
1-Oxazol-2-yl-4-(1-quinolin-4-ylmethyl-piperidin-4-yl)-butan-1-one;
4-[1-(2,3-Dimethyl-benzyl)-piperidin-4-yl]-1-oxazol-2-yl-butan-1-one;
1-Oxazol-2-yl-4-(1-phenethyl-piperidin-4-yl)-butan-1-one;
1-Oxazol-2-yl-4-[1-(6-p-tolyloxy-pyridin-3-ylmethyl)-piperidin-4-yl]-butan-1-one;
4-[1-(2-Chloro-quinolin-3-ylmethyl)-piperidin-4-yl]-1-oxazol-2-yl-butan-1-one;
4-[1-(2-Chloro-6-methyl-quinolin-3-ylmethyl)-piperidin-4-yl]-1-oxazol-2-yl-butan-1-one;
4-[1-(2-Chloro-8-methyl-quinolin-3-ylmethyl)-piperidin-4-yl]-1-oxazol-2-yl-butan-1-one;
4-[1-(2-Chloro-6-methoxy-quinolin-3-ylmethyl)-piperidin-4-yl]-1-oxazol-2-yl-butan-1-one;
4-[1-(4-Cyclohexyl-benzyl)-piperidin-4-yl]-1-oxazol-2-yl-butan-1-one;
1-Oxazol-2-yl-4-[1-(4-pyrrolidin-1-yl-benzyl)-piperidin-4-yl]-butan-1-one;
1-Oxazol-2-yl-4-[1-(4-piperidin-1-yl-benzyl)-piperidin-4-yl]-butan-1-one;
4-({-[6-(3-Methoxy-phenyl)-pyridin-3-ylmethyl]-piperidin-4-yl}-1-oxazol-2-yl-butan-1-one;
1-Oxazol-2-yl-4-[1-(6-phenoxy-pyridin-3-ylmethyl)-piperidin-4-yl]-butan-1-one;
4-[1-(4-Morpholin-4-yl-benzyl)-piperidin-4-yl]-1-oxazol-2-yl-butan-1-one;
4-[1-(6-Morpholin-4-yl-pyridin-3-ylmethyl)-piperidin-4-yl]-1-oxazol-2-yl-butan-1-one;
1-Oxazol-2-yl-4-[1-(3,4,5,6-tetrahydro-2H-[1,2]bipyridinyl-5′-ylmethyl)-piperidin-4-yl]-butan-1-one;
4-[1-(6-Furan-2-yl-pyridin-3-ylmethyl)-piperidin-4-yl]-1-oxazol-2-yl-butan-1-one;
1-Oxazol-2-yl-4-[1-(6-thiophen-2-yl-pyridin-3-ylmethyl)-piperidin-4-yl]-butan-1-one;
1-Oxazol-2-yl-4-[1-(6-thiophen-3-yl-pyridin-3-ylmethyl)-piperidin-4-yl]-butan-1-one;
3-{5-[4-(4-Oxazol-2-yl-4-oxo-butyl)-piperidin-1-ylmethyl]-pyridin-2-yl}-benzonitrile;
4-[1-(2,5-Difluoro-benzyl)-piperidin-4-yl]-1-oxazol-2-yl-butan-1-one;
4-[1-(2,4-Difluoro-benzyl)-piperidin-4-yl]-1-oxazol-2-yl-butan-1-one;
4-[1-(3,4-Difluoro-benzyl)-piperidin-4-yl]-1-oxazol-2-yl-butan-1-one;
4-(1-[1,8]Naphthyridin-2-ylmethyl-piperidin-4-yl)-1-oxazol-2-yl-butan-1-one;
1-Oxazol-2-yl-4-(1-quinoxalin-2-ylmethyl-piperidin-4-yl)-butan-1-one;
4-(1-Furan-2-ylmethyl-piperidin-4-yl)-1-oxazol-2-yl-butan-1-one;
4-[4-Oxo-4-(5-pyridin-2-yl-oxazol-2-yl)-butyl]-piperidine-1-carboxylic acid tert-butyl ester;
4-[4-(5-Furan-2-yl-oxazol-2-yl)-4-oxo-butyl]-piperidine-1-carboxylic acid tert-butyl ester;
4-(1-Benzyl-piperidin-4-yl)-1-(5-furan-2-yl-oxazol-2-yl)-butan-1-one;
1-Oxazol-2-yl-4-[1-(2,4,6-trifluoro-benzyl)-piperidin-4-yl]-butan-1-one;
1-Oxazol-2-yl-4-[1-(2,3,5-trifluoro-benzyl)-piperidin-4-yl]-butan-1-one;
4-[1-(2,2-Difluoro-benzo[1,3]dioxol-5-ylmethyl)-piperidin-4-yl]-1-oxazol-2-yl-butan-1-one;
4-(1-Heptyl-piperidin-4-yl)-1-oxazol-2-yl-butan-1-one;
4-(1-Nonyl-piperidin-4-yl)-1-oxazol-2-yl-butan-1-one;
4-[1-(3-Methyl-butyl)-piperidin-4-yl]-1-oxazol-2-yl-butan-1-one; and
1-Oxazol-2-yl-4-(1-pentyl-piperidin-4-yl)-butan-1-one;
and pharmaceutically acceptable salts thereof.

20. A compound selected from the group consisting of:
4-[4-(5-Carboxy-oxazol-2-yl)-4-oxo-butyl]-piperidine-1-carboxylic acid tert-butyl ester;
1-Oxazol-2-yl-4-[1-(3,4,5-trifluoro-benzyl)-piperidin-4-yl]-butan-1-one;
4-[1-(6-Isopropyl-pyridin-3-ylmethyl)-piperidin-4-yl]-1-oxazol-2-yl-butan-1-one;
4-[1-(4-Chloro-3-trifluoromethyl-benzyl)-piperidin-4-yl]-1-oxazol-2-yl-butan-1-one;
4-[1-(4-Cyclohexyloxy-benzyl)-piperidin-4-yl]-1-oxazol-2-yl-butan-1-one;
4-[1-(3-Isopropoxy-benzyl)-piperidin-4-yl]-1-oxazol-2-yl-butan-1-one;
4-[1-(3-Cyclohexyloxy-benzyl)-piperidin-4-yl]-1-oxazol-2-yl-butan-1-one;
4-[1-(3-Fluoro-4-trifluoromethyl-benzyl)-piperidin-4-yl]-1-oxazol-2-yl-butan-1-one;
4-[1-(2-Fluoro-biphenyl-4-ylmethyl)-piperidin-4-yl]-1-oxazol-2-yl-butan-1-one;
4-[1-(6-tert-Butyl-pyridin-3-ylmethyl)-piperidin-4-yl]-1-oxazol-2-yl-butan-1-one;
4-[1-(3-Isopropyl-benzyl)-piperidin-4-yl]-1-oxazol-2-yl-butan-1-one;
4-[1-(4-Imidazol-1-yl-benzyl)-piperidin-4-yl]-1-oxazol-2-yl-butan-1-one;
1-Oxazol-2-yl-4-[1-(1-phenyl-ethyl)-piperidin-4-yl]-butan-1-one;
2-[4-(1-Biphenyl-3-ylmethyl-piperidin-4-yl)-butyryl]-oxazole-5-carboxylic acid;
2-{4-[1-(4-Isopropyl-benzyl)-piperidin-4-yl]-butyryl}-oxazole-5-carboxylic acid;
2-{4-[1-(3-Phenoxy-benzyl)-piperidin-4-yl]-butyryl}-oxazole-5-carboxylic acid;
2-{4-[1-(Toluene-4-sulfonyl)-piperidin-4-yl]-butyryl}-oxazole-5-carboxylic acid;
6-(2-{4-[1-(4-Isopropyl-benzyl)-piperidin-4-yl]-butyryl}-oxazol-5-yl)-pyridine-2-carboxylic acid methyl ester;
6-(2-{4-[1-(4-Isopropyl-benzyl)-piperidin-4-yl]-butyryl}-oxazol-5-yl)-pyridine-2-carboxylic acid;
6-{2-[4-(1-Biphenyl-3-ylmethyl-piperidin-4-yl)-butyryl]-oxazol-5-yl}-pyridine-2-carboxylic acid;
6-(2-{4-[1-(3-Cyclohexyloxy-benzyl)-piperidin-4-yl]-butyryl}-oxazol-5-yl)-pyridine-2-carboxylic acid; and
6-(2-{4-[1-(Toluene-4-sulfonyl)-piperidin-4-yl]-butyryl}-oxazol-5-yl)-pyridine-2-carboxylic acid;
and pharmaceutically acceptable salts thereof.

21. A compound or pharmaceutically acceptable salt according to claim 19.

22. A compound or pharmaceutically acceptable salt according to claim 20.

23. A pharmaceutical composition for treating a disease, disorder, or medical condition mediated by FAAH activity, comprising:
(a) an effective amount of an agent selected from the group consisting of compounds of Formula (I): 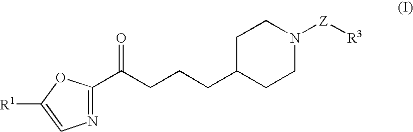
wherein:
R¹ is —H; a —CO₂C₁₋₄alkyl or —CO₂H group; or a phenyl, pyridyl, pyridazinyl, pyrimidinyl, pyrazinyl, thiophenyl, furanyl, or oxazolyl group, each unsubstituted or substituted with —CO₂H or —CO₂C₁₋₄alkyl;
Z is —C(O)—, —CO₂—, —SO₂—, —C(O)NH—, —CH₂—, or —CH(CH₃)—; and
R³ is:
(a) —(CH₂)ₙ—R⁴, where n is 0, 1, or 2, and R⁴ is:
(i) phenyl, unsubstituted or substituted with one, or two, or three R^{a} moieties or where two adjacent R^{a} moieties together form —O(CH₂)₁₋₂O— or —O(CF₂)O—;
where each R^{a} moiety is —C₁₋₇alkyl, —C₃₋₇cycloalkyl, —C₂₋₇alkenyl, —OH, —OC₁₋₇alkyl, —OC₃₋₇cycloalkyl, phenyl unsubstituted or substituted with R^{b}, phenoxy unsubstituted or substituted with R^{b}, furanyl, thiophenyl, imidazolyl, fluoro, chloro, bromo, iodo, —CF₃, —OCF₃, —SC₁₋₄alkyl, —SO₂C₁₋₄alkyl, —SOC₁₋₄alkyl, —CN, —CO₂C₁₋₄alkyl, —CO₂H, —COC₁₋₄alkyl, —SO₂NR^{c}R^{d}, —NR^{c}SO₂R^{d}, —C(O)NR^{c}R^{d}, —NR^{c}C(O)R^{d}, or N(R^{c})R^{d};
where R^{b} is selected from the group consisting of —C₁₋₄alkyl, —OC₁₋₄alkyl, fluoro, chloro, bromo, iodo, —CN, —OH, —CF₃, —OCF₃, and —NO₂; and
where R^{c} and R^{d} are each independently —H or —C₁₋₇alkyl, or R^{c} and R^{d} taken together form a 3- to 7-membered heterocycloalkyl ring;
(ii) a five- or six-membered monocyclic heteroaryl ring, unsubstituted or substituted with one or two R^{a} moieties as defined above;
(iii) naphthyl, unsubstituted or substituted one or two R^{e} moieties, where each R^{e} moiety is independently selected from the group consisting of —C₁₋₄alkyl, —OC₁₋₄alkyl, fluoro, chloro, bromo, iodo, —CN, —OH, —CF₃, —OCF₃, and —NO₂;
(iv) a nine- or ten-membered fused bicyclic heteroaryl, unsubstituted or substituted with one or two R^{e} moieties; or
(v) —C₃₋₉cycloalkyl;
(b) —(CH₂)ₓO(CH₂)_{y}R⁴, where when Z is —C(O)—, —SO₂—, or —CH₂—, x is 1 or 2 and y is 0, 1, or 2, and when Z is —CO₂— or —C(O)NH—, x is 2 and y is 0, 1, or 2;
(c) —C₂₋₉alkyl; or
(d) —C₂₋₉alkyl, where one carbon chain member is replaced by nitrogen or oxygen;
and pharmaceutically acceptable salts, pharmaceutically acceptable prodrugs, and pharmaceutically active metabolites thereof; and
(b) a pharmaceutically acceptable excipient.

24. A pharmaceutical composition according to claim 23, wherein said agent is selected from the group consisting of:
4-[4-(5-Carboxy-oxazol-2-yl)-4-oxo-butyl]-piperidine-1-carboxylic acid tert-butyl ester;
1-Oxazol-2-yl-4-[1-(3,4,5-trifluoro-benzyl)-piperidin-4-yl]-butan-1-one;
4-[1-(6-Isopropyl-pyridin-3-ylmethyl)-piperidin-4-yl]-1-oxazol-2-yl-butan-1-one;
4-[1-(4-Chloro-3-trifluoromethyl-benzyl)-piperidin-4-yl]-1-oxazol-2-yl-butan-1-one;
4-[1-(4-Cyclohexyloxy-benzyl)-piperidin-4-yl]-1-oxazol-2-yl-butan-1-one;
4-[1-(3-Isopropoxy-benzyl)-piperidin-4-yl]-1-oxazol-2-yl-butan-1-one;
4-[1-(3-Cyclohexyloxy-benzyl)-piperidin-4-yl]-1-oxazol-2-yl-butan-1-one;
4-[1-(3-Fluoro-4-trifluoromethyl-benzyl)-piperidin-4-yl]-1-oxazol-2-yl-butan-1-one;
4-[1-(2-Fluoro-biphenyl-4-ylmethyl)-piperidin-4-yl]-1-oxazol-2-yl-butan-1-one;
4-[1-(6-tert-Butyl-pyridin-3-ylmethyl)-piperidin-4-yl]-1-oxazol-2-yl-butan-1-one;
4-[1-(3-Isopropyl-benzyl)-piperidin-4-yl]-1-oxazol-2-yl-butan-1-one;
4-[1-(4-Imidazol-1-yl-benzyl)-piperidin-4-yl]-1-oxazol-2-yl-butan-1-one;
1-Oxazol-2-yl-4-[1-(1-phenyl-ethyl)-piperidin-4-yl]-butan-1-one;
2-[4-(1-Biphenyl-3-ylmethyl-piperidin-4-yl)-butyryl]-oxazole-5-carboxylic acid;
2-{4-[1-(4-Isopropyl-benzyl)-piperidin-4-yl]-butyryl}-oxazole-5-carboxylic acid;
2-{4-[1-(3-Phenoxy-benzyl)-piperidin-4-yl]-butyryl}-oxazole-5-carboxylic acid;
2-{4-[1-(Toluene-4-sulfonyl)-piperidin-4-yl]-butyryl}-oxazole-5-carboxylic acid;
6-(2-{4-[1-(4-Isopropyl-benzyl)-piperidin-4-yl]-butyryl}-oxazol-5-yl)-pyridine-2-carboxylic acid methyl ester;
6-(2-{4-[1-(4-Isopropyl-benzyl)-piperidin-4-yl]-butyryl}-oxazol-5-yl)-pyridine-2-carboxylic acid;
6-{2-[4-(1-Biphenyl-3-ylmethyl-piperidin-4-yl)-butyryl]-oxazol-5-yl}-pyridine-2-carboxylic acid;
6-(2-{4-[1-(3-Cyclohexyloxy-benzyl)-piperidin-4-yl]-butyryl}-oxazol-5-yl)-pyridine-2-carboxylic acid; and
6-(2-{4-[1-(Toluene-4-sulfonyl)-piperidin-4-yl]-butyryl}-oxazol-5-yl)-pyridine-2-carboxylic acid;
and pharmaceutically acceptable salts thereof.

25. A pharmaceutical composition according to claim 23, further comprising: an analgesic selected from the group consisting of opioids and non-steroidal anti-inflammatory drugs.

26. A pharmaceutical composition according to claim 23, further comprising: an additional active ingredient selected from the group consisting of aspirin, acetaminophen, opioids, ibuprofen, naproxen, COX-2 inhibitors, gabapentin, pregabalin, and tramadol.

27. A pharmaceutical composition for treating a disease, disorder, or medical condition mediated by FAAH activity, comprising:
(a) an effective amount of an agent selected from the group consisting of compounds of Formula (IA): 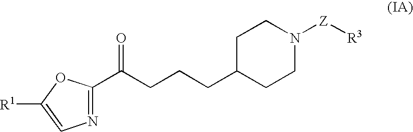
wherein:
R¹ is —H, or a phenyl, pyridyl, pyridazinyl, pyrimidinyl, pyrazinyl, thiophenyl, furanyl, or oxazolyl group;
Z is —C(O)—, —CO₂—, —SO₂—, —C(O)NH—, or —CH₂—; and
R³ is:
(a) —(CH₂)ₙ—R⁴, where n is 0, 1, or 2, and R⁴ is:
(i) phenyl, unsubstituted or substituted with one, or two, or three R^{a} moieties or where two adjacent R^{a} moieties together form —O(CH₂)₁₋₂O— or —O(CF₂)O—;
where each R^{a} moiety is —C₁₋₇alkyl, —C₃₋₇cycloalkyl, —C₂₋₇alkenyl, —OH, —OC₁₋₇alkyl, phenyl unsubstituted or substituted with R^{b}, phenoxy unsubstituted or substituted with R^{b}, furanyl, thiophenyl, fluoro, chloro, bromo, iodo, —CF₃, —OCF₃, —SC₁₋₄alkyl, —SO₂C₁₋₄alkyl, —SOC₁₋₄alkyl, —CN, —CO₂C₁₋₄alkyl, —CO₂H, —COC₁₋₄alkyl, —SO₂NR^{c}R^{d}, —NR^{c}SO₂R^{d}, —C(O)NR^{c}R^{d}, —NR^{c}C(O)R^{d}, or —N(R^{c})R^{d};
where R^{b} is selected from the group consisting of —C₁₋₄alkyl, —OC₁₋₄alkyl, fluoro, chloro, bromo, iodo, —CN, —OH, —CF₃, —OCF₃, and —NO₂; and
where R^{c} and R^{d} are each independently —H or —C₁₋₇alkyl, or R^{c} and R^{d} taken together form a 3- to 7-membered heterocycloalkyl ring;
(ii) a five- or six-membered monocyclic heteroaryl ring, unsubstituted or substituted with one or two R^{a} moieties as defined above;
(iii) naphthyl, unsubstituted or substituted one or two R^{e} moieties, where each R^{e} moiety is independently selected from the group consisting of —C₁₋₄alkyl, —OC₁₋₄alkyl, fluoro, chloro, bromo, iodo, —CN, —OH, —CF₃, —OCF₃, and —NO₂;
(iv) a nine- or ten-membered fused bicyclic heteroaryl, unsubstituted or substituted with one or two R^{e} moieties; or
(v) —C₃₋₉cycloalkyl;
(b) —(CH₂)ₓO(CH₂)_{y}R⁴, where when Z is —C(O)—, —SO₂—, or —CH₂—, x is 1 or 2 and y is 0, 1, or 2, and when Z is —CO₂— or —C(O)NH—, x is 2 and y is 0, 1, or 2;
(c) —C₂₋₉alkyl; or
(d) —C₂₋₉alkyl, where one carbon chain member is replaced by nitrogen or oxygen;
and pharmaceutically acceptable salts, pharmaceutically acceptable prodrugs, and pharmaceutically active metabolites thereof; and
(b) a pharmaceutically acceptable excipient.

28. A pharmaceutical composition according to claim 27, wherein said agent is selected from the group consisting of:
4-(4-Oxazol-2-yl-4-oxo-butyl)-piperidine-1-carboxylic acid tert-butyl ester;
1-Oxazol-2-yl-4-[1-(3-phenoxy-benzyl)-piperidin-4-yl]-butan-1-one;
4-(1-Benzyl-piperidin-4-yl)-1-oxazol-2-yl-butan-1-one;
1-Oxazol-2-yl-4-(1-pyridin-2-ylmethyl-piperidin-4-yl)-butan-1-one;
1-Oxazol-2-yl-4-(1-pyridin-3-ylmethyl-piperidin-4-yl)-butan-1-one;
1-Oxazol-2-yl-4-(1-pyridin-4-ylmethyl-piperidin-4-yl)-butan-1-one;
4-[1-(4-Fluoro-benzyl)-piperidin-4-yl]-1-oxazol-2-yl-butan-1-one;
4-[1-(3-Fluoro-benzyl)-piperidin-4-yl]-1-oxazol-2-yl-butan-1-one;
4-[1-(4-Chloro-benzyl)-piperidin-4-yl]-1-oxazol-2-yl-butan-1-one;
4-[1-(3-Chloro-benzyl)-piperidin-4-yl]-1-oxazol-2-yl-butan-1-one;
4-[1-(3,4-Dibromo-benzyl)-piperidin-4-yl]-1-oxazol-2-yl-butan-1-one;
4-[1-(3,4-Dichloro-benzyl)-piperidin-4-yl]-1-oxazol-2-yl-butan-1-one;
4-[1-(3-Chloro-4-fluoro-benzyl)-piperidin-4-yl]-1-oxazol-2-yl-butan-1-one;
4-(1-Benzo[1,3]dioxol-5-ylmethyl-piperidin-4-yl)-1-oxazol-2-yl-butan-1-one;
1-Oxazol-2-yl-4-[1-(4-phenoxy-benzyl)-piperidin-4-yl]-butan-1-one;
4-[1-(4-Methoxy-benzyl)-piperidin-4-yl]-1-oxazol-2-yl-butan-1-one;
4-[1-(3-Methoxy-benzyl)-piperidin-4-yl]-1-oxazol-2-yl-butan-1-one;
4-[1-(4-Methyl-benzyl)-piperidin-4-yl]-1-oxazol-2-yl-butan-1-one;
4-[1-(3-Methyl-benzyl)-piperidin-4-yl]-1-oxazol-2-yl-butan-1-one;
4-(1-Naphthalen-2-ylmethyl-piperidin-4-yl)-1-oxazol-2-yl-butan-1-one;
1-Oxazol-2-yl-4-(1-quinolin-3-ylmethyl-piperidin-4-yl)-butan-1-one;
4-[1-(4-Isopropyl-benzyl)-piperidin-4-yl]-1-oxazol-2-yl-butan-1-one;
4-[1-(4-Isopropoxy-benzyl)-piperidin-4-yl]-1-oxazol-2-yl-butan-1-one;
4-[1-(4-tert-Butoxy-benzyl)-piperidin-4-yl]-1-oxazol-2-yl-butan-1-one;
3,3-Dimethyl-1-[4-(4-oxazol-2-yl-4-oxo-butyl)-piperidin-1-yl]-butan-1-one;
3-Methyl-1-[4-(4-oxazol-2-yl-4-oxo-butyl)-piperidin-1-yl]-butan-1-one;
1-Oxazol-2-yl-4-(1-phenylacetyl-piperidin-4-yl)-butan-1-one;
4-(1-Benzoyl-piperidin-4-yl)-1-oxazol-2-yl-butan-1-one;
4-(1-Cyclohexanecarbonyl-piperidin-4-yl)-1-oxazol-2-yl-butan-1-one;
4-(1-Isobutyryl-piperidin-4-yl)-1-oxazol-2-yl-butan-1-one;
4-(1-Cyclopentanecarbonyl-piperidin-4-yl)-1-oxazol-2-yl-butan-1-one;
4-[1-(3-Cyclopentyl-propionyl)-piperidin-4-yl]-1-oxazol-2-yl-butan-1-one;
1-Oxazol-2-yl-4-[1-(2-phenoxy-acetyl)-piperidin-4-yl]-butan-1-one;
4-[1-(2-Benzyloxy-acetyl)-piperidin-4-yl]-1-oxazol-2-yl-butan-1-one;
4-{1-[2-(4-Chloro-phenoxy)-acetyl]-piperidin-4-yl}-1-oxazol-2-yl-butan-1-one;
1-Oxazol-2-yl-4-[1-(3-phenyl-propionyl)-piperidin-4-yl]-butan-1-one;
1-Oxazol-2-yl-4-{1-[3-(4-phenoxy-phenyl)-propionyl]-piperidin-4-yl}-butan-1-one;
1-Oxazol-2-yl-4-[1-(2-p-tolyl-acetyl)-piperidin-4-yl]-butan-1-one;
1-Oxazol-2-yl-4-[1-(2-m-tolyl-acetyl)-piperidin-4-yl]-butan-1-one;
4-{1-[2-(4-Chloro-phenyl)-acetyl]-piperidin-4-yl}-1-oxazol-2-yl-butan-1-one;
4-{1-[2-(3-Chloro-phenyl)-acetyl]-piperidin-4-yl}-1-oxazol-2-yl-butan-1-one;
4-({-[2-(2-Chloro-phenyl)-acetyl]-piperidin-4-yl}-1-oxazol-2-yl-butan-1-one;
4-{1-[2-(3-Methyl-isoxazol-5-yl)-acetyl]-piperidin-4-yl}-1-oxazol-2-yl-butan-1-one;
1-Oxazol-2-yl-4-[1-(3-p-tolyl-propionyl)-piperidin-4-yl]-butan-1-one;
1-Oxazol-2-yl-4-[1-(3-o-tolyl-propionyl)-piperidin-4-yl]-butan-1-one;
4-{1-[3-(4-Chloro-phenyl)-propionyl]-piperidin-4-yl}-1-oxazol-2-yl-butan-1-one;
1-Oxazol-2-yl-4-[1-(3-pyridin-3-yl-propionyl)-piperidin-4-yl]-butan-1-one;
4-[1-(2-Cyclopentyl-acetyl)-piperidin-4-yl]-1-oxazol-2-yl-butan-1-one;
4-[1-(2-Cyclohexyl-acetyl)-piperidin-4-yl]-1-oxazol-2-yl-butan-1-one;
4-[1-(3-Cyclohexyl-propionyl)-piperidin-4-yl]-1-oxazol-2-yl-butan-1-one;
4-Methyl-1-[4-(4-oxazol-2-yl-4-oxo-butyl)-piperidin-1-yl]-pentan-1-one;
1-Oxazol-2-yl-4-[1-(toluene-4-sulfonyl)-piperidin-4-yl]-butan-1-one;
1-Oxazol-2-yl-4-(1-phenylmethanesulfonyl-piperidin-4-yl)-butan-1-one;
1-Oxazol-2-yl-4-[1-(2-phenyl-ethanesulfonyl)-piperidin-4-yl]-butan-1-one;
4-[1-(4-Fluoro-benzenesulfonyl)-piperidin-4-yl]-1-oxazol-2-yl-butan-1-one;
1-Oxazol-2-yl-4-[1-(propane-2-sulfonyl)-piperidin-4-yl]-butan-1-one;
1-Oxazol-2-yl-4-[1-(propane-1-sulfonyl)-piperidin-4-yl]-butan-1-one;
4-[1-(Butane-1-sulfonyl)-piperidin-4-yl]-1-oxazol-2-yl-butan-1-one;
4-(1-Benzenesulfonyl-piperidin-4-yl)-1-oxazol-2-yl-butan-1-one;
4-[1-(4-Chloro-benzenesulfonyl)-piperidin-4-yl]-1-oxazol-2-yl-butan-1-one;
4-[1-(4-Methoxy-benzenesulfonyl)-piperidin-4-yl]-1-oxazol-2-yl-butan-1-one;
4-[1-(3,4-Dichloro-benzenesulfonyl)-piperidin-4-yl]-1-oxazol-2-yl-butan-1-one;
4-(4-Oxazol-2-yl-4-oxo-butyl)-piperidine-1-carboxylic acid benzyl ester;
4-(4-Oxazol-2-yl-4-oxo-butyl)-piperidine-1-carboxylic acid ethyl ester;
4-(4-Oxazol-2-yl-4-oxo-butyl)-piperidine-1-carboxylic acid 2-methoxy-ethyl ester;
4-(4-Oxazol-2-yl-4-oxo-butyl)-piperidine-1-carboxylic acid 2-benzyloxy-ethyl ester;
4-(4-Oxazol-2-yl-4-oxo-butyl)-piperidine-1-carboxylic acid 2,2-dimethyl-propyl ester;
4-(4-Oxazol-2-yl-4-oxo-butyl)-piperidine-1-carboxylic acid isobutyl ester;
4-(4-Oxazol-2-yl-4-oxo-butyl)-piperidine-1-carboxylic acid isopropyl ester;
4-(4-Oxazol-2-yl-4-oxo-butyl)-piperidine-1-carboxylic acid propyl ester;
4-[1-(4-Ethyl-benzyl)-piperidin-4-yl]-1-oxazol-2-yl-butan-1-one;
4-(1-Biphenyl-3-ylmethyl-piperidin-4-yl)-1-oxazol-2-yl-butan-1-one;
4-(1-Biphenyl-4-ylmethyl-piperidin-4-yl)-1-oxazol-2-yl-butan-1-one;
4-[1-(6-M ethoxy-pyridin-3-ylmethyl)-piperidin-4-yl]-1-oxazol-2-yl-butan-1-one;
4-[1-(6-Chloro-pyridin-3-ylmethyl)-piperidin-4-yl]-1-oxazol-2-yl-butan-1-one;
4-[1-(6-Bromo-pyridin-3-ylmethyl)-piperidin-4-yl]-1-oxazol-2-yl-butan-1-one;
4-[1-(6-Bromo-pyridin-2-ylmethyl)-piperidin-4-yl]-1-oxazol-2-yl-butan-1-one;
4-[1-(5-Bromo-pyridin-3-ylmethyl)-piperidin-4-yl]-1-oxazol-2-yl-butan-1-one;
4-[1-(6-Methyl-pyridin-2-ylmethyl)-piperidin-4-yl]-1-oxazol-2-yl-butan-1-one;
4-[1-(2-Methyl-benzyl)-piperidin-4-yl]-1-oxazol-2-yl-butan-1-one;
4-[1-(2,3-Difluoro-benzyl)-piperidin-4-yl]-1-oxazol-2-yl-butan-1-one;
4-[1-(4-Isobutyl-benzyl)-piperidin-4-yl]-1-oxazol-2-yl-butan-1-one;
4-[1-(4-tert-Butyl-benzyl)-piperidin-4-yl]-1-oxazol-2-yl-butan-1-one;
4-[1-(2-Chloro-benzyl)-piperidin-4-yl]-1-oxazol-2-yl-butan-1-one;
4-[1-(2-Bromo-benzyl)-piperidin-4-yl]-1-oxazol-2-yl-butan-1-one;
4-(1-Cyclohexylmethyl-piperidin-4-yl)-1-oxazol-2-yl-butan-1-one;
4-[1-(2-Methoxy-benzyl)-piperidin-4-yl]-1-oxazol-2-yl-butan-1-one;
4-[1-(4-Dimethylamino-benzyl)-piperidin-4-yl]-1-oxazol-2-yl-butan-1-one;
4-[1-(4-Diethylamino-benzyl)-piperidin-4-yl]-1-oxazol-2-yl-butan-1-one;
4-[1-(3-Bromo-benzyl)-piperidin-4-yl]-1-oxazol-2-yl-butan-1-one;
4-[1-(4-Bromo-benzyl)-piperidin-4-yl]-1-oxazol-2-yl-butan-1-one;
1-Oxazol-2-yl-4-(1-quinolin-2-ylmethyl-piperidin-4-yl)-butan-1-one;
1-Oxazol-2-yl-4-(1-quinolin-4-ylmethyl-piperidin-4-yl)-butan-1-one;
4-[1-(2,3-Dimethyl-benzyl)-piperidin-4-yl]-1-oxazol-2-yl-butan-1-one;
1-Oxazol-2-yl-4-(1-phenethyl-piperidin-4-yl)-butan-1-one;
1-Oxazol-2-yl-4-[1-(6-p-tolyloxy-pyridin-3-ylmethyl)-piperidin-4-yl]-butan-1-one;
4-[1-(2-Chloro-quinolin-3-ylmethyl)-piperidin-4-yl]-1-oxazol-2-yl-butan-1-one;
4-[1-(2-Chloro-6-methyl-quinolin-3-ylmethyl)-piperidin-4-yl]-1-oxazol-2-yl-butan-1-one;
4-[1-(2-Chloro-8-methyl-quinolin-3-ylmethyl)-piperidin-4-yl]-1-oxazol-2-yl-butan-1-one;
4-[1-(2-Chloro-6-methoxy-quinolin-3-ylmethyl)-piperidin-4-yl]-1-oxazol-2-yl-butan-1-one;
4-[1-(4-Cyclohexyl-benzyl)-piperidin-4-yl]-1-oxazol-2-yl-butan-1-one;
1-Oxazol-2-yl-4-[1-(4-pyrrolidin-1-yl-benzyl)-piperidin-4-yl]-butan-1-one;
1-Oxazol-2-yl-4-[1-(4-piperidin-1-yl-benzyl)-piperidin-4-yl]-butan-1-one;
4-{1-[6-(3-Methoxy-phenyl)-pyridin-3-ylmethyl]-piperidin-4-yl}-1-oxazol-2-yl-butan-1-one;
1-Oxazol-2-yl-4-[1-(6-phenoxy-pyridin-3-ylmethyl)-piperidin-4-yl]-butan-1-one;
4-[1-(4-Morpholin-4-yl-benzyl)-piperidin-4-yl]-1-oxazol-2-yl-butan-1-one;
4-[1-(6-Morpholin-4-yl-pyridin-3-ylmethyl)-piperidin-4-yl]-1-oxazol-2-yl-butan-1-one;
1-Oxazol-2-yl-4-[1-(3,4,5,6-tetrahydro-2H-[1,2′]bipyridinyl-5′-ylmethyl)-piperidin-4-yl]-butan-1-one;
4-[1-(6-Furan-2-yl-pyridin-3-ylmethyl)-piperidin-4-yl]-1-oxazol-2-yl-butan-1-one;
1-Oxazol-2-yl-4-[1-(6-thiophen-2-yl-pyridin-3-ylmethyl)-piperidin-4-yl]-butan-1-one;
1-Oxazol-2-yl-4-[1-(6-thiophen-3-yl-pyridin-3-ylmethyl)-piperidin-4-yl]-butan-1-one;
3-{5-[4-(4-Oxazol-2-yl-4-oxo-butyl)-piperidin-1-ylmethyl]-pyridin-2-yl}-benzonitrile;
4-[1-(2,5-Difluoro-benzyl)-piperidin-4-yl]-1-oxazol-2-yl-butan-1-one;
4-[1-(2,4-Difluoro-benzyl)-piperidin-4-yl]-1-oxazol-2-yl-butan-1-one;
4-[1-(3,4-Difluoro-benzyl)-piperidin-4-yl]-1-oxazol-2-yl-butan-1-one;
4-(1-[1,8]Naphthyridin-2-ylmethyl-piperidin-4-yl)-1-oxazol-2-yl-butan-1-one;
1-Oxazol-2-yl-4-(1-quinoxalin-2-ylmethyl-piperidin-4-yl)-butan-1-one;
4-(1-Furan-2-ylmethyl-piperidin-4-yl)-1-oxazol-2-yl-butan-1-one;
4-[4-Oxo-4-(5-pyridin-2-yl-oxazol-2-yl)-butyl]-piperidine-1-carboxylic acid tert-butyl ester;
4-[4-(5-Furan-2-yl-oxazol-2-yl)-4-oxo-butyl]-piperidine-1-carboxylic acid tert-butyl ester;
4-(1-Benzyl-piperidin-4-yl)-1-(5-furan-2-yl-oxazol-2-yl)-butan-1-one;
1-Oxazol-2-yl-4-[1-(2,4,6-trifluoro-benzyl)-piperidin-4-yl]-butan-1-one;
1-Oxazol-2-yl-4-[1-(2,3,5-trifluoro-benzyl)-piperidin-4-yl]-butan-1-one;
4-[1-(2,2-Difluoro-benzo[1,3]dioxol-5-ylmethyl)-piperidin-4-yl]-1-oxazol-2-yl-butan-1-one;
4-(1-Heptyl-piperidin-4-yl)-1-oxazol-2-yl-butan-1-one;
4-(1-Nonyl-piperidin-4-yl)-1-oxazol-2-yl-butan-1-one;
4-[1-(3-Methyl-butyl)-piperidin-4-yl]-1-oxazol-2-yl-butan-1-one; and
1-Oxazol-2-yl-4-(1-pentyl-piperidin-4-yl)-butan-1-one;
and pharmaceutically acceptable salts thereof.

29. A pharmaceutical composition according to claim 27, further comprising: an analgesic selected from the group consisting of opioids and non-steroidal anti-inflammatory drugs.

30. A pharmaceutical composition according to claim 27, further comprising: an additional active ingredient selected from the group consisting of aspirin, acetaminophen, opioids, ibuprofen, naproxen, COX-2 inhibitors, gabapentin, pregabalin, and tramadol.

31. A method of treating a subject suffering from or diagnosed with a disease, disorder, or medical condition mediated by FAAH activity, comprising administering to the subject in need of such treatment an effective amount of a compound of Formula (I): 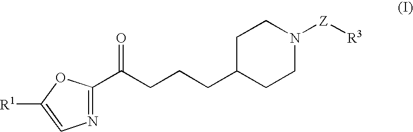
wherein:
R¹ is —H; a —CO₂C₁₋₄alkyl or —CO₂H group; or a phenyl, pyridyl, pyridazinyl, pyrimidinyl, pyrazinyl, thiophenyl, furanyl, or oxazolyl group, each unsubstituted or substituted with —CO₂H or —CO₂C₁₋₄alkyl;
Z is —C(O)—, —CO₂—, —SO₂—, —C(O)NH—, —CH₂—, or —CH(CH₃); and
R³ is:
(a) —(CH₂)ₙ—R⁴, where n is 0, 1, or 2, and R⁴ is:
(i) phenyl, unsubstituted or substituted with one, or two, or three R^{a} moieties or where two adjacent R^{a} moieties together form —O(CH₂)₁₋₂O— or —O(CF₂)O—;
where each R^{a} moiety is —C₁₋₇alkyl, —C₃₋₇cycloalkyl, —C₂₋₇alkenyl, —OH, —OC₁₋₇alkyl, —OC₃₋₇cycloalkyl, phenyl unsubstituted or substituted with R^{b}, phenoxy unsubstituted or substituted with R^{b}, furanyl, thiophenyl, imidazolyl, fluoro, chloro, bromo, iodo, —CF₃, —OCF₃, —SC₁₋₄alkyl, —SO₂C₁₋₄alkyl, —SOC₁₋₄alkyl, —CN, —CO₂C₁₋₄alkyl, —CO₂H, —COC₁₋₄alkyl, —SO₂NR^{c}R^{d}, —NR^{c}SO₂R^{d}, —C(O)NR^{c}R^{d}, —NR^{c}C(O)R^{d}, or —N(R^{c})R^{d};
where R^{b} is selected from the group consisting of —C₁₋₄alkyl, —OC₁₋₄alkyl, fluoro, chloro, bromo, iodo, —CN, —OH, —CF₃, —OCF₃, and —NO₂; and
where R^{c} and R^{d} are each independently —H or —C₁₋₇alkyl, or R^{c} and R^{d} taken together form a 3- to 7-membered heterocycloalkyl ring;
(ii) a five- or six-membered monocyclic heteroaryl ring, unsubstituted or substituted with one or two R^{e} moieties as defined above;
(iii) naphthyl, unsubstituted or substituted one or two R^{e} moieties, where each R^{e} moiety is independently selected from the group consisting of —C₁₋₄alkyl, —OC₁₋₄alkyl, fluoro, chloro, bromo, iodo, —CN, —OH, —CF₃, —OCF₃, and —NO₂;
(iv) a nine- or ten-membered fused bicyclic heteroaryl, unsubstituted or substituted with one or two R^{e} moieties; or
(v) —C₃₋₉cycloalkyl;
(b) —(CH₂)ₓO(CH₂)_{y}R⁴, where when Z is —C(O)—, —SO₂—, or —CH₂—, x is 1 or 2 and y is 0, 1, or 2, and when Z is —CO₂— or —C(O)NH—, x is 2 and y is 0, 1, or 2;
(c) —C₂₋₉alkyl; or
(d) —C₂₋₉alkyl, where one carbon chain member is replaced by nitrogen or oxygen;
or a pharmaceutically acceptable salt, pharmaceutically acceptable prodrug, or pharmaceutically active metabolite of such compound.

32. A method according to claim 31, wherein said compound is selected from the group consisting of:
4-[4-(5-Carboxy-oxazol-2-yl)-4-oxo-butyl]-piperidine-1-carboxylic acid tert-butyl ester;
1-Oxazol-2-yl-4-[1-(3,4,5-trifluoro-benzyl)-piperidin-4-yl]-butan-1-one;
4-[1-(6-Isopropyl-pyridin-3-ylmethyl)-piperidin-4-yl]-1-oxazol-2-yl-butan-1-one;
4-[1-(4-Chloro-3-trifluoromethyl-benzyl)-piperidin-4-yl]-1-oxazol-2-yl-butan-1-one;
4-[1-(4-Cyclohexyloxy-benzyl)-piperidin-4-yl]-1-oxazol-2-yl-butan-1-one;
4-[1-(3-Isopropoxy-benzyl)-piperidin-4-yl]-1-oxazol-2-yl-butan-1-one;
4-[1-(3-Cyclohexyloxy-benzyl)-piperidin-4-yl]-1-oxazol-2-yl-butan-1-one;
4-[1-(3-Fluoro-4-trifluoromethyl-benzyl)-piperidin-4-yl]-1-oxazol-2-yl-butan-1-one;
4-[1-(2-Fluoro-biphenyl-4-ylmethyl)-piperidin-4-yl]-1-oxazol-2-yl-butan-1-one;
4-[1-(6-tert-Butyl-pyridin-3-ylmethyl)-piperidin-4-yl]-1-oxazol-2-yl-butan-1-one;
4-[1-(3-Isopropyl-benzyl)-piperidin-4-yl]-1-oxazol-2-yl-butan-1-one;
4-[1-(4-Imidazol-1-yl-benzyl)-piperidin-4-yl]-1-oxazol-2-yl-butan-1-one;
1-Oxazol-2-yl-4-[1-(1-phenyl-ethyl)-piperidin-4-yl]-butan-1-one;
2-[4-(1-Biphenyl-3-ylmethyl-piperidin-4-yl)-butyryl]-oxazole-5-carboxylic acid;
2-{4-[1-(4-Isopropyl-benzyl)-piperidin-4-yl]-butyryl}-oxazole-5-carboxylic acid;
2-{4-[1-(3-Phenoxy-benzyl)-piperidin-4-yl]-butyryl}-oxazole-5-carboxylic acid;
2-{4-[1-(Toluene-4-sulfonyl)-piperidin-4-yl]-butyryl}-oxazole-5-carboxylic acid;
6-(2-{4-[1-(4-Isopropyl-benzyl)-piperidin-4-yl]-butyryl}-oxazol-5-yl)-pyridine-2-carboxylic acid methyl ester;
6-(2-{4-[1-(4-Isopropyl-benzyl)-piperidin-4-yl]-butyryl}-oxazol-5-yl)-pyridine-2-carboxylic acid;
6-{2-[4-(1-Biphenyl-3-ylmethyl-piperidin-4-yl)-butyryl]-oxazol-5-yl}-pyridine-2-carboxylic acid;
6-(2-{4-[1-(3-Cyclohexyloxy-benzyl)-piperidin-4-yl]-butyryl}-oxazol-5-yl)-pyridine-2-carboxylic acid; and
6-(2-{4-[1-(Toluene-4-sulfonyl)-piperidin-4-yl]-butyryl}-oxazol-5-yl)-pyridine-2-carboxylic acid;
and pharmaceutically acceptable salts thereof.

33. A method according to claim 31, wherein the disease, disorder, or medical condition is selected from the group consisting of: anxiety, pain, sleep disorders, eating disorders, inflammation, movement disorders, HIV wasting syndrome, closed head injury, stroke, Alzheimer's disease, epilepsy, Tourette's syndrome, Niemann-Pick disease, Parkinson's disease, Huntington's chorea, optic neuritis, autoimmune uveitis, drug withdrawal, nausea, emesis, sexual dysfunction, post-traumatic stress disorder, cerebral vasospasm, glaucoma, irritable bowel syndrome, inflammatory bowel disease, immunosuppression, gastroesophageal reflux disease, paralytic ileus, secretory diarrhea, gastric ulcer, rheumatoid arthritis, unwanted pregnancy, hypertension, cancer, hepatitis, allergic airway disease, autoimmune diabetes, intractable pruritis, and neuroinflammation.

34. A method according to claim 31, wherein the disease, disorder, or medical condition is selected from the group consisting of: anxiety, pain, inflammation, sleep disorders, eating disorders, and movement disorders.

35. A method according to claim 31, wherein the disease, disorder, or medical condition is multiple sclerosis.

36. A method according to claim 31, wherein the disease, disorder, or medical condition is pain or inflammation.

37. A method of treating a subject suffering from or diagnosed with a disease, disorder, or medical condition mediated by FAAH activity, comprising administering to the subject in need of such treatment an effective amount of a compound of Formula (IA): 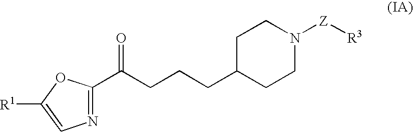
wherein:
R¹ is —H, or a phenyl, pyridyl, pyridazinyl, pyrimidinyl, pyrazinyl, thiophenyl, furanyl, or oxazolyl group;
Z is —C(O)—, —CO₂—, —SO₂—, —C(O)NH—, or —CH₂—; and
R³ is:
(a) —(CH₂)ₙ—R⁴, where n is 0, 1, or 2, and R⁴ is:
(i) phenyl, unsubstituted or substituted with one, or two, or three R^{a} moieties or where two adjacent R^{a} moieties together form —O(CH₂)₁₋₂O— or —O(CF₂)O—;
where each R^{a} moiety is —C₁₋₇alkyl, —C₃₋₇cycloalkyl, —C₂₋₇alkenyl, —OH, —OC₁₋₇alkyl, phenyl unsubstituted or substituted with R^{b}, phenoxy unsubstituted or substituted with R^{b}, furanyl, thiophenyl, fluoro, chloro, bromo, iodo, —CF₃, —OCF₃, —SC₁₋₄alkyl, —SO₂C₁₋₄alkyl, —SOC₁₋₄-alkyl, —CN, —CO₂C₁₋₄alkyl, —CO₂H, —COC₁₋₄alkyl, —SO₂NR^{c}R^{d}, —NR^{c}SO₂R^{d}, —C(O)NR^{c}R^{d}, —NR^{c}C(O)R^{d}, or —N(R^{c})R^{d};
where R^{b} is selected from the group consisting of —C₁₋₄alkyl, —OC₁₋₄alkyl, fluoro, chloro, bromo, iodo, —CN, —OH, —CF₃, —OCF₃, and —NO₂; and
where R^{c} and R^{d} are each independently —H or —C₁₋₇alkyl, or R^{c} and R^{d} taken together form a 3- to 7-membered heterocycloalkyl ring;
(ii) a five- or six-membered monocyclic heteroaryl ring, unsubstituted or substituted with one or two R^{a} moieties as defined above;
(iii) naphthyl, unsubstituted or substituted one or two R^{e} moieties, where each R^{e} moiety is independently selected from the group consisting of —C₁₋₄alkyl, —OC₁₋₄alkyl, fluoro, chloro, bromo, iodo, —CN, —OH, —CF₃, —OCF₃, and —NO₂;
(iv) a nine- or ten-membered fused bicyclic heteroaryl, unsubstituted or substituted with one or two R^{e} moieties; or
(v) —C₃₋₉cycloalkyl;
(b) —(CH₂)ₓO(CH₂)_{y}R⁴, where when Z is —C(O)—, —SO₂—, or —CH₂—, x is 1 or 2 and y is 0, 1, or 2, and when Z is —CO₂— or —C(O)NH—, x is 2 and y is 0, 1, or 2;
(c) —C₂₋₉alkyl; or
(d) —C₂₋₉alkyl, where one carbon chain member is replaced by nitrogen or oxygen;
or a pharmaceutically acceptable salt, pharmaceutically acceptable prodrug, or pharmaceutically active metabolite of such compound.

38. A method according to claim 37, wherein said compound is selected from the group consisting of:
4-(4-Oxazol-2-yl-4-oxo-butyl)-piperidine-1-carboxylic acid tert-butyl ester;
1-Oxazol-2-yl-4-[1-(3-phenoxy-benzyl)-piperidin-4-yl]-butan-1-one;
4-(1-Benzyl-piperidin-4-yl)-1-oxazol-2-yl-butan-1-one;
1-Oxazol-2-yl-4-(1-pyridin-2-ylmethyl-piperidin-4-yl)-butan-1-one;
1-Oxazol-2-yl-4-(1-pyridin-3-ylmethyl-piperidin-4-yl)-butan-1-one;
1-Oxazol-2-yl-4-(1-pyridin-4-ylmethyl-piperidin-4-yl)-butan-1-one;
4-[1-(4-Fluoro-benzyl)-piperidin-4-yl]-1-oxazol-2-yl-butan-1-one;
4-[1-(3-Fluoro-benzyl)-piperidin-4-yl]-1-oxazol-2-yl-butan-1-one;
4-[1-(4-Chloro-benzyl)-piperidin-4-yl]-1-oxazol-2-yl-butan-1-one;
4-[1-(3-Chloro-benzyl)-piperidin-4-yl]-1-oxazol-2-yl-butan-1-one;
4-[1-(3,4-Dibromo-benzyl)-piperidin-4-yl]-1-oxazol-2-yl-butan-1-one;
4-[1-(3,4-Dichloro-benzyl)-piperidin-4-yl]-1-oxazol-2-yl-butan-1-one;
4-[1-(3-Chloro-4-fluoro-benzyl)-piperidin-4-yl]-1-oxazol-2-yl-butan-1-one;
4-(1-Benzo[1,3]dioxol-5-ylmethyl-piperidin-4-yl)-1-oxazol-2-yl-butan-1-one;
1-Oxazol-2-yl-4-[1-(4-phenoxy-benzyl)-piperidin-4-yl]-butan-1-one;
4-[1-(4-Methoxy-benzyl)-piperidin-4-yl]-1-oxazol-2-yl-butan-1-one;
4-[1-(3-Methoxy-benzyl)-piperidin-4-yl]-1-oxazol-2-yl-butan-1-one;
4-[1-(4-Methyl-benzyl)-piperidin-4-yl]-1-oxazol-2-yl-butan-1-one;
4-[1-(3-Methyl-benzyl)-piperidin-4-yl]-1-oxazol-2-yl-butan-1-one;
4-(1-Naphthalen-2-ylmethyl-piperidin-4-yl)-1-oxazol-2-yl-butan-1-one;
1-Oxazol-2-yl-4-(1-quinolin-3-ylmethyl-piperidin-4-yl)-butan-1-one;
4-[1-(4-Isopropyl-benzyl)-piperidin-4-yl]-1-oxazol-2-yl-butan-1-one;
4-[1-(4-Isopropoxy-benzyl)-piperidin-4-yl]-1-oxazol-2-yl-butan-1-one;
4-[1-(4-tert-Butoxy-benzyl)-piperidin-4-yl]-1-oxazol-2-yl-butan-1-one;
3,3-Dimethyl-1-[4-(4-oxazol-2-yl-4-oxo-butyl)-piperidin-1-yl]-butan-1-one;
3-Methyl-1-[4-(4-oxazol-2-yl-4-oxo-butyl)-piperidin-1-yl]-butan-1-one;
1-Oxazol-2-yl-4-(1-phenylacetyl-piperidin-4-yl)-butan-1-one;
4-(1-Benzoyl-piperidin-4-yl)-1-oxazol-2-yl-butan-1-one;
4-(1-Cyclohexanecarbonyl-piperidin-4-yl)-1-oxazol-2-yl-butan-1-one;
4-(1-Isobutyryl-piperidin-4-yl)-1-oxazol-2-yl-butan-1-one;
4-(1-Cyclopentanecarbonyl-piperidin-4-yl)-1-oxazol-2-yl-butan-1-one;
4-[1-(3-Cyclopentyl-propionyl)-piperidin-4-yl]-1-oxazol-2-yl-butan-1-one;
1-Oxazol-2-yl-4-[1-(2-phenoxy-acetyl)-piperidin-4-yl]-butan-1-one;
4-[1-(2-Benzyloxy-acetyl)-piperidin-4-yl]-1-oxazol-2-yl-butan-1-one;
4-{1-[2-(4-Chloro-phenoxy)-acetyl]-piperidin-4-yl}-1-oxazol-2-yl-butan-1-one;
1-Oxazol-2-yl-4-[1-(3-phenyl-propionyl)-piperidin-4-yl]-butan-1-one;
1-Oxazol-2-yl-4-{1-[3-(4-phenoxy-phenyl)-propionyl]-piperidin-4-yl}-butan-1-one;
1-Oxazol-2-yl-4-[1-(2-p-tolyl-acetyl)-piperidin-4-yl]-butan-1-one;
1-Oxazol-2-yl-4-[1-(2-m-tolyl-acetyl)-piperidin-4-yl]-butan-1-one;
4-{1-[2-(4-Chloro-phenyl)-acetyl]-piperidin-4-yl}-1-oxazol-2-yl-butan-1-one;
4-{1-[2-(3-Chloro-phenyl)-acetyl]-piperidin-4-yl}-1-oxazol-2-yl-butan-1-one;
4-{1-[2-(2-Chloro-phenyl)-acetyl]-piperidin-4-yl}-1-oxazol-2-yl-butan-1-one;
4-{1-[2-(3-Methyl-isoxazol-5-yl)-acetyl]-piperidin-4-yl}-1-oxazol-2-yl-butan-1-one;
1-Oxazol-2-yl-4-[1-(3-p-tolyl-propionyl)-piperidin-4-yl]-butan-1-one;
1-Oxazol-2-yl-4-[1-(3-o-tolyl-propionyl)-piperidin-4-yl]-butan-1-one;
4-{1-[3-(4-Chloro-phenyl)-propionyl]-piperidin-4-yl}-1-oxazol-2-yl-butan-1-one;
1-Oxazol-2-yl-4-[1-(3-pyridin-3-yl-propionyl)-piperidin-4-yl]-butan-1-one;
4-[1-(2-Cyclopentyl-acetyl)-piperidin-4-yl]-1-oxazol-2-yl-butan-1-one;
4-[1-(2-Cyclohexyl-acetyl)-piperidin-4-yl]-1-oxazol-2-yl-butan-1-one;
4-[1-(3-Cyclohexyl-propionyl)-piperidin-4-yl]-1-oxazol-2-yl-butan-1-one;
4-Methyl-1-[4-(4-oxazol-2-yl-4-oxo-butyl)-piperidin-1-yl]-pentan-1-one;
1-Oxazol-2-yl-4-[1-(toluene-4-sulfonyl)-piperidin-4-yl]-butan-1-one;
1-Oxazol-2-yl-4-(1-phenylmethanesulfonyl-piperidin-4-yl)-butan-1-one;
1-Oxazol-2-yl-4-[1-(2-phenyl-ethanesulfonyl)-piperidin-4-yl]-butan-1-one;
4-[1-(4-Fluoro-benzenesulfonyl)-piperidin-4-yl]-1-oxazol-2-yl-butan-1-one;
1-Oxazol-2-yl-4-[1-(propane-2-sulfonyl)-piperidin-4-yl]-butan-1-one;
1-Oxazol-2-yl-4-[1-(propane-1-sulfonyl)-piperidin-4-yl]-butan-1-one;
4-[1-(Butane-1-sulfonyl)-piperidin-4-yl]-1-oxazol-2-yl-butan-1-one;
4-(1-Benzenesulfonyl-piperidin-4-yl)-1-oxazol-2-yl-butan-1-one;
4-[1-(4-Chloro-benzenesulfonyl)-piperidin-4-yl]-1-oxazol-2-yl-butan-1-one;
4-[1-(4-Methoxy-benzenesulfonyl)-piperidin-4-yl]-1-oxazol-2-yl-butan-1-one;
4-[1-(3,4-Dichloro-benzenesulfonyl)-piperidin-4-yl]-1-oxazol-2-yl-butan-1-one;
4-(4-Oxazol-2-yl-4-oxo-butyl)-piperidine-1-carboxylic acid benzyl ester;
4-(4-Oxazol-2-yl-4-oxo-butyl)-piperidine-1-carboxylic acid ethyl ester;
4-(4-Oxazol-2-yl-4-oxo-butyl)-piperidine-1-carboxylic acid 2-methoxy-ethyl ester;
4-(4-Oxazol-2-yl-4-oxo-butyl)-piperidine-1-carboxylic acid 2-benzyloxy-ethyl ester;
4-(4-Oxazol-2-yl-4-oxo-butyl)-piperidine-1-carboxylic acid 2,2-dimethyl-propyl ester;
4-(4-Oxazol-2-yl-4-oxo-butyl)-piperidine-1-carboxylic acid isobutyl ester;
4-(4-Oxazol-2-yl-4-oxo-butyl)-piperidine-1-carboxylic acid isopropyl ester;
4-(4-Oxazol-2-yl-4-oxo-butyl)-piperidine-1-carboxylic acid propyl ester;
4-[1-(4-Ethyl-benzyl)-piperidin-4-yl]-1-oxazol-2-yl-butan-1-one;
4-(1-Biphenyl-3-ylmethyl-piperidin-4-yl)-1-oxazol-2-yl-butan-1-one;
4-(1-Biphenyl-4-ylmethyl-piperidin-4-yl)-1-oxazol-2-yl-butan-1-one;
4-[1-(6-Methoxy-pyridin-3-ylmethyl)-piperidin-4-yl]-1-oxazol-2-yl-butan-1-one;
4-[1-(6-Chloro-pyridin-3-ylmethyl)-piperidin-4-yl]-1-oxazol-2-yl-butan-1-one;
4-[1-(6-Bromo-pyridin-3-ylmethyl)-piperidin-4-yl]-1-oxazol-2-yl-butan-1-one;
4-[1-(6-Bromo-pyridin-2-ylmethyl)-piperidin-4-yl]-1-oxazol-2-yl-butan-1-one;
4-[1-(5-Bromo-pyridin-3-ylmethyl)-piperidin-4-yl]-1-oxazol-2-yl-butan-1-one;
4-[1-(6-Methyl-pyridin-2-ylmethyl)-piperidin-4-yl]-1-oxazol-2-yl-butan-1-one;
4-[1-(2-Methyl-benzyl)-piperidin-4-yl]-1-oxazol-2-yl-butan-1-one;
4-[1-(2,3-Difluoro-benzyl)-piperidin-4-yl]-1-oxazol-2-yl-butan-1-one;
4-[1-(4-Isobutyl-benzyl)-piperidin-4-yl]-1-oxazol-2-yl-butan-1-one;
4-[1-(4-tert-Butyl-benzyl)-piperidin-4-yl]-1-oxazol-2-yl-butan-1-one;
4-[1-(2-Chloro-benzyl)-piperidin-4-yl]-1-oxazol-2-yl-butan-1-one;
4-[1-(2-Bromo-benzyl)-piperidin-4-yl]-1-oxazol-2-yl-butan-1-one;
4-(1-Cyclohexylmethyl-piperidin-4-yl)-1-oxazol-2-yl-butan-1-one;
4-[1-(2-Methoxy-benzyl)-piperidin-4-yl]-1-oxazol-2-yl-butan-1-one;
4-[1-(4-Dimethylamino-benzyl)-piperidin-4-yl]-1-oxazol-2-yl-butan-1-one;
4-[1-(4-Diethylamino-benzyl)-piperidin-4-yl]-1-oxazol-2-yl-butan-1-one;
4-[1-(3-Bromo-benzyl)-piperidin-4-yl]-1-oxazol-2-yl-butan-1-one;
4-[1-(4-Bromo-benzyl)-piperidin-4-yl]-1-oxazol-2-yl-butan-1-one;
1-Oxazol-2-yl-4-(1-quinolin-2-ylmethyl-piperidin-4-yl)-butan-1-one;
1-Oxazol-2-yl-4-(1-quinolin-4-ylmethyl-piperidin-4-yl)-butan-1-one;
4-[1-(2,3-Dimethyl-benzyl)-piperidin-4-yl]-1-oxazol-2-yl-butan-1-one;
1-Oxazol-2-yl-4-(1-phenethyl-piperidin-4-yl)-butan-1-one;
1-Oxazol-2-yl-4-[1-(6-p-tolyloxy-pyridin-3-ylmethyl)-piperidin-4-yl]-butan-1-one;
4-[1-(2-Chloro-quinolin-3-ylmethyl)-piperidin-4-yl-]-1-oxazol-2-yl-butan-1-one;
4-[1-(2-Chloro-6-methyl-quinolin-3-ylmethyl)-piperidin-4-yl]-1-oxazol-2-yl-butan-1-one;
4-[1-(2-Chloro-8-methyl-quinolin-3-ylmethyl)-piperidin-4-yl]-1-oxazol-2-yl-butan-1-one;
4-[1-(2-Chloro-6-methoxy-quinolin-3-ylmethyl)-piperidin-4-yl]-1-oxazol-2-yl-butan-1-one;
4-[1-(4-Cyclohexyl-benzyl)-piperidin-4-yl]-1-oxazol-2-yl-butan-1-one;
1-Oxazol-2-yl-4-[1-(4-pyrrolidin-1-yl-benzyl)-piperidin-4-yl]-butan-1-one;
1-Oxazol-2-yl-4-[1-(4-piperidin-1-yl-benzyl)-piperidin-4-yl]-butan-1-one;
4-{1-[6-(3-Methoxy-phenyl)-pyridin-3-ylmethyl]-piperidin-4-yl}-1-oxazol-2-yl-butan-1-one;
1-Oxazol-2-yl-4-[1-(6-phenoxy-pyridin-3-ylmethyl)-piperidin-4-yl]-butan-1-one;
4-[1-(4-Morpholin-4-yl-benzyl)-piperidin-4-yl]-1-oxazol-2-yl-butan-1-one;
4-[1-(6-Morpholin-4-yl-pyridin-3-ylmethyl)-piperidin-4-yl]-1-oxazol-2-yl-butan-1-one;
1-Oxazol-2-yl-4-[1-(3,4,5,6-tetrahydro-2H-[1,2]bipyridinyl-5′-ylmethyl)-piperidin-4-yl]-butan-1-one;
4-[1-(6-Furan-2-yl-pyridin-3-ylmethyl)-piperidin-4-yl]-1-oxazol-2-yl-butan-1-one;
1-Oxazol-2-yl-4-[1-(6-thiophen-2-yl-pyridin-3-ylmethyl)-piperidin-4-yl]-butan-1-one;
1-Oxazol-2-yl-4-[1-(6-thiophen-3-yl-pyridin-3-ylmethyl)-piperidin-4-yl]-butan-1-one;
3-{5-[4-(4-Oxazol-2-yl-4-oxo-butyl)-piperidin-1-ylmethyl]-pyridin-2-yl}-benzonitrile;
4-[1-(2,5-Difluoro-benzyl)-piperidin-4-yl]-1-oxazol-2-yl-butan-1-one;
4-[1-(2,4-Difluoro-benzyl)-piperidin-4-yl]-1-oxazol-2-yl-butan-1-one;
4-[1-(3,4-Difluoro-benzyl)-piperidin-4-yl]-1-oxazol-2-yl-butan-1-one;
4-(1-[1,8]Naphthyridin-2-ylmethyl-piperidin-4-yl)-1-oxazol-2-yl-butan-1-one;
1-Oxazol-2-yl-4-(1-quinoxalin-2-ylmethyl-piperidin-4-yl)-butan-1-one;
4-(1-Furan-2-ylmethyl-piperidin-4-yl)-1-oxazol-2-yl-butan-1-one;
4-[4-Oxo-4-(5-pyridin-2-yl-oxazol-2-yl)-butyl]-piperidine-1-carboxylic acid tert-butyl ester;
4-[4-(5-Furan-2-yl-oxazol-2-yl)-4-oxo-butyl]-piperidine-1-carboxylic acid tert-butyl ester;
4-(1-Benzyl-piperidin-4-yl)-1-(5-furan-2-yl-oxazol-2-yl)-butan-1-one;
1-Oxazol-2-yl-4-[1-(2,4,6-trifluoro-benzyl)-piperidin-4-yl]-butan-1-one;
1-Oxazol-2-yl-4-[1-(2,3,5-trifluoro-benzyl)-piperidin-4-yl]-butan-1-one;
4-[1-(2,2-Difluoro-benzo[1,3]dioxol-5-ylmethyl)-piperidin-4-yl]-1-oxazol-2-yl-butan-1-one;
4-(1-Heptyl-piperidin-4-yl)-1-oxazol-2-yl-butan-1-one;
4-(1-Nonyl-piperidin-4-yl)-1-oxazol-2-yl-butan-1-one;
4-[1-(3-Methyl-butyl)-piperidin-4-yl]-1-oxazol-2-yl-butan-1-one; and
1-Oxazol-2-yl-4-(1-pentyl-piperidin-4-yl)-butan-1-one;
and pharmaceutically acceptable salts thereof.

39. A method according to claim 37, wherein the disease, disorder, or medical condition is selected from the group consisting of: anxiety, pain, sleep disorders, eating disorders, inflammation, movement disorders, HIV wasting syndrome, closed head injury, stroke, Alzheimer's disease, epilepsy, Tourette's syndrome, Niemann-Pick disease, Parkinson's disease, Huntington's chorea, optic neuritis, autoimmune uveitis, drug withdrawal, nausea, emesis, sexual dysfunction, post-traumatic stress disorder, cerebral vasospasm, glaucoma, irritable bowel syndrome, inflammatory bowel disease, immunosuppression, gastroesophageal reflux disease, paralytic ileus, secretory diarrhea, gastric ulcer, rheumatoid arthritis, unwanted pregnancy, hypertension, cancer, hepatitis, allergic airway disease, autoimmune diabetes, intractable pruritis, and neuroinflammation.

40. A method according to claim 37, wherein the disease, disorder, or medical condition is selected from the group consisting of: anxiety, pain, inflammation, sleep disorders, eating disorders, and movement disorders.

41. A method according to claim 37, wherein the disease, disorder, or medical condition is multiple sclerosis.

42. A method according to claim 37, wherein the disease, disorder, or medical condition is pain or inflammation.

43. A process of making a compound of Formula (IA): 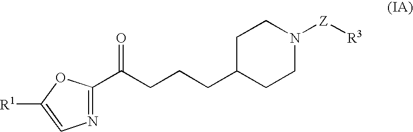
or a pharmaceutically acceptable salt, pharmaceutically acceptable prodrug, or pharmaceutically active metabolite of such compound;
comprising:
(a) reacting an oxazole (IV): 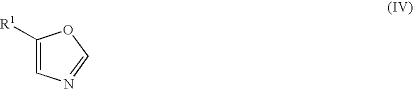
with iPrMgHal in an organic solvent to form an organic mixture; and
(b) treating the organic mixture with 4-[3-(methoxy-methyl-carbamoyl)-propyl]-piperidine-1-carboxylic acid tert-butyl ester to form a compound of formula (V): 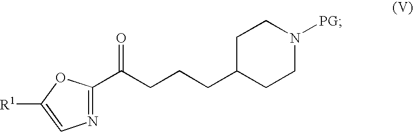
wherein:
R¹ is —H, or a phenyl, pyridyl, pyridazinyl, pyrimidinyl, pyrazinyl, thiophenyl, furanyl, or oxazolyl group;
Z is —C(O)—, —CO₂—, —SO₂—, —C(O)NH—, or —CH₂—; and
R³ is:
(a) —(CH₂)ₙ—R⁴, where n is 0, 1, or 2, and R⁴ is:
(i) phenyl, unsubstituted or substituted with one, or two, or three R^{a} moieties or where two adjacent R^{a} moieties together form —O(CH₂)₁₋₂O— or —O(CF₂)O—;
where each R^{a} moiety is —C₁₋₇alkyl, —C₃₋₇cycloalkyl, —C₂₋₇alkenyl, —OH, —OC₁₋₇alkyl, phenyl unsubstituted or substituted with R^{b}, phenoxy unsubstituted or substituted with R^{b}, furanyl, thiophenyl, fluoro, chloro, bromo, iodo, —CF₃, —OCF₃, —SC₁₋₄alkyl, —SO₂C₁₋₄alkyl, —SOC₁₋₄alkyl, —CN, —CO₂C₁₋₄alkyl, —CO₂H, —COC₁₋₄alkyl, —SO₂NR^{c}R^{d}, —NR^{c}SO₂R^{d}, —C(O)NR^{c}R^{d}, —NR^{c}C(O)R^{d}, or —N(R^{c})R^{d};
where R^{b} is selected from the group consisting of —C₁₋₄alkyl, —OC₁₋₄alkyl, fluoro, chloro, bromo, iodo, —CN, —OH, —CF₃, —OCF₃, and —NO₂; and
where R^{c} and R^{d} are each independently —H or —C₁₋₇alkyl, or R^{c} and R^{d} taken together form a 3- to 7-membered heterocycloalkyl ring;
(ii) a five- or six-membered monocyclic heteroaryl ring, unsubstituted or substituted with one or two R^{a} moieties as defined above;
(iii) naphthyl, unsubstituted or substituted one or two R^{e} moieties, where each R^{e} moiety is independently selected from the group consisting of —C₁₋₄alkyl, —OC₁₋₄alkyl, fluoro, chloro, bromo, iodo, —CN, —OH, —CF₃, —OCF₃, and —NO₂;
(iv) a nine- or ten-membered fused bicyclic heteroaryl, unsubstituted or substituted with one or two R^{e} moieties; or
(v) —C₃₋₉cycloalkyl;
(b) —(CH₂)ₓO(CH₂)_{y}R⁴, where when Z is —C(O)—, —SO₂—, or —CH₂—, x is 1 or 2 and y is 0, 1, or 2, and when Z is —CO₂— or —C(O)NH—, x is 2 and y is 0, 1, or 2;
(c) —C₂₋₉alkyl; or
(d) —C₂₋₉alkyl, where one carbon chain member is replaced by nitrogen or oxygen;
Hal is Cl or Br; and
PG is tert-butoxycarbonyl.

44. A process as defined in claim 43, wherein the reacting of the oxazole (IV) with iPrMgHal is performed at a temperature of from about −30° C. to about 0° C.

45. A process as defined in claim 43, wherein the reacting of the oxazole (IV) with iPrMgHal is performed at a temperature of from about −15° C. to about 0° C.

46. A process as defined in claim 43, wherein the treating of the organic mixture with 4-[3-(methoxy-methyl-carbamoyl)-propyl]-piperidine-1-carboxylic acid tert-butyl ester is performed at a temperature of from about 0° C. to the reflux temperature of the solvent.

47. A process as defined in claim 43, wherein the treating of the organic mixture with 4-[3-(methoxy-methyl-carbamoyl)-propyl]-piperidine-1-carboxylic acid tert-butyl ester is performed at a temperature of from about 0° C. to about 25° C.

48. A process as defined in claim 43, wherein R¹ is —H, pyridyl, or furanyl.

49. A process as defined in claim 43, wherein Hal is Cl.

50. A process as defined in claim 43, wherein the reacting with iPrMgHal is performed using two molar equivalents of iPrMgHal relative to one molar equivalent of oxazole (IV).

51. A process as defined in claim 43, wherein the organic solvent is tetrahydrofuran.

52. A process as defined in claim 43, further comprising treating a compound of formula (V) with TFA to give an amine of formula (VI): 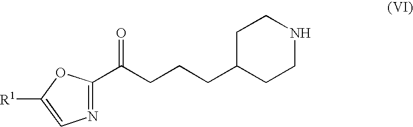
wherein R¹ is as previously defined.
